SEQUENCE LISTING (12 sequences)

SEQ ID NO: 1 - DNA, 55 bp, synthetic construct
  variation 29..36: note = n is a, c, g, or t
  source 1..55: mol_type = other DNA
acacgacgctcttccgatctagcatcacnnnnnnnnttttttttttttttttttv

SEQ ID NO: 2 - DNA, 55 bp, synthetic construct
  variation 29..36: note = n is a, c, g, or t
  source 1..55: mol_type = other DNA
acacgacgctcttccgatctcatatacannnnnnnnttttttttttttttttttv

SEQ ID NO: 3 - DNA, 55 bp, synthetic construct
  variation 29..36: note = n is a, c, g, or t
  source 1..55: mol_type = other DNA
acacgacgctcttccgatctgtaatgttnnnnnnnnttttttttttttttttttv

SEQ ID NO: 4 - DNA, 55 bp, synthetic construct
  variation 29..36: note = n is a, c, g, or t
  source 1..55: mol_type = other DNA
acacgacgctcttccgatcttgattagtnnnnnnnnttttttttttttttttttv

SEQ ID NO: 5 - DNA, 49 bp, synthetic construct
  variation 29..36: note = n is a, c, g, or t
  source 1..49: mol_type = other DNA
acacgacgctcttccgatctagcatcacnnnnnnnncagacagacttgt

SEQ ID NO: 6 - DNA, 49 bp, synthetic construct
  variation 29..36: note = n is a, c, g, or t
  source 1..49: mol_type = other DNA
acacgacgctcttccgatctcatatacannnnnnnncagacagacttgt

SEQ ID NO: 7 - DNA, 49 bp, synthetic construct
  variation 29..36: note = n is a, c, g, or t
  source 1..49: mol_type = other DNA
acacgacgctcttccgatctgtaatgttnnnnnnnncagacagacttgt

SEQ ID NO: 8 - DNA, 49 bp, synthetic construct
  variation 29..36: note = n is a, c, g, or t
  source 1..49: mol_type = other DNA
acacgacgctcttccgatcttgattagtnnnnnnnncagacagacttgt

SEQ ID NO: 9 - DNA, 49 bp, synthetic construct
  variation 29..36: note = n is a, c, g, or t
  source 1..49: mol_type = other DNA
acacgacgctcttccgatctagcatcacnnnnnnnnccttttgggtgtg

SEQ ID NO: 10 - DNA, 49 bp, synthetic construct
  variation 29..36: note = n is a, c, g, or t
  source 1..49: mol_type = other DNA
acacgacgctcttccgatctcatatacannnnnnnnccttttgggtgtg

SEQ ID NO: 11 - DNA, 49 bp, synthetic construct
  variation 29..36: note = n is a, c, g, or t
  source 1..49: mol_type = other DNA
acacgacgctcttccgatctgtaatgttnnnnnnnnccttttgggtgtg

SEQ ID NO: 12 - DNA, 49 bp, synthetic construct
  variation 29..36: note = n is a, c, g, or t
  source 1..49: mol_type = other DNA
acacgacgctcttccgatcttgattagtnnnnnnnnccttttgggtgtg

(19) United States
(12) Patent Application Publication
(10) Pub. No.: US 2025/0277261 A1
(43) Pub. Date: Sep. 4, 2025

(54) ERROR CORRECTION IN AMPLIFICATION OF SAMPLES

(71) Applicant: Becton, Dickinson and Company, Franklin Lakes, NJ (US)

(72) Inventor: Eleen Shum, San Jose, CA (US)

(21) Appl. No.: 19/209,707

(22) Filed: May 15, 2025

Related U.S. Application Data
(63) Continuation of application No. 18/053,603, filed on Nov. 8, 2022, now Pat. No. 12,331,351.
Application No. 18/053,603 is a continuation of application No. 16/232,287, filed on Dec. 26, 2018, now Pat. No. 11,525,157.
Application No. 16/232,287 is a continuation of application No. 15/608,780, filed on May 30, 2017, now Pat. No. 10,202,641.
(60) Provisional application No. 62/343,689, filed on May 31, 2016.

(51) Int. Cl. C12Q 1/686

(52) U.S. Cl. CPC C12Q 1/686

(57) ABSTRACT

Disclosed herein are methods and systems for correcting errors in sample amplification, including the errors occurred in determining the number of targets in samples. In some embodiments, the method comprises: stochastically barcoding a plurality of targets in the samples using oligonucleotides comprising stochastic barcodes to generate stochastically barcoded targets; contacting one or more defined barcoded primers with each of the one or more samples; and determining an amplification noise.

### RELATED APPLICATION

The present application is a continuation of U.S. patent application Ser. No. 18/053,603, filed on Nov. 8, 2022, which is a continuation of U.S. patent application Ser. No. 16/232,287, filed on Dec. 26, 2018, now U.S. Pat. No. 11,525,157, which is a continuation of U.S. patent application Ser. No. 15/608,780, filed on May 30, 2017, now U.S. Pat. No. 10,202,641, which claims priority under 35 U.S.C. § 119 (e) to U.S. Provisional Application No. 62/343,689, filed on May 31, 2016. The content of these related applications is incorporated herein by reference in its entirety.

### REFERENCE TO SEQUENCE LISTING

The present application is being filed along with a Sequence Listing in electronic format. The Sequence Listing is provided as a file entitled 68EB-298692-US3_SequenceListing, created Jan. 30, 2023, which is 16.1 kilobytes in size. The information in the electronic format of the Sequence Listing is incorporated herein by reference in its entirety.

### BACKGROUND

### Field

The present disclosure relates generally to the field of molecular biology and more particularly error correction in amplification of samples with stochastically barcoded targets.

### Description of the Related Art

Methods and techniques such as stochastic barcoding are useful for cell analysis, in particular deciphering gene expression profiles to determine the states of cells using, for example, reverse transcription, polymerase chain reaction (PCR) amplification, and next generation sequencing (NGS). However, these methods and techniques can introduce errors caused by, for example, PCR crossover that may result in over-estimated molecular counts.

Thus, there is a need for methods and techniques capable of correcting errors caused by, for example, PCR crossover in order to attain accurate molecular counts estimated using stochastic barcoding.

### SUMMARY

Disclosed herein are methods for determining the number of targets in one or more samples. In some embodiments, the method comprises: stochastically barcoding a plurality of targets in each of one or more samples using a plurality of oligonucleotides comprising stochastic barcodes to generate stochastically barcoded targets, wherein for each sample, each stochastic barcode comprises a molecular label and an identical sample label, wherein the molecular labels of at least two stochastic barcodes differ from one another by at least one nucleotide; contacting one or more defined barcoded primers with each of the one or more samples, wherein each of the one or more defined barcoded primers comprises a defined sample label and a defined molecular label, and wherein the defined sample labels are variants of the sample labels; amplifying the stochastically barcoded targets and the one or more defined barcoded primers to generate a plurality of amplified stochastically barcoded targets and a plurality of amplified defined barcoded primers; and estimating the number of each of the plurality of targets, wherein estimating the number of each of the plurality of targets comprises: determining a pre-correction number of each of the plurality of targets using the molecular label; determining an amplification noise by determining the number of defined molecular labels with different sequences in the plurality of amplified defined barcoded primers; and removing the amplification noise from the pre-correction number of each of the plurality of targets to generate the estimated number of each of the plurality of targets. The method can be multiplexed.

In some embodiments, contacting the one or more defined barcoded primers with each of the one or more samples comprises introducing the one or more defined barcoded primers at the same concentration of the plurality of oligonucleotides comprising the stochastic barcodes. The defined molecular label can be 5-20 nucleotides in length. The defined molecular label and the molecular label can have the same length. The molecular label can comprise 5-20 nucleotides. Some of the molecular labels of the stochastic barcodes and some of the defined molecular labels of the one or more defined barcoded primers can have the same sequence. The molecular labels of different stochastic barcodes can be different from one another. The defined sample label can be 5-20 nucleotides in length.

In some embodiments, the one or more defined barcoded primers can comprise one or more types of defined barcoded primers. The defined molecular labels of one type of defined barcoded primers can differ from one another by at least one nucleotide. Some defined molecular labels of different types of defined barcoded primers can have the same sequence. Different types of defined barcoded primers can have the same length. The lengths of different types of defined barcoded primers can differ by at most 10 nucleotides. The defined sample labels of different types of defined barcoded primers can have different sequences. The sample label and the defined sample label can have the same length.

In some embodiments, the Hamming distance between the sample labels of the stochastic barcodes and the defined sample labels of the defined barcoded primers can be at least 2 or 4. The stochastic barcodes and the one or more types of defined barcoded primers can have the same length or different lengths.

In some embodiments, the method further comprise: removing at least 50% of the oligonucleotides comprising stochastic barcodes not incorporated into the stochastically barcoded targets and the one or more defined barcoded primers from each of the one or more samples. Less than 10% of the unincorporated oligonucleotides comprising the stochastic barcodes may not be removed from each of the one or more samples. The percentage of the one or more defined barcoded primers not removed from each of the one or more samples can be substantially the same as the percentage of the plurality of oligonucleotides comprising the stochastic barcodes not removed from each of the one or more samples. The percentage of the one or more defined barcoded primers not removed from each of the one or more samples can be within 10% of the percentage of the plurality of oligonucleotides comprising the stochastic barcodes not removed from each of the one or more samples.

In some embodiments, amplifying the stochastically barcoded targets and the one or more defined barcoded primers to generate the plurality of amplified stochastically barcoded targets and the plurality of amplified defined barcoded primers comprises amplifying the stochastically barcoded targets and the one or more defined barcoded primers by polymerase chain reaction (PCR).

In some embodiments, determining the pre-correction number of each of the plurality of targets using the molecular label comprises: determining sequences of molecular labels of the amplified stochastically barcoded targets; and counting the number of the molecular labels with different sequences. Determining the sequences of the molecular labels of the amplified stochastically barcoded targets can comprise sequencing some or all of the plurality of amplified stochastically barcoded targets. Sequencing some or all of the amplified stochastically barcoded targets can comprise generating sequences each with a read length of 100 or more bases.

In some embodiments, determining the number of defined molecular labels with different sequences in the plurality of amplified defined barcoded primers comprises: determining sequences of the defined molecular labels of the amplified defined barcoded primers; and counting the number of defined molecular labels with different sequences. Determining the sequences of the defined molecular labels of the amplified defined barcoded primers can comprise sequencing some or all of the plurality of amplified defined barcoded primers. Sequencing some or all of the plurality of amplified defined barcoded primers can comprise generating sequences each with a read length of 100 or more bases.

In some embodiments, determining the amplification noise comprises determining the number of defined molecular labels with different sequences in the plurality of amplified stochastically barcoded targets comprises determining the number of defined molecular labels in the plurality of amplified defined barcoded primers with different sequences for each type of amplified defined barcoded primers. Determining the amplification noise comprises determining the number of defined molecular labels with different sequences in the plurality of amplified stochastically barcoded can comprise determining the average number of defined molecular labels in the plurality of amplified defined barcoded primers with different sequences for different types of amplified defined barcoded primers.

In some embodiments, determining the amplification noise comprises determining the number of defined molecular labels with different sequences in the plurality of amplified stochastically barcoded targets and the plurality of amplified defined barcoded primers can comprise determining the maximum number of defined molecular labels in the plurality of amplified stochastically barcoded targets and the plurality of amplified defined barcoded primers with different sequences for different types of amplified defined barcoded primers. The amplification noise can comprise noise caused by PCR crossover. In some embodiments, the method can comprise pooling the stochastically barcoded targets to generate a pool of stochastically barcoded targets.

In some embodiments, stochastically barcoding the plurality of targets comprises hybridizing the plurality of oligonucleotides comprising the stochastic barcodes with the plurality of targets to generate the stochastically barcoded targets.

In some embodiments, stochastically barcoding the plurality of targets can be performed with a solid support comprising the plurality of oligonucleotides comprising the stochastic barcodes. The solid support can comprise a plurality of synthetic particles associated with the plurality of oligonucleotides comprising the stochastic barcodes. The synthetic particles can be beads. The beads can be silica gel beads, controlled pore glass beads, magnetic beads, Dynabeads, Sephadex/Sepharose beads, cellulose beads, polystyrene beads, or any combination thereof. The solid support can comprise a polymer, a matrix, a hydrogel, a needle array device, an antibody, or any combination thereof.

In some embodiments, the one or more samples comprise cells. At least one of the one or more samples can comprise a single cell. In some embodiments, the method can comprise lysing the cells. Lysing the cells can comprise heating the one or more samples, contacting the one or more samples with a detergent, changing the pH of the one or more samples, or any combination thereof. The cells can comprise one or more cell types. At least one of the one or more cell types can be brain cell, heart cell, cancer cell, circulating tumor cell, organ cell, epithelial cell, metastatic cell, benign cell, primary cell, circulatory cell, or any combination thereof. The plurality of targets can comprise ribonucleic acids (RNAs), messenger RNAs (mRNAs), microRNAs, small interfering RNAs (siRNAs), RNA degradation products, RNAs each comprising a poly(A) tail, and any combination thereof.

Disclosed herein are methods for determining amplification noise. In some embodiments, the method comprise: reverse transcribing a plurality targets in each of one or more samples using oligonucleotides comprising reverse transcription primers to generate reverse transcribed targets; contacting one or more defined barcoded primers with each of the one or more samples, wherein each of the one or more defined barcoded primers comprises a defined molecular label; amplifying the reverse transcribed targets and the one or more defined barcoded primers to generate a plurality of amplified reverse transcribed targets and a plurality of amplified defined barcoded primers; and determining an amplification noise by determining the number of defined molecular labels with different sequences in the plurality of defined barcoded primers. The method can be multiplexed.

In some embodiments, for each sample, each oligonucleotide comprises a stochastic barcode, wherein the stochastic barcode comprises a molecular label and an identical sample label, wherein the molecular labels of at least two plurality of stochastic barcodes differ from one another by at least one nucleotide, wherein each of the one or more defined barcoded primers comprises a defined sample label, wherein the defined sample labels are variants of the sample label, and wherein the Hamming distance between the sample labels of stochastic barcodes and the defined sample labels of the defined barcoded primers is at least 2.

In some embodiments, contacting the one or more defined barcoded primers with each of the one or more samples comprises introducing the one or more defined barcoded primers at the same concentration of the oligonucleotides comprising the reverse transcription primers. The defined molecular label can be 5-20 nucleotides in length. The defined molecular label and the molecular label can have the same length. Some of the molecular labels of the stochastic barcodes and some of the defined molecular labels of the one or more defined barcoded primers can have the same sequence. The molecular label can comprise 5-20 nucleotides. The molecular labels of different reverse stochastic barcodes can be different from one another. The defined sample label can be 5-20 nucleotides in length.

In some embodiments, the one or more defined barcoded primers comprise one or more types of defined barcoded primers. The defined molecular labels of one type of defined barcoded primers differ from one another by at least one nucleotide. Some defined molecular labels of different types of defined barcoded primers can have the same sequence. Different types of defined barcoded primers can have the same length. The lengths of different types of defined barcoded primer can differ by at most 10 nucleotides. The sample labels of different types of defined barcoded primer can have different sequences. The sample label and the defined sample label can have the same length.

In some embodiments, the Hamming distance between the sample labels of the stochastic barcodes and the defined sample labels of the defined barcoded primers can be at least 4. The stochastic barcodes and the one or more types of defined barcoded primers have the same length. The stochastic barcodes and the one or more types of defined barcoded primers can have different lengths.

In some embodiments, the method comprises removing at least 50% of the oligonucleotides comprising the reverse transcription primers not incorporated into the reverse transcribed targets and the one or more defined barcoded primers each of the one or more samples. Less than 10% of the unincorporated oligonucleotides comprising the reverse transcription primers may not be removed from each of the one or more samples. The percentage of the one or more defined barcoded primers not removed from each of the one or more samples can be substantially the same as the percentage of the oligonucleotides comprising the reverse transcription primers not removed from each of the one or more samples. The percentage of the one or more defined barcoded primers not removed from each of the one or more samples can be within 10% of the percentage of the oligonucleotides comprising the reverse transcription primers not removed from each of the one or more samples.

In some embodiments, amplifying the reverse transcribed targets and the one or more defined barcoded primers to generate the plurality of amplified reverse transcribed targets and the plurality of amplified defined barcoded primers comprises amplifying the reverse transcribed targets and the one or more defined barcoded primers by polymerase chain reaction (PCR).

In some embodiments, determining the number of defined molecular labels with different sequences in the plurality of amplified defined barcoded primers comprises: determining sequences of defined molecular labels of the amplified defined barcoded primers; and counting the number of defined molecular labels with different sequences for each type amplified defined barcoded primers. Determining the sequences of the defined molecular labels of the amplified defined barcoded primers can comprise sequencing some or all of the amplified defined barcoded primers in the plurality of amplified defined barcoded primers. Sequencing some or all of the amplified defined barcoded primers can comprise generating sequences each with a read length of 100 or more bases.

In some embodiments, determining the number of defined molecular labels with different sequences in the plurality of amplified reverse transcribed targets comprises determining the number of defined molecular labels in the plurality of amplified reverse transcribed targets with different sequences for each type of amplified defined barcoded primers. Determining the number of defined molecular labels with different sequences in the plurality of amplified defined barcoded primers can comprise determining the average number of the molecular labels with different sequences for different types of amplified defined barcoded primers.

In some embodiments, determining the number of defined molecular labels with different sequences in the plurality of amplified defined barcoded primers comprises determining the maximum number of the molecular labels with different sequences for different types of amplified defined barcoded primers. The amplification noise can comprise noise caused by PCR crossover. In some embodiments, the method comprises pooling the reverse transcribed targets to generate a pool of reverse transcribed targets.

In some embodiments, the method comprises estimating the number of each of the plurality of targets. Estimating the number of each of the plurality of targets can comprise: determining sequences of molecular labels of the amplified reverse transcribed targets; and counting the number of the molecular labels with different sequences. Estimating the number of each of the plurality of targets can comprise: determining a pre-correction number of each of the targets using the molecular label; and removing the amplification noise from the pre-correction number of each of the plurality of targets to generate the estimated number of each of the targets.

In some embodiments, reverse transcribing the plurality of targets using oligonucleotides comprising reverse transcription primers comprises hybridizing the oligonucleotides comprising the reverse transcription primers with the plurality of targets to generate the reverse transcribed targets.

In some embodiments reverse transcribing the plurality of targets using the reverse transcription primers can be performed with a solid support comprising the oligonucleotides comprising the reverse transcription primers. The solid support can comprise a plurality of synthetic particles associated with the oligonucleotides comprising the reverse transcription primers. The synthetic particles can be beads. The beads can be silica gel beads, controlled pore glass beads, magnetic beads, Dynabeads, Sephadex/Sepharose beads, cellulose beads, polystyrene beads, or any combination thereof. The solid support can comprise a polymer, a matrix, a hydrogel, a needle array device, an antibody, or any combination thereof.

In some embodiments, the one or more samples comprise cells. At least one of the one or more samples can comprise a single cell. In some embodiments, the method comprises lysing the cells. Lysing the cells can comprise heating the one or more samples, contacting the one or more samples with a detergent, changing the pH of the one or more samples, or any combination thereof. The cells can comprise one or more cell types. At least one of the one or more cell types can be brain cell, heart cell, cancer cell, circulating tumor cell, organ cell, epithelial cell, metastatic cell, benign cell, primary cell, circulatory cell, or any combination thereof. The plurality of targets can comprise ribonucleic acids (RNAs), messenger RNAs (mRNAs), microRNAs, small interfering RNAs (siRNAs), RNA degradation products, RNAs each comprising a poly(A) tail, and any combination thereof.

Disclosed herein are methods for determining the number of targets in microwells of a microwell array. In some embodiments, the method comprises: stochastically barcoding a plurality of targets in each of one or more samples in microwells of a microwell array using a plurality of oligonucleotides comprising stochastic barcodes to generate stochastically barcoded targets, wherein for each well, each stochastic barcode comprises a molecular label and an identical sample label, wherein the molecular labels of at least two stochastic barcodes differ from one another by at least one nucleotide; pooling the stochastically barcoded targets from the microwells of the microwell array to generate a pool of stochastically barcoded targets; contacting defined barcoded primers of one or more types of defined barcoded primers with each of the one or more samples, wherein each defined barcoded primer comprises a defined sample label and a defined molecular label, wherein the defined sample labels of defined barcoded primers of the same type of defined barcoded primers have the same sequence, wherein the defined sample labels of different types of defined barcoded primers have different sequences, wherein the sample labels of the stochastic barcodes and the defined sample labels are variants of the sample label, and wherein the Hamming distance between the sample labels of the stochastic barcodes and the defined sample labels of the defined barcoded primers is at least 2; amplifying the stochastically barcoded targets and the one or more defined barcoded primers to generate a plurality of amplified stochastically barcoded targets and a plurality of amplified defined barcoded primers; and estimating the number of each of the plurality of targets, wherein estimating the number of each of the plurality of targets comprises: determining a pre-correction number of each of the plurality of targets using the molecular label; determining an amplification noise by determining the number of defined molecular labels with different sequences in the plurality of amplified defined barcoded primers; and removing the amplification noise from the pre-correction number of each of the plurality of targets to generate the estimated number of each of the plurality of targets.

In some embodiments, contacting the defined barcoded primers with each of the one or more samples can comprise introducing the defined barcoded primers at the same concentration of the plurality of oligonucleotides comprising the stochastic barcodes. The defined molecular label can be 5-20 nucleotides in length. The defined molecular label and the molecular label can have the same length. The molecular labels of some of the stochastic barcodes and the defined molecular labels of some of the defined barcoded primers can have the same sequence. The molecular label can comprise 5-20 nucleotides. The defined sample label can be 5-20 nucleotides in length. The defined molecular labels of some defined barcoded primers of different types of defined barcoded primers can have the same sequence. The defined barcoded primers of different types of defined barcoded primers can have the same length. The lengths of different types of defined barcoded primers can differ by at most 10 nucleotides. The sample label and the defined sample label can have the same length.

In some embodiments, the Hamming distance between the sample labels of the stochastic barcodes and the defined sample labels of the defined barcoded primers can be at least 4. The stochastic barcodes and the defined barcoded primers can have the same length. The stochastic barcodes and some of the defined barcoded primers can have different lengths.

In some embodiments, the method comprises removing at least 50% of the oligonucleotides comprising stochastic barcodes not incorporated into the stochastically barcoded targets and the one or more types of defined barcoded primers from each of the one or more samples. Less than 10% of the unincorporated oligonucleotides comprising the stochastic barcodes may not be removed from each of the one or more samples. The percentage of the one or more defined barcoded primers not removed from each of the one or more samples can be substantially the same as the percentage of the plurality of oligonucleotides comprising the stochastic barcodes not removed from each of the one or more samples. The percentage of the one or more defined barcoded primers not removed from each of the one or more samples can be within 10% of the percentage of the plurality of oligonucleotides comprising the stochastic barcodes not removed from each of the one or more samples.

In some embodiments, amplifying the stochastically barcoded targets and the one or more types of defined barcoded primers to generate the plurality of amplified stochastically barcoded targets and the one or more defined barcoded primers comprises amplifying the stochastically barcoded targets and the one or more types defined barcoded primers by polymerase chain reaction (PCR). Determining the pre-correction number of each of the plurality of targets using the molecular label can comprise: determining sequences of molecular labels of the amplified stochastically barcoded targets; and counting the number of the molecular labels with different sequences. Determining the sequences of the molecular labels of the amplified stochastically barcoded targets can comprise sequencing some or all of the plurality of amplified stochastically barcoded targets. Sequencing some or all of the amplified stochastically barcoded targets can comprise generating sequences each with a read length of 100 or more base.

In some embodiments, determining the number of defined molecular labels with different sequences in the plurality of amplified defined barcoded primers comprises: determining sequences of the defined molecular labels of the amplified defined barcoded primers; and counting the number of defined molecular labels with different sequences. Determining the sequences of the defined molecular labels of the amplified defined barcoded primers can comprise sequencing some or all of the plurality of amplified defined barcoded primers. Sequencing some or all of the plurality of amplified defined barcoded primers can comprise generating sequences each with a read length of 100 or more bases.

In some embodiments, estimating the amplification noise can comprise determining the number of defined molecular labels with different sequences in the plurality of amplified defined barcoded primers comprises determining the number of defined molecular labels in the plurality of amplified defined barcoded primers with different sequences for each type of amplified defined barcoded primers. Estimating the amplification noise can comprise determining the number of defined molecular labels with different sequences in the amplified defined barcoded primers comprises determining the average number of defined molecular labels in the plurality of amplified defined barcoded primers with different sequences for different types of amplified defined barcoded primers. Estimating the amplification noise comprises determining the number of defined molecular labels with different sequences in the plurality of amplified defined barcoded primers can comprise determining the maximum number of defined molecular labels in the plurality of amplified defined barcoded primers with different sequences for different types of amplified defined barcoded primers. In some embodiments, the amplification noise comprises noise caused by PCR crossover. In some embodiments, the method comprises pooling the stochastically barcoded targets to generate a pool of stochastically barcoded targets.

In some embodiments, stochastically barcoding the plurality of targets comprises hybridizing the plurality of oligonucleotides comprising the stochastic barcodes with the plurality of targets to generate the stochastically barcoded targets. Stochastically barcoding the plurality of targets in each of one or more samples in the microwells of the microwell array can be performed with a solid support comprising the plurality of oligonucleotides comprising the stochastic barcodes. The solid support can comprise a plurality of synthetic particles associated with the oligonucleotides comprising the reverse transcription primers. The synthetic particles can be beads. The beads can be silica gel beads, controlled pore glass beads, magnetic beads, Dynabeads, Sephadex/Sepharose beads, cellulose beads, polystyrene beads, or any combination thereof. The solid support can comprise a polymer, a matrix, a hydrogel, a needle array device, an antibody, or any combination thereof.

In some embodiments, the one or more samples comprise cells. At least one of the one or more samples can comprise a single cell. In some embodiments, the method comprises lysing the cells. Lysing the cells can comprise heating the one or more samples, contacting the one or more samples with a detergent, changing the pH of the one or more samples, or any combination thereof. The cells can comprise one or more cell types. At least one of the one or more cell types can be brain cell, heart cell, cancer cell, circulating tumor cell, organ cell, epithelial cell, metastatic cell, benign cell, primary cell, circulatory cell, or any combination thereof. The plurality of targets can comprise ribonucleic acids (RNAs), messenger RNAs (mRNAs), microRNAs, small interfering RNAs (siRNAs), RNA degradation products, RNAs each comprising a poly(A) tail, and any combination thereof.

Disclosed herein are kits for determining the number of targets in one or more samples. In some embodiments, a kit comprises: a plurality of oligonucleotides comprising stochastic barcodes, wherein each stochastic barcode comprises a sample label and a molecular label, wherein the sample labels of at least two stochastic barcodes have different sequences, and wherein the molecular labels of stochastic barcodes with sample labels of the same sequence differ from one another by at least one nucleotide; defined barcoded primers, wherein each defined barcoded primer comprises a defined sample label and a defined molecular label, wherein the defined sample labels of defined barcoded primers are variants of the sample labels of the stochastic barcodes, and wherein the Hamming distance between the sample label and the defined sample label is at least 2; and instructions for using the plurality oligonucleotides comprising the plurality of oligonucleotides and the defined barcoded primers.

In some embodiments, the defined sample label can be 5-20 nucleotides in length and the defined molecular label can be 5-20 nucleotides in length. The defined molecular label and the molecular label have the same length. In some embodiments, different types of defined barcoded primers have the same length. The lengths of different types of defined barcoded primers can differ by at most 10 nucleotides. The sample label and the defined sample label can have the same length.

In some embodiments, the Hamming distance between the sample label and the defined sample label can be at least 2 or 4. The stochastic barcodes and the defined barcoded primers can have the same length. The stochastic barcodes and the defined barcoded primers can have different lengths.

In some embodiments, the plurality of oligonucleotides comprising the stochastic barcodes can be associated with a solid support. The solid support can comprise a plurality of synthetic particles associated with the plurality of oligonucleotides comprising stochastic barcodes. The plurality of synthetic particles can be beads. The beads can be silica gel beads, controlled pore glass beads, magnetic beads, Dynabeads, Sephadex/Sepharose beads, cellulose beads, polystyrene beads, or any combination thereof. The synthetic particles can be magnetic beads. The solid support can comprise a polymer, a matrix, a hydrogel, a needle array device, an antibody, or any combination thereof.

In some embodiments, the kit comprises a buffer. The kit can comprise a cartridge. The solid support can be pre-loaded on a substrate. The kit can comprise one or more reagents for a reverse transcription reaction. The kit can comprise one or more reagents for an amplification reaction.

### BRIEF DESCRIPTION OF THE DRAWINGS

FIG. 1 illustrates a non-limiting exemplary stochastic barcode.

FIG. 2 shows a non-limiting exemplary workflow of stochastic barcoding and digital counting.

FIG. 3 is a schematic illustration showing a non-limiting exemplary process for generating an indexed library of the stochastically barcoded targets from a plurality of targets.

FIG. 4 is a flowchart showing a non-limiting exemplary embodiment of removing amplification noises caused by PCR crossover.

FIGS. 5A-5C depict a non-limiting exemplary cartridge for use in the methods of the disclosure.

FIGS. 6A-6D show bar charts of molecular label counts vs. wells for GAPDH and CD3D before and after correction for PCR amplification noises.

FIG. 7 panels (a)-(d) are non-limiting exemplary plots of molecular label counts vs. the number of reads per molecular label (ML), showing that the amplification noises determined using defined barcoded primers (DBPs) were similar to the noise levels determined using no-template controls (NTCs).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Quantifying small numbers of nucleic acids, for example messenger ribonucleic acid (mRNA) molecules, is clinically important for determining, for example, the genes that are expressed in a cell at different stages of development or under different environmental conditions. However, it can also be very challenging to determine the absolute number of nucleic acid molecules (e.g., mRNA molecules), especially when the number of molecules is very small. Stochastic barcoding with, for example, the Precise™ assay (Cellular Research, Inc. (Palo Alto, CA)) can be used to determine the number of molecules, for example mRNA molecules, in a cell.

However, the number of molecules determined by stochastic barcoding can include PCR amplification noises. PCR amplification noises can be caused by stochastic barcodes still present during PCR amplification after barcode removal with Ampure beads. To reduce PCR amplification noises, rigorous barcode removal can be performed prior to PCR amplification. However, such rigorous barcode removal may also wash away many of the stochastically barcoded molecules giving rise to sequencing “signal.” The methods and systems disclosed herein can preserve the sequencing “signal” while eliminating PCR amplification noises to provide more accurate determinations of, for example, gene expression profiles.

Methods and systems for determining the number of targets in one or more samples are disclosed herein. In some embodiments, the method comprises: stochastically barcoding a plurality of targets in each of one or more samples using a plurality of oligonucleotides comprising stochastic barcodes to generate stochastically barcoded targets, wherein for each sample, each stochastic barcode comprises a molecular label and an identical sample label, wherein the molecular labels of at least two stochastic barcodes differ from one another by at least one nucleotide; contacting one or more defined barcoded primers with each of the one or more samples.

Defined barcoded primers can be variants of the stochastic barcodes. For example, a defined barcoded primer can have a corresponding stochastic barcode, where the defined barcoded primer is a variant of the corresponding stochastic barcode. The defined barcoded primer can have a different sequence in one or more regions as compared to the corresponding stochastic barcode. For example, a defined barcoded primer can comprise a defined sample label and a defined molecular label.

In some embodiments, the defined sample label of a defined barcoded primer can be a variant of the sample label of a stochastic barcode (e.g., the sample label of the corresponding stochastic barcode). For example, the defined sample label differs from the sample label in one, two, three, four, five, six, seven, eight, nine, ten, or a range between any two of these values, nucleotides. The defined sample label can be, for example, 5, 6, 7, 8, 9, 10, 11, 12, 13, 14, 15, 16, 17, 18 19, 20, or a range between any two of these values, nucleotides in length. The sample label can be 5, 6, 7, 8, 9, 10, 11, 12, 13, 14, 15, 16, 17, 18 19, 20, or a range between any two of these values, nucleotides in length. The defined sample label and the sample label, for example, a defined sample label of a defined barcoded primer and a sample label of the corresponding stochastic barcode, can have the same length or different length. For example, the defined sample label and the sample label can both be 8 nucleotides in length. In some embodiments, the sequence of the defined sample label and the sequence of the sample label can differ by one nucleotide. In some embodiments, the Hamming distance between the defined sample label and the sample label can be 2 or 4.

In some embodiments, the defined molecular label of a defined barcoded primer can be a variant of the molecular label of a stochastic barcode (e.g., the molecular label of the corresponding stochastic barcode). For example, the sequence of the defined molecular label and the sequence of the molecular label differ by one, two, three, four, five, six, seven, eight, nine, ten, or a range between any two of these values, nucleotides. The defined molecular label can be, for example, 5, 6, 7, 8, 9, 10, 11, 12, 13, 14, 15, 16, 17, 18 19, 20, or a range between any two of these values, nucleotides in length. The molecular label can be, for example, 5, 6, 7, 8, 9, 10, 11, 12, 13, 14, 15, 16, 17, 18 19, 20, or a range between any two of these values, nucleotides in length. The defined molecular label and the molecular label, for example a defined molecular label of a defined barcoded primer and a molecular label of the corresponding stochastic barcode, can have the same length. For example, the defined molecular label and the molecular label can both be 8 nucleotides in length. The defined molecular labels of different defined barcoded primers can be different from one another. The molecular labels of different stochastic barcodes can be different from one another. Some molecular labels of the stochastic barcodes and some defined molecular labels of the defined barcoded primers can have the same sequence.

In some embodiments, the method comprises amplifying the stochastically barcoded targets and the one or more defined barcoded primers to generate a plurality of amplified stochastically barcoded targets and a plurality of amplified defined barcoded primers; and estimating the number of each of the plurality of targets, wherein estimating the number of each of the plurality of targets comprises: determining a pre-correction number of each of the plurality of targets using the molecular label; determining an amplification noise by determining the number of defined molecular labels with different sequences in the plurality of amplified defined barcoded primers; and removing the amplification noise from the pre-correction number of each of the plurality of targets to generate the estimated number of each of the plurality of targets. The methods can be multiplexed.

Kits for determining the number of targets in one or more samples are also disclosed herein. In some embodiments, the kit comprises: a plurality of oligonucleotides comprising stochastic barcodes, wherein each stochastic barcode comprises a sample label and a molecular label, wherein the sample labels of at least two stochastic barcodes have different sequences, and wherein the molecular labels of stochastic barcodes with sample labels of the same sequence differ from one another by at least one nucleotide; defined barcoded primers, wherein each defined barcoded primer comprises a defined sample label and a defined molecular label, wherein the defined sample labels of defined barcoded primers are variants of the sample labels of the stochastic barcodes, and wherein the Hamming distance between the sample label and the defined sample label is at least 2; and instructions for using the plurality oligonucleotides comprising the plurality of oligonucleotides and the defined barcoded primers.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Springs Harbor Press (Cold Springs Harbor, NY 1989). For purposes of the present disclosure, the following terms are defined below.

As used herein, the term “adaptor” can mean a sequence to facilitate amplification or sequencing of associated nucleic acids. The associated nucleic acids can comprise target nucleic acids. The associated nucleic acids can comprise one or more of spatial labels, target labels, sample labels, indexing label, barcodes, stochastic barcodes, or molecular labels. The adapters can be linear. The adaptors can be pre-adenylated adapters. The adaptors can be double- or single-stranded. One or more adaptor can be located on 5′ or 3′ end of a nucleic acid. When the adaptors comprise known sequences on 5′ and 3′ ends, the known sequences can be the same or different sequences. An adaptor located on the 5′ and/or 3′ ends of a polynucleotide can be capable of hybridizing to one or more oligonucleotides immobilized on a surface. An adapter can, in some embodiments, comprise a universal sequence. A universal sequence can be a region of nucleotide sequence that is common to two or more nucleic acid molecules. The two or more nucleic acid molecules can also have regions of different sequence. Thus, for example, 5′ adapters can comprise identical and/or universal nucleic acid sequences and 3′ adapters can comprise identical and/or universal sequences. A universal sequence that may be present in different members of a plurality of nucleic acid molecules can allow the replication or amplification of multiple different sequences using a single universal primer that is complementary to the universal sequence. Similarly, at least one, two (e.g., a pair) or more universal sequences that may be present in different members of a collection of nucleic acid molecules can allow the replication or amplification of multiple different sequences using at least one, two (e.g., a pair) or more single universal primers that are complementary to the universal sequences. Thus, a universal primer includes a sequence that can hybridize to such a universal sequence. The target nucleic acid sequence-bearing molecules may be modified to attach universal adapters (e.g., non-target nucleic acid sequences) to one or both ends of the different target nucleic acid sequences. The one or more universal primers attached to the target nucleic acid can provide sites for hybridization of universal primers. The one or more universal primers attached to the target nucleic acid can be the same or different from each other.

As used herein the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some embodiments, two or more associated species are “tethered”, “attached”, or “immobilized” to one another or to a common solid or semisolid surface. An association may refer to covalent or non-covalent means for attaching labels to solid or semi-solid supports such as beads. An association may be a covalent bond between a target and a label.

As used herein, the term “complementary” can refer to the capacity for precise pairing between two nucleotides. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence. As used herein, the terms “complement”, “complementary”, and “reverse complement” can be used interchangeably. It is understood from the disclosure that if a molecule can hybridize to another molecule it may be the complement of the molecule that is hybridizing.

As used herein, the term “digital counting” can refer to a method for estimating a number of target molecules in a sample. Digital counting can include the step of determining a number of unique labels that have been associated with targets in a sample. This stochastic methodology transforms the problem of counting molecules from one of locating and identifying identical molecules to a series of yes/no digital questions regarding detection of a set of predefined labels.

As used herein, the term “label” or “labels” can refer to nucleic acid codes associated with a target within a sample. A label can be, for example, a nucleic acid label. A label can be an entirely or partially amplifiable label. A label can be entirely or partially sequenceable label. A label can be a portion of a native nucleic acid that is identifiable as distinct. A label can be a known sequence. A label can comprise a junction of nucleic acid sequences, for example a junction of a native and non-native sequence. As used herein, the term “label” can be used interchangeably with the terms, “index”, “tag,” or “label-tag.” Labels can convey information. For example, in various embodiments, labels can be used to determine an identity of a sample, a source of a sample, an identity of a cell, and/or a target.

As used herein, the term “non-depleting reservoirs” can refer to a pool of stochastic barcodes made up of many different labels. A non-depleting reservoir can comprise large numbers of different stochastic barcodes such that when the non-depleting reservoir is associated with a pool of targets each target is likely to be associated with a unique stochastic barcode. The uniqueness of each labeled target molecule can be determined by the statistics of random choice, and depends on the number of copies of identical target molecules in the collection compared to the diversity of labels. The size of the resulting set of labeled target molecules can be determined by the stochastic nature of the barcoding process, and analysis of the number of stochastic barcodes detected then allows calculation of the number of target molecules present in the original collection or sample. When the ratio of the number of copies of a target molecule present to the number of unique stochastic barcodes is low, the labeled target molecules are highly unique (i.e. there is a very low probability that more than one target molecule will have been labeled with a given label).

As used herein, the term “nucleic acid” refers to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g. altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. “Nucleic acid”, “polynucleotide, “target polynucleotide”, and “target nucleic acid” can be used interchangeably.

A nucleic acid can comprise one or more modifications (e.g., a base modification, a backbone modification), to provide the nucleic acid with a new or enhanced feature (e.g., improved stability). A nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′ or the 5′ hydroxyl moiety of the sugar. In forming nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within nucleic acids, the phosphate groups can commonly be referred to as forming the internucleoside backbone of the nucleic acid. The linkage or backbone can be a 3′ to 5′ phosphodiester linkage.

A nucleic acid can comprise a modified backbone and/or modified internucleoside linkages. Modified backbones can include those that retain a phosphorus atom in the backbone and those that do not have a phosphorus atom in the backbone. Suitable modified nucleic acid backbones containing a phosphorus atom therein can include, for example, phosphorothioates, chiral phosphorothioates, phosphorodithioates, phosphotriesters, aminoalkyl phosphotriesters, methyl and other alkyl phosphonate such as 3′-alkylene phosphonates, 5′-alkylene phosphonates, chiral phosphonates, phosphinates, phosphoramidates including 3′-amino phosphoramidate and aminoalkyl phosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

A nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

A nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage.

A nucleic acid can comprise linked morpholino units (i.e. morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C, 4′-C-oxymethylene linkage thereby forming a bicyclic sugar moiety. The linkage can be a methylene (—CH2-), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A nucleic acid may also include nucleobase (often referred to simply as “base”) modifications or substitutions. As used herein, “unmodified” or “natural” nucleobases can include the purine bases, (e.g. adenine (A) and guanine (G)), and the pyrimidine bases, (e.g. thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C—C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b) indol-2-one), pyridoindole cytidine (H-pyrido (3′,′:4,5) pyrrolo[2,3-d]pyrimidin-2-one).

As used herein, the term “sample” can refer to a composition comprising targets. Suitable samples for analysis by the disclosed methods, devices, and systems include cells, tissues, organs, or organisms.

As used herein, the term “sampling device” or “device” can refer to a device which may take a section of a sample and/or place the section on a substrate. A sample device can refer to, for example, a fluorescence activated cell sorting (FACS) machine, a cell sorter machine, a biopsy needle, a biopsy device, a tissue sectioning device, a microfluidic device, a blade grid, and/or a microtome.

As used herein, the term “solid support” can refer to discrete solid or semi-solid surfaces to which a plurality of stochastic barcodes may be attached. A solid support may encompass any type of solid, porous, or hollow sphere, ball, bearing, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. A plurality of solid supports spaced in an array may not comprise a substrate. A solid support may be used interchangeably with the term “bead.”

A solid support can refer to a “substrate.” A substrate can be a type of solid support. A substrate can refer to a continuous solid or semi-solid surface on which the methods of the disclosure may be performed. A substrate can refer to an array, a cartridge, a chip, a device, and a slide, for example.

As used here, the term, “spatial label” can refer to a label which can be associated with a position in space.

As used herein, the term “stochastic barcode” can refer to a polynucleotide sequence comprising labels. A stochastic barcode can be a polynucleotide sequence that can be used for stochastic barcoding. Stochastic barcodes can be used to quantify targets within a sample. Stochastic barcodes can be used to control for errors which may occur after a label is associated with a target. For example, a stochastic barcode can be used to assess amplification or sequencing errors. A stochastic barcode associated with a target can be called a stochastic barcode-target or stochastic barcode-tag-target.

As used herein, the term “gene-specific stochastic barcode” can refer to a polynucleotide sequence comprising labels and a target-binding region that is gene-specific. A stochastic barcode can be a polynucleotide sequence that can be used for stochastic barcoding. Stochastic barcodes can be used to quantify targets within a sample. Stochastic barcodes can be used to control for errors which may occur after a label is associated with a target. For example, a stochastic barcode can be used to assess amplification or sequencing errors. A stochastic barcode associated with a target can be called a stochastic barcode-target or stochastic barcode-tag-target.

As used herein, the term “stochastic barcoding” can refer to the random labeling (e.g., barcoding) of nucleic acids. Stochastic barcoding can utilize a recursive Poisson strategy to associate and quantify labels associated with targets. As used herein, the term “stochastic barcoding” can be used interchangeably with “gene-specific stochastic barcoding.”

As used here, the term “target” can refer to a composition which can be associated with a stochastic barcode. Exemplary suitable targets for analysis by the disclosed methods, devices, and systems include oligonucleotides, DNA, RNA, mRNA, microRNA, tRNA, and the like. Targets can be single or double stranded. In some embodiments targets can be proteins. In some embodiments targets are lipids.

As used herein, the term “reverse transcriptases” can refer to a group of enzymes having reverse transcriptase activity (i.e., that catalyze synthesis of DNA from an RNA template). In general, such enzymes include, but are not limited to, retroviral reverse transcriptase, retrotransposon reverse transcriptase, retroplasmid reverse transcriptases, retron reverse transcriptases, bacterial reverse transcriptases, group II intron-derived reverse transcriptase, and mutants, variants or derivatives thereof. Non-retroviral reverse transcriptases include non-LTR retrotransposon reverse transcriptases, retroplasmid reverse transcriptases, retron reverse transciptases, and group II intron reverse transcriptases. Examples of group II intron reverse transcriptases include the Lactococcus lactis LI.LtrB intron reverse transcriptase, the Thermosynechococcus elongates TeI4c intron reverse transcriptase, or the Geobacillus stearothermophilus GsI-IIC intron reverse transcriptase. Other classes of reverse transcriptases can include many classes of non-retroviral reverse transcriptases (i.e., retrons, group II introns, and diversity-generating retroelements among others).

The terms “universal adaptor primer,” “universal primer adaptor” or “universal adaptor sequence” are used interchangeably to refer to a nucleotide sequence that can be used to hybridize stochastic barcodes to generate gene-specific stochastic barcodes. A universal adaptor sequence can, for example, be a known sequence that is universal across all stochastic barcodes used in methods of the disclosure. For example, when multiple targets are being labeled using the methods disclosed herein, each of the target-specific sequences may be linked to the same universal adaptor sequence. In some embodiments, more than one universal adaptor sequences may be used in the methods disclosed herein. For example, when multiple targets are being labeled using the methods disclosed herein, at least two of the target-specific sequences are linked to different universal adaptor sequences. A universal adaptor primer and its complement may be included in two oligonucleotides, one of which comprises a target-specific sequence and the other comprises a stochastic barcode. For example, a universal adaptor sequence may be part of an oligonucleotide comprising a target-specific sequence to generate a nucleotide sequence that is complementary to a target nucleic acid. A second oligonucleotide comprising a stochastic barcode and a complementary sequence of the universal adaptor sequence may hybridize with the nucleotide sequence and generate a target-specific stochastic barcode. In some embodiments, a universal adaptor primer has a sequence that is different from a universal PCR primer used in the methods of this disclosure.

### Stochastic Barcodes

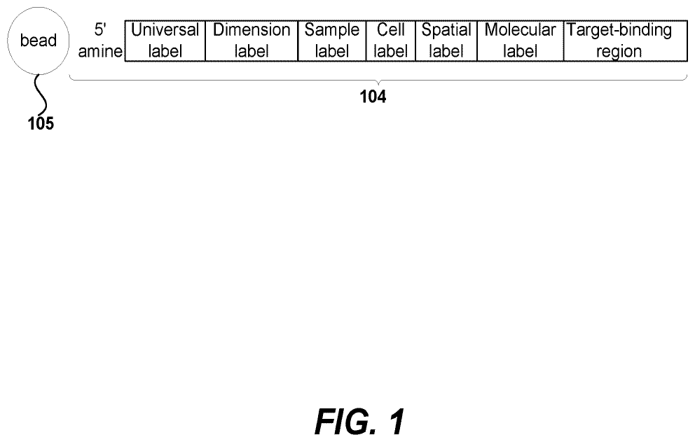

Stochastic barcoding has been described in, for example, US20150299784, WO2015031691, and Fu et al, Proc Natl Acad Sci U.S.A. 2011 May 31;108 (22): 9026-31, the content of these publications is incorporated hereby in its entirety. Briefly, a stochastic barcode can be a polynucleotide sequence that may be used to stochastically label (e.g., barcode, tag) a target. A stochastic barcode can comprise one or more labels. Exemplary labels can include a universal label, a cell label, a molecular label, a sample label, a plate label, a spatial label, and/or a pre-spatial label. FIG. 1 illustrates an exemplary stochastic barcode 104 with a spatial label. The stochastic barcode 104 can comprise a 5′ amine that may link the stochastic barcode to a solid support 105. The stochastic barcode can comprise a universal label, a dimension label, a spatial label, a cell label, and/or a molecular label. The order of different labels (including but not limited to the universal label, the dimension label, the spatial label, the cell label, and the molecule label) in the stochastic barcode can vary. For example, as shown in FIG. 1, the universal label may be the 5′-most label, and the molecular label may be 3′-most label. The spatial label, dimension label, and the cell label may be in any order. In some embodiments, the universal label, the spatial label, the dimension label, the cell label, and the molecular label are in any order.

A label, for example the cell label, can comprise a unique set of nucleic acid sub-sequences of defined length, e.g. seven nucleotides each (equivalent to the number of bits used in some Hamming error correction codes), which can be designed to provide error correction capability. The set of error correction sub-sequences comprise seven nucleotide sequences can be designed such that any pairwise combination of sequences in the set exhibits a defined “genetic distance” (or number of mismatched bases), for example, a set of error correction sub-sequences can be designed to exhibit a genetic distance of three nucleotides. In this case, review of the error correction sequences in the set of sequence data for labeled target nucleic acid molecules (described more fully below) can allow one to detect or correct amplification or sequencing errors. In some embodiments, the length of the nucleic acid sub-sequences used for creating error correction codes can vary, for example, they can be, or be about 1, 2, 3, 4, 5, 6, 7, 8, 9, 10, 15, 20, 30, 31, 40, 50, or a number or a range between any two of these values, nucleotides in length. In some embodiments, nucleic acid sub-sequences of other lengths can be used for creating error correction codes.

The stochastic barcode can comprise a target-binding region. The target-binding region can interact with a target in a sample. The target can be, or comprise, ribonucleic acids (RNAs), messenger RNAs (mRNAs), microRNAs, small interfering RNAs (siRNAs), RNA degradation products, RNAs each comprising a poly(A) tail, or any combination thereof. In some embodiments, the plurality of targets can include deoxyribonucleic acids (DNAs).

In some embodiments, a target-binding region can comprise an oligo(dT) sequence which can interact with poly(A) tails of mRNAs. One or more of the labels of the stochastic barcode (e.g., the universal label, the dimension label, the spatial label, the cell label, and the molecular label) can be separated by a spacer from another one or two of the remaining labels of the stochastic barcode. The spacer can be, for example, 1, 2, 3, 4, 5, 6, 7, 8, 9, 10, 11, 12, 13, 14, 15, 16, 17, 18, 19, or 20 or more nucleotides. In some embodiments, none of the labels of the stochastic barcode is separated by spacer.

### Universal Labels

A stochastic barcode can comprise one or more universal labels. In some embodiments, the one or more universal labels can be the same for all stochastic barcodes in the set of stochastic barcodes attached to a given solid support. In some embodiments, the one or more universal labels can be the same for all stochastic barcodes attached to a plurality of beads. In some embodiments, a universal label can comprise a nucleic acid sequence that is capable of hybridizing to a sequencing primer. Sequencing primers can be used for sequencing stochastic barcodes comprising a universal label. Sequencing primers (e.g., universal sequencing primers) can comprise sequencing primers associated with high-throughput sequencing platforms. In some embodiments, a universal label can comprise a nucleic acid sequence that is capable of hybridizing to a PCR primer. In some embodiments, the universal label can comprise a nucleic acid sequence that is capable of hybridizing to a sequencing primer and a PCR primer. The nucleic acid sequence of the universal label that is capable of hybridizing to a sequencing or PCR primer can be referred to as a primer binding site. A universal label can comprise a sequence that can be used to initiate transcription of the stochastic barcode. A universal label can comprise a sequence that can be used for extension of the stochastic barcode or a region within the stochastic barcode. A universal label can be, or be about, 1, 2, 3, 4, 5, 10, 15, 20, 25, 30, 35, 40, 45, 50, or a number or a range between any two of these values, nucleotides in length. For example, a universal label can comprise at least about 10 nucleotides. A universal label can be at least, or at most, 1, 2, 3, 4, 5, 10, 15, 20, 25, 30, 35, 40, 45, 50, 100, 200, or 300 nucleotides in length. In some embodiments, a cleavable linker or modified nucleotide can be part of the universal label sequence to enable the stochastic barcode to be cleaved off from the support.

### Dimension Labels

A stochastic barcode can comprise one or more dimension labels. In some embodiments, a dimension label can comprise a nucleic acid sequence that provides information about a dimension in which the stochastic labeling occurred. For example, a dimension label can provide information about the time at which a target was stochastically barcoded. A dimension label can be associated with a time of stochastic barcoding in a sample. A dimension label can be activated at the time of stochastic labeling. Different dimension labels can be activated at different times. The dimension label provides information about the order in which targets, groups of targets, and/or samples were stochastically barcoded. For example, a population of cells can be stochastically barcoded at the G0 phase of the cell cycle. The cells can be pulsed again with stochastic barcodes at the G1 phase of the cell cycle. The cells can be pulsed again with stochastic barcodes at the S phase of the cell cycle, and so on. Stochastic barcodes at each pulse (e.g., each phase of the cell cycle), can comprise different dimension labels. In this way, the dimension label provides information about which targets were labelled at which phase of the cell cycle. Dimension labels can interrogate many different biological times. Exemplary biological times can include, but are not limited to, the cell cycle, transcription (e.g., transcription initiation), and transcript degradation. In another example, a sample (e.g., a cell, a population of cells) can be stochastically labeled before and/or after treatment with a drug and/or therapy. The changes in the number of copies of distinct targets can be indicative of the sample's response to the drug and/or therapy.

A dimension label can be activatable. An activatable dimension label can be activated at a specific time point. The activatable label can be, for example, constitutively activated (e.g., not turned off). The activatable dimension label can be, for example, reversibly activated (e.g., the activatable dimension label can be turned on and turned off). The dimension label can be, for example, reversibly activatable at least 1, 2, 3, 4, 5, 6, 7, 8, 9, or 10 or more times. The dimension label can be reversibly activatable, for example, at least 1, 2, 3, 4, 5, 6, 7, 8, 9, or 10 or more times. In some embodiments, the dimension label can be activated with fluorescence, light, a chemical event (e.g., cleavage, ligation of another molecule, addition of modifications (e.g., pegylated, sumoylated, acetylated, methylated, deacetylated, demethylated), a photochemical event (e.g., photocaging), and introduction of a non-natural nucleotide.

The dimension label can, in some embodiments, be identical for all stochastic barcodes attached to a given solid support (e.g., bead), but different for different solid supports (e.g., beads). In some embodiments, at least 60%, 70%, 80%, 85%, 90%, 95%, 97%, 99% or 100% of stochastic barcodes on the same solid support can comprise the same dimension label. In some embodiments, at least 60% of stochastic barcodes on the same solid support can comprise the same dimension label. In some embodiments, at least 95% of stochastic barcodes on the same solid support can comprise the same dimension label.

There can be as many as 106 or more unique dimension label sequences represented in a plurality of solid supports (e.g., beads). A dimension label can be, or be about 1, 2, 3, 4, 5, 10, 15, 20, 25, 30, 35, 40, 45, 50, or a number or a range between any two of these values, nucleotides in length. A dimension label can be at least, or at most, 1, 2, 3, 4, 5, 10, 15, 20, 25, 30, 35, 40, 45, 50, 100, 200, or 300 nucleotides in length. A dimension label can comprise between about 5 to about 200 nucleotides. A dimension label can comprise between about 10 to about 150 nucleotides. A dimension label can comprise between about 20 to about 125 nucleotides in length.

### Spatial Labels

A stochastic barcode can comprise one or more spatial labels. In some embodiments, a spatial label can comprise a nucleic acid sequence that provides information about the spatial orientation of a target molecule which is associated with the stochastic barcode. A spatial label can be associated with a coordinate in a sample. The coordinate can be a fixed coordinate. For example a coordinate can be fixed in reference to a substrate. A spatial label can be in reference to a two or three-dimensional grid. A coordinate can be fixed in reference to a landmark. The landmark can be identifiable in space. A landmark can be a structure which can be imaged. A landmark can be a biological structure, for example an anatomical landmark. A landmark can be a cellular landmark, for instance an organelle. A landmark can be a non-natural landmark such as a structure with an identifiable identifier such as a color code, bar code, magnetic property, fluorescents, radioactivity, or a unique size or shape. A spatial label can be associated with a physical partition (e.g. a well, a container, or a droplet). In some embodiments, multiple spatial labels are used together to encode one or more positions in space.

The spatial label can be identical for all stochastic barcodes attached to a given solid support (e.g., bead), but different for different solid supports (e.g., beads). In some embodiments, the percentage of stochastic barcodes on the same solid support comprising the same spatial label can be, or be about, 60%, 70%, 80%, 85%, 90%, 95%, 97%, 99%, 100%, or a number or a range between any two of these values. In some embodiments, the percentage of stochastic barcodes on the same solid support comprising the same spatial label can be at least, or at most, 60%, 70%, 80%, 85%, 90%, 95%, 97%, 99%, or 100%. In some embodiments, at least 60% of stochastic barcodes on the same solid support can comprise the same spatial label. In some embodiments, at least 95% of stochastic barcodes on the same solid support can comprise the same spatial label.

There can be as many as 106 or more unique spatial label sequences represented in a plurality of solid supports (e.g., beads). A spatial label can be, or be about, 1, 2, 3, 4, 5, 10, 15, 20, 25, 30, 35, 40, 45, 50, or a number or a range between any two of these values, nucleotides in length. A spatial label can be at least or at most 1, 2, 3, 4, 5, 10, 15, 20, 25, 30, 35, 40, 45, 50, 100, 200, or 300 nucleotides in length. A spatial label can comprise between about 5 to about 200 nucleotides. A spatial label can comprise between about 10 to about 150 nucleotides. A spatial label can comprise between about 20 to about 125 nucleotides in length.

### Cell Labels

A stochastic barcodes can comprise one or more cell labels. In some embodiments, a cell label can comprise a nucleic acid sequence that provides information for determining which target nucleic acid originated from which cell. In some embodiments, the cell label is identical for all stochastic barcodes attached to a given solid support (e.g., bead), but different for different solid supports (e.g., beads). In some embodiments, the percentage of stochastic barcodes on the same solid support comprising the same cell label can be, or be about 60%, 70%, 80%, 85%, 90%, 95%, 97%, 99%, 100%, or a number or a range between any two of these values. In some embodiments, the percentage of stochastic barcodes on the same solid support comprising the same cell label can be, or be about 60%, 70%, 80%, 85%, 90%, 95%, 97%, 99%, or 100%. For example, at least 60% of stochastic barcodes on the same solid support can comprise the same cell label. As another example, at least 95% of stochastic barcodes on the same solid support can comprise the same cell label.

There can be as many as 106 or more unique cell label sequences represented in a plurality of solid supports (e.g., beads). A cell label can be, or be about, 1, 2, 3, 4, 5, 10, 15, 20, 25, 30, 35, 40, 45, 50, or a number or a range between any two of these values, nucleotides in length. A cell label can be at least, or at most, 1, 2, 3, 4, 5, 10, 15, 20, 25, 30, 35, 40, 45, 50, 100, 200, or 300 nucleotides in length. For example, a cell label can comprise between about 5 to about 200 nucleotides. As another example, a cell label can comprise between about 10 to about 150 nucleotides. As yet another example, a cell label can comprise between about 20 to about 125 nucleotides in length.

### Molecular Labels

A stochastic barcodes can comprise one or more molecular labels. In some embodiments, a molecular label can comprise a nucleic acid sequence that provides identifying information for the specific type of target nucleic acid species hybridized to the stochastic barcode. A molecular label can comprise a nucleic acid sequence that provides a counter for the specific occurrence of the target nucleic acid species hybridized to the stochastic barcode (e.g., target-binding region).

In some embodiments, a diverse set of molecular labels are attached to a given solid support (e.g., bead). In some embodiments, there can be, or be about, 10², 10³, 10⁴, 10⁵, 10⁶, 10⁷, 10⁸, 10⁹, or a number or a range of unique molecular label sequences. For example, a plurality of stochastic barcodes can comprise about 6561 molecular labels with distinct sequences. As another example, a plurality of stochastic barcodes can comprise about 65536 molecular labels with distinct sequences. In some embodiments, there can be at least, or at most, 10², 10³, 10⁴, 10⁵, 10⁶, 10⁷, 10⁸, or 10⁹, unique molecular label sequences. The unique molecular label sequences attached to a given solid support (e.g., bead).

A molecular label can be, or be about, 1, 2, 3, 4, 5, 10, 15, 20, 25, 30, 35, 40, 45, 50, or a number or a range between any two of these values, nucleotides in length. A molecular label can be at least, or at most, 1, 2, 3, 4, 5, 10, 15, 20, 25, 30, 35, 40, 45, 50, 100, 200, or 300 nucleotides in length.

### Target-Binding Region

A stochastic barcodes can comprise one or more target binding regions. In some embodiments, a target-binding region can hybridize with a target of interest. In some embodiments, the target binding regions can comprise a nucleic acid sequence that hybridizes specifically to a target (e.g. target nucleic acid, target molecule, e.g., a cellular nucleic acid to be analyzed), for example to a specific gene sequence. In some embodiments, a target binding region can comprise a nucleic acid sequence that can attach (e.g., hybridize) to a specific location of a specific target nucleic acid. In some embodiments, the target binding region can comprise a nucleic acid sequence that is capable of specific hybridization to a restriction enzyme site overhang (e.g. an EcoRI sticky-end overhang). The stochastic barcode can then ligate to any nucleic acid molecule comprising a sequence complementary to the restriction site overhang.

In some embodiments, a target binding region can comprise a non-specific target nucleic acid sequence. A non-specific target nucleic acid sequence can refer to a sequence that can bind to multiple target nucleic acids, independent of the specific sequence of the target nucleic acid. For example, target binding region can comprise a random multimer sequence, or an oligo(dT) sequence that hybridizes to the poly(A) tail on mRNA molecules. A random multimer sequence can be, for example, a random dimer, trimer, quatramer, pentamer, hexamer, septamer, octamer, nonamer, decamer, or higher multimer sequence of any length. In some embodiments, the target binding region is the same for all stochastic barcodes attached to a given bead. In some embodiments, the target binding regions for the plurality of stochastic barcodes attached to a given bead can comprise two or more different target binding sequences. A target binding region can be, or be about, 5, 10, 15, 20, 25, 30, 35, 40, 45, 50, or a number or a range between any two of these values, nucleotides in length. A target binding region can be at most about 5, 10, 15, 20, 25, 30, 35, 40, 45, 50 or more nucleotides in length.

In some embodiments, a target-binding region can comprise an oligo(dT) which can hybridize with mRNAs comprising poly-adenylated ends. A target-binding region can be gene-specific. For example, a target-binding region can be configured to hybridize to a specific region of a target. A target-binding region can be, or be about, 1, 2, 3, 4, 5, 6, 7, 8, 9, 10, 11, 12, 13, 14, 15, 16, 17, 18, 19, 20, 21, 22, 23, 24, 25, 26 27, 28, 29, 30, or a number or a range between any two of these values, nucleotides in length. A target-binding region can be at least, or at most, 1, 2, 3, 4, 5, 6, 7, 8, 9, 10, 11, 12, 13, 14, 15, 16, 17, 18, 19, 20, 21, 22, 23, 24, 25, 26 27, 28, 29, or 30, nucleotides in length. A target-binding region can be about 5-30 nucleotides in length. When a stochastic barcode comprises a gene-specific target-binding region, the stochastic barcode can be referred to as a gene-specific stochastic barcode.

### Orientation Property

A stochastic barcode can comprise one or more orientation properties which can be used to orient (e.g., align) the stochastic barcodes. A stochastic barcode can comprise a moiety for isoelectric focusing. Different stochastic barcodes can comprise different isoelectric focusing points. When these stochastic barcodes are introduced to a sample, the sample can undergo isoelectric focusing in order to orient the stochastic barcodes into a known way. In this way, the orientation property can be used to develop a known map of stochastic barcodes in a sample. Exemplary orientation properties can include, electrophoretic mobility (e.g., based on size of the stochastic barcode), isoelectric point, spin, conductivity, and/or self-assembly. For example, stochastic barcodes with an orientation property of self-assembly, can self-assemble into a specific orientation (e.g., nucleic acid nanostructure) upon activation.

### Affinity Property

A stochastic barcode can comprise one or more affinity properties. For example, a spatial label can comprise an affinity property. An affinity property can include a chemical and/or biological moiety that can facilitate binding of the stochastic barcode to another entity (e.g., cell receptor). For example, an affinity property can comprise an antibody, for example, an antibody specific for a specific moiety (e.g., receptor) on a sample. In some embodiments, the antibody can guide the stochastic barcode to a specific cell type or molecule. Targets at and/or near the specific cell type or molecule can be stochastically labeled. The affinity property can, in some embodiments, provide spatial information in addition to the nucleotide sequence of the spatial label because the antibody can guide the stochastic barcode to a specific location. The antibody can be a therapeutic antibody, for example a monoclonal antibody or a polyclonal antibody. The antibody can be humanized or chimeric. The antibody can be a naked antibody or a fusion antibody.

The antibody can be a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment.

The antibody fragment can be, for example, a portion of an antibody such as F(ab′)2, Fab′, Fab, Fv, sFv and the like. In some embodiments, the antibody fragment can bind with the same antigen that is recognized by the full-length antibody. The antibody fragment can include isolated fragments consisting of the variable regions of antibodies, such as the “Fv” fragments consisting of the variable regions of the heavy and light chains and recombinant single chain polypeptide molecules in which light and heavy variable regions are connected by a peptide linker (“scFv proteins”). Exemplary antibodies can include, but are not limited to, antibodies for cancer cells, antibodies for viruses, antibodies that bind to cell surface receptors (CD8, CD34, CD45), and therapeutic antibodies.

### Universal Adaptor Primer

A stochastic barcode can comprise one or more universal adaptor primers. For example, a gene-specific stochastic barcode can comprise a universal adaptor primer. A universal adaptor primer can refer to a nucleotide sequence that is universal across all stochastic barcodes. A universal adaptor primer can be used for building gene-specific stochastic barcodes. A universal adaptor primer can be, or be about, 1, 2, 3, 4, 5, 6, 7, 8, 9, 10, 11, 12, 13, 14, 15, 16, 17, 18, 19, 20, 21, 22, 23, 24, 25, 26 27, 28, 29, 30, or a number or a range between any two of these nucleotides in length. A universal adaptor primer can be at least, or at most, 1, 2, 3, 4, 5, 6, 7, 8, 9, 10, 11, 12, 13, 14, 15, 16, 17, 18, 19, 20, 21, 22, 23, 24, 25, 26 27, 28, 29, or 30 nucleotides in length. A universal adaptor primer can be from 5-30 nucleotides in length.

### Solid Supports

Stochastic barcodes disclosed herein can, in some embodiments, be associated with a solid support. The solid support can be, for example, a synthetic particle. In some embodiments, some or all of the molecular labels (e.g., the first molecular labels) of a plurality of stochastic barcodes (e.g., the first plurality of stochastic barcodes) on a solid support differ by at least one nucleotide. The cell labels of the stochastic barcodes on the same solid support can be the same. The cell labels of the stochastic barcodes on different solid supports can differ by at least one nucleotide. For example, first cell labels of a first plurality of stochastic barcodes on a first solid support can have the same sequence, and second cell labels of a second plurality of stochastic barcodes on a second solid support can have the same sequence. The first cell labels of the first plurality of stochastic barcodes on the first solid support and the second cell labels of the second plurality of stochastic barcodes on the second solid support can differ by at least one nucleotide. A cell label can be, for example, about 5-20 nucleotides long. A molecular label can be, for example, about 5-20 nucleotides long. The synthetic particle can be, for example, a bead.

The bead can be, for example, a silica gel bead, a controlled pore glass bead, a magnetic bead, a Dynabead, a Sephadex/Sepharose bead, a cellulose bead, a polystyrene bead, or any combination thereof. The bead can comprise a material such as polydimethylsiloxane (PDMS), polystyrene, glass, polypropylene, agarose, gelatin, hydrogel, paramagnetic, ceramic, plastic, glass, methylstyrene, acrylic polymer, titanium, latex, sepharose, cellulose, nylon, silicone, or any combination thereof.

In some embodiments, the bead can be a polymeric bead, for example a deformable bead or a gel bead, functionalized with stochastic barcodes (such as gel beads from 10× Genomics (San Francisco, CA). In some implementation, a gel bead can comprise a polymer based gels. Gel beads can be generated, for example, by encapsulating one or more polymeric precursors into droplets. Upon exposure of the polymeric precursors to an accelerator (e.g., tetramethylethylenediamine (TEMED)), a gel bead may be generated.

In some embodiments, the polymeric bead can dissolve, melt, or degrade, for example, under a desired condition. The desired condition can include an environmental condition. The desired condition may result in the polymeric bead dissolving, melting, or degrading in a controlled manner. A gel bead may dissolve, melt, or degrade due to a chemical stimulus, a physical stimulus, a biological stimulus, a thermal stimulus, a magnetic stimulus, an electric stimulus, a light stimulus, or any combination thereof.

Analytes and/or reagents, such as oligonucleotide barcodes, for example, may be coupled/immobilized to the interior surface of a gel bead (e.g., the interior accessible via diffusion of an oligonucleotide barcode and/or materials used to generate an oligonucleotide barcode) and/or the outer surface of a gel bead or any other microcapsule described herein. Coupling/immobilization may be via any form of chemical bonding (e.g., covalent bond, ionic bond) or physical phenomena (e.g., Van der Waals forces, dipole-dipole interactions, etc.). In some cases, coupling/immobilization of a reagent to a gel bead or any other microcapsule described herein may be reversible, such as, for example, via a labile moiety (e.g., via a chemical cross-linker, including chemical cross-linkers described herein). Upon application of a stimulus, the labile moiety may be cleaved and the immobilized reagent set free. In some cases, the labile moiety is a disulfide bond. For example, in the case where an oligonucleotide barcode is immobilized to a gel bead via a disulfide bond, exposure of the disulfide bond to a reducing agent can cleave the disulfide bond and free the oligonucleotide barcode from the bead. The labile moiety may be included as part of a gel bead or microcapsule, as part of a chemical linker that links a reagent or analyte to a gel bead or microcapsule, and/or as part of a reagent or analyte.

In some embodiments, a gel bead can comprise a wide range of different polymers including but not limited to: polymers, heat sensitive polymers, photosensitive polymers, magnetic polymers, pH sensitive polymers, salt-sensitive polymers, chemically sensitive polymers, polyelectrolytes, polysaccharides, peptides, proteins, and/or plastics. Polymers may include but are not limited to materials such as poly(N-isopropylacrylamide) (PNIPAAm), poly(styrene sulfonate) (PSS), poly(allyl amine) (PAAm), poly(acrylic acid) (PAA), poly(ethylene imine) (PEI), poly(diallyldimethyl-ammonium chloride) (PDADMAC), poly(pyrolle) (PPy), poly(vinylpyrrolidone) (PVPON), poly(vinyl pyridine) (PVP), poly(methacrylic acid) (PMAA), poly(methyl methacrylate) (PMMA), polystyrene (PS), poly(tetrahydrofuran) (PTHF), poly(phthaladehyde) (PTHF), poly(hexyl viologen) (PHV), poly(L-lysine) (PLL), poly(L-arginine) (PARG), poly(lactic-co-glycolic acid) (PLGA).

Numerous chemical stimuli can be used to trigger the disruption or degradation of the beads. Examples of these chemical changes may include, but are not limited to pH-mediated changes to the bead wall, disintegration of the bead wall via chemical cleavage of crosslink bonds, triggered depolymerization of the bead wall, and bead wall switching reactions. Bulk changes may also be used to trigger disruption of the beads.

Bulk or physical changes to the microcapsule through various stimuli also offer many advantages in designing capsules to release reagents. Bulk or physical changes occur on a macroscopic scale, in which bead rupture is the result of mechano-physical forces induced by a stimulus. These processes may include, but are not limited to pressure induced rupture, bead wall melting, or changes in the porosity of the bead wall.

Biological stimuli may also be used to trigger disruption or degradation of beads. Generally, biological triggers resemble chemical triggers, but many examples use biomolecules, or molecules commonly found in living systems such as enzymes, peptides, saccharides, fatty acids, nucleic acids and the like. For example, beads may comprise polymers with peptide cross-links that are sensitive to cleavage by specific proteases. More specifically, one example may comprise a microcapsule comprising GFLGK peptide cross links. Upon addition of a biological trigger such as the protease Cathepsin B, the peptide cross links of the shell well are cleaved and the contents of the beads are released. In other cases, the proteases may be heat-activated. In another example, beads comprise a shell wall comprising cellulose. Addition of the hydrolytic enzyme chitosan serves as biologic trigger for cleavage of cellulosic bonds, depolymerization of the shell wall, and release of its inner contents.

The beads may also be induced to release their contents upon the application of a thermal stimulus. A change in temperature can cause a variety changes to the beads. A change in heat may cause melting of a bead such that the bead wall disintegrates. In other cases, the heat may increase the internal pressure of the inner components of the bead such that the bead ruptures or explodes. In still other cases, the heat may transform the bead into a shrunken dehydrated state. The heat may also act upon heat-sensitive polymers within the wall of a bead to cause disruption of the bead.

Inclusion of magnetic nanoparticles to the bead wall of microcapsules may allow triggered rupture of the beads as well as guide the beads in an array. A device of this disclosure may comprise magnetic beads for either purpose. In one example, incorporation of Fe₃O₄ nanoparticles into polyelectrolyte containing beads triggers rupture in the presence of an oscillating magnetic field stimulus.

A bead may also be disrupted or degraded as the result of electrical stimulation. Similar to magnetic particles described in the previous section, electrically sensitive beads can allow for both triggered rupture of the beads as well as other functions such as alignment in an electric field, electrical conductivity or redox reactions. In one example, beads containing electrically sensitive material are aligned in an electric field such that release of inner reagents can be controlled. In other examples, electrical fields may induce redox reactions within the bead wall itself that may increase porosity.

A light stimulus may also be used to disrupt the beads. Numerous light triggers are possible and may include systems that use various molecules such as nanoparticles and chromophores capable of absorbing photons of specific ranges of wavelengths. For example, metal oxide coatings can be used as capsule triggers. UV irradiation of polyelectrolyte capsules coated with SiO₂ may result in disintegration of the bead wall. In yet another example, photo switchable materials such as azobenzene groups may be incorporated in the bead wall. Upon the application of UV or visible light, chemicals such as these undergo a reversible cis-to-trans isomerization upon absorption of photons. In this aspect, incorporation of photon switches result in a bead wall that may disintegrate or become more porous upon the application of a light trigger.

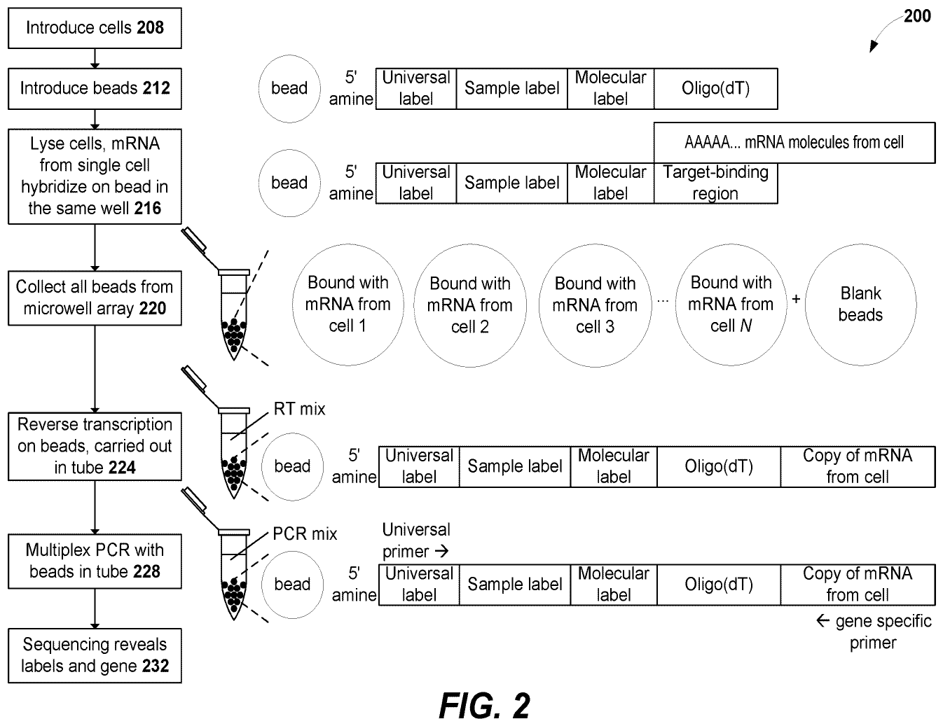

For example, in a non-limiting example of stochastic barcoding illustrated in FIG. 2, after introducing cells such as single cells onto a plurality of microwells of a microwell array at block 208, beads can be introduced onto the plurality of microwells of the microwell array at block 212. Each microwell can comprise one bead. The beads can comprise a plurality of stochastic barcodes. A stochastic barcode can comprise a 5′ amine region attached to a bead. The stochastic barcode can comprise a universal label, a molecular label, a target-binding region, or any combination thereof.

The stochastic barcodes disclosed herein can be associated with (e.g., attached to) a solid support (e.g., a bead). The stochastic barcodes associated with a solid support can each comprise a molecular label selected from a group comprising at least 100 or 1000 molecular labels with unique sequences. In some embodiments, different stochastic barcodes associated with a solid support can comprise molecular labels of different sequences. In some embodiments, a percentage of stochastic barcodes associated with a solid support comprises the same cell label. For example, the percentage can be, or be about 60%, 70%, 80%, 85%, 90%, 95%, 97%, 99%, 100%, or a number or a range between any two of these values. As another example, the percentage can be at least, or at most 60%, 70%, 80%, 85%, 90%, 95%, 97%, 99%, or 100%. In some embodiments, stochastic barcodes associated with a solid support can have the same cell label. The stochastic barcodes associated with different solid supports can have different cell labels selected from a group comprising at least 100 or 1000 cell labels with unique sequences.

The stochastic barcodes disclosed herein can be associated to (e.g., attached to) a solid support (e.g., a bead). In some embodiments, stochastically barcoding the plurality of targets in the sample can be performed with a solid support including a plurality of synthetic particles associated with the plurality of stochastic barcodes. In some embodiments, the solid support can include a plurality of synthetic particles associated with the plurality of stochastic barcodes. The spatial labels of the plurality of stochastic barcodes on different solid supports can differ by at least one nucleotide. The solid support can, for example, include the plurality of stochastic barcodes in two dimensions or three dimensions. The synthetic particles can be beads. The beads can be silica gel beads, controlled pore glass beads, magnetic beads, Dynabeads, Sephadex/Sepharose beads, cellulose beads, polystyrene beads, or any combination thereof. The solid support can include a polymer, a matrix, a hydrogel, a needle array device, an antibody, or any combination thereof. In some embodiments, the solid supports can be free floating. In some embodiments, the solid supports can be embedded in a semi-solid or solid array. The stochastic barcodes may not be associated with solid supports. The stochastic barcodes can be individual nucleotides. The stochastic barcodes can be associated with a substrate.

As used herein, the terms “tethered”, “attached”, and “immobilized” are used interchangeably, and can refer to covalent or non-covalent means for attaching stochastic barcodes to a solid support. Any of a variety of different solid supports can be used as solid supports for attaching pre-synthesized stochastic barcodes or for in situ solid-phase synthesis of stochastic barcode.

In some embodiments, the solid support is a bead. The bead can comprise one or more types of solid, porous, or hollow sphere, ball, bearing, cylinder, or other similar configuration which a nucleic acid can be immobilized (e.g., covalently or non-covalently). The bead can be, for example, composed of plastic, ceramic, metal, polymeric material, or any combination thereof. A bead can be, or comprise, a discrete particle that is spherical (e.g., microspheres) or have a non-spherical or irregular shape, such as cubic, cuboid, pyramidal, cylindrical, conical, oblong, or disc-shaped, and the like. In some embodiments, a bead can be non-spherical in shape.

Beads can comprise a variety of materials including, but not limited to, paramagnetic materials (e.g. magnesium, molybdenum, lithium, and tantalum), superparamagnetic materials (e.g. ferrite (Fe₃O₄; magnetite) nanoparticles), ferromagnetic materials (e.g. iron, nickel, cobalt, some alloys thereof, and some rare earth metal compounds), ceramic, plastic, glass, polystyrene, silica, methylstyrene, acrylic polymers, titanium, latex, sepharose, agarose, hydrogel, polymer, cellulose, nylon, or any combination thereof.

In some embodiments, the bead (e.g., the bead to which the stochastic labels are attached) is a hydrogel bead. In some embodiments, the bead comprises hydrogel.

Some embodiments disclosed herein include one or more particles (for example beads). Each of the particles can comprise a plurality of oligonucleotides (e.g., stochastic barcodes). Each of the plurality of oligonucleotides can comprise a molecular label sequence, a cell label sequence, and a target-binding region (e.g., an oligo dT sequence, a gene-specific sequence, a random multimer, or a combination thereof). The cell label sequence of each of the plurality of oligonucleotides can be the same. The cell label sequences of oligonucleotides on different particles can be different such that the oligonucleotides on different particles can be identified. The number of different cell label sequences can be different in different implementations. In some embodiments, the number of cell label sequences can be, or about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 106, 107, 108, 109, a number or a range between any two of these values, or more. In some embodiments, the number of cell label sequences can be at least, or at most 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 10⁶, 10⁷, 10⁸, or 10⁹. In some embodiments, no more than 1, 2, 3, 4, 5, 6, 7, 8, 9, 10, 20, 30, 40, 50, 60, 70, 80, 90, 100, 200, 300, 400, 500, 600, 700, 800, 900, 1000, or more of the plurality of the particles include oligonucleotides with the same cell sequence. In some embodiment, the plurality of particles that include oligonucleotides with the same cell sequence can be at most 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% or more. In some embodiments, none of the plurality of the particles has the same cell label sequence.

The plurality of oligonucleotides on each particle can comprise different molecular label sequences. In some embodiments, the number of molecular label sequences can be, or about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 10⁶, 10⁷, 10⁸, 10⁹, or a number or a range between any two of these values. In some embodiments, the number of molecular label sequences can be at least, or at most 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 10⁶, 10⁷, 10⁸, or 10⁹. For example, at least 100 of the plurality of oligonucleotides comprise different molecular label sequences. As another example, in a single particle, at least 100, 500, 1000, 5000, 10000, 15000, 20000, 50000, a number or a range between any two of these values, or more of the plurality of oligonucleotides comprise different molecular label sequences. Some embodiments provide a plurality of the particles comprising stochastic barcodes. In some embodiments, the ratio of an occurrence (or a copy or a number) of a target to be labeled and the different molecular label sequences can be at least 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90, or more. In some embodiments, each of the plurality of oligonucleotides further comprises a sample label, a universal label, or both. The particle can be, for example, a nanoparticle or microparticle.

The size of the beads can vary. For example, the diameter of the bead can range from 0.1 micrometer to 50 micrometer. In some embodiments, the diameters of beads can be, or be about, 0.1, 0.5, 1, 2, 3, 4, 5, 6, 7, 8, 9, 10, 20, 30, 40, 50 micrometer, or a number or a range between any two of these values.

The diameters of the bead can be related to the diameter of the wells of the substrate. In some embodiments, the diameters of the bead can be, or be about, 10%, 20%, 30%, 40%, 50%, 60%, 70%, 80%, 90%, 100%, or a number or a range between any two of these values, longer or shorter than the diameter of the well. The diameter of the beads can be related to the diameter of a cell (e.g., a single cell entrapped by a well of the substrate). In some embodiments, the diameters of the beads can be, or be about, 10%, 20%, 30%, 40%, 50%, 60%, 70%, 80%, 90%, 100%, 150%, 200%, 250%, 300%, or a number or a range between any two of these values, longer or shorter than the diameter of the cell.

A bead can be attached to and/or embedded in a substrate. A bead can be attached to and/or embedded in a gel, hydrogel, polymer and/or matrix. The spatial position of a bead within a substrate (e.g., gel, matrix, scaffold, or polymer) can be identified using the spatial label present on the stochastic barcode on the bead which can serve as a location address.

Examples of beads can include, but are not limited to, streptavidin beads, agarose beads, magnetic beads, Dynabeads®, MACS® microbeads, antibody conjugated beads (e.g., anti-immunoglobulin microbeads), protein A conjugated beads, protein G conjugated beads, protein A/G conjugated beads, protein L conjugated beads, oligo(dT) conjugated beads, silica beads, silica-like beads, anti-biotin microbeads, anti-fluorochrome microbeads, and BcMag™ Carboxyl-Terminated Magnetic Beads.

A bead can be associated with (e.g. impregnated with) quantum dots or fluorescent dyes to make it fluorescent in one fluorescence optical channel or multiple optical channels. A bead can be associated with iron oxide or chromium oxide to make it paramagnetic or ferromagnetic. Beads can be identifiable. For example, a bead can be imaged using a camera. A bead can have a detectable code associated with the bead. For example, a bead can comprise a stochastic barcode. A bead can change size, for example due to swelling in an organic or inorganic solution. A bead can be hydrophobic. A bead can be hydrophilic. A bead can be biocompatible.

A solid support (e.g., bead) can be visualized. The solid support can comprise a visualizing tag (e.g., fluorescent dye). A solid support (e.g., bead) can be etched with an identifier (e.g., a number). The identifier can be visualized through imaging the beads.

### Substrates and Microwell Array

As used herein, a substrate can refer to a type of solid support. A substrate can refer to a solid support that can comprise stochastic barcodes of the disclosure. A substrate can, for example, comprise a plurality of microwells. For example, a substrate can be a well array comprising two or more microwells. In some embodiments, a microwell can comprise a small reaction chamber of defined volume. In some embodiments, a microwell can entrap one or more cells. In some embodiments, a microwell can entrap only one cell. In some embodiments, a microwell can entrap one or more solid supports. In some embodiments, a microwell can entrap only one solid support. In some embodiments, a microwell entraps a single cell and a single solid support (e.g., bead).

### Methods of Stochastic Barcoding

The disclosure provides for methods for estimating the number of distinct targets at distinct locations in a physical sample (e.g., tissue, organ, tumor, cell). The methods can comprise placing the stochastic barcodes in close proximity with the sample, lysing the sample, associating distinct targets with the stochastic barcodes, amplifying the targets and/or digitally counting the targets. The method can further comprise analyzing and/or visualizing the information obtained from the spatial labels on the stochastic barcodes. In some embodiments, a method comprises visualizing the plurality of targets in the sample. Mapping the plurality of targets onto the map of the sample can include generating a two dimensional map or a three dimensional map of the sample. The two dimensional map and the three dimensional map can be generated prior to or after stochastically barcoding the plurality of targets in the sample. Visualizing the plurality of targets in the sample can include mapping the plurality of targets onto a map of the sample. Mapping the plurality of targets onto the map of the sample can include generating a two dimensional map or a three dimensional map of the sample. The two dimensional map and the three dimensional map can be generated prior to or after stochastically barcoding the plurality of targets in the sample, in some embodiments, the two dimensional map and the three dimensional map can be generated before or after lysing the sample. Lysing the sample before or after generating the two dimensional map or the three dimensional map can include heating the sample, contacting the sample with a detergent, changing the pH of the sample, or any combination thereof.

In some embodiments, stochastically barcoding the plurality of targets comprises hybridizing a plurality of stochastic barcodes with a plurality of targets to create stochastically barcoded targets. Stochastically barcoding the plurality of targets can comprise generating an indexed library of the stochastically barcoded targets. Generating an indexed library of the stochastically barcoded targets can be performed with a solid support comprising the plurality of stochastic barcodes.

### Contacting a Sample and a Stochastic Barcode

The disclosure provides for methods for contacting a sample (e.g., cells) to a substrate of the disclosure. A sample comprising, for example, a cell, organ, or tissue thin section, can be contacted to stochastic barcodes. The cells can be contacted, for example, by gravity flow wherein the cells can settle and create a monolayer. The sample can be a tissue thin section. The thin section can be placed on the substrate. The sample can be one-dimensional (e.g., form a planar surface). The sample (e.g., cells) can be spread across the substrate, for example, by growing/culturing the cells on the substrate.

When stochastic barcodes are in close proximity to targets, the targets can hybridize to the stochastic barcode. The stochastic barcodes can be contacted at a non-depletable ratio such that each distinct target can associate with a distinct stochastic barcode of the disclosure. To ensure efficient association between the target and the stochastic barcode, the targets can be crosslinked to the stochastic barcode.

### Cell Lysis

Following the distribution of cells and stochastic barcodes, the cells can be lysed to liberate the target molecules. Cell lysis can be accomplished by any of a variety of means, for example, by chemical or biochemical means, by osmotic shock, or by means of thermal lysis, mechanical lysis, or optical lysis. Cells can be lysed by addition of a cell lysis buffer comprising a detergent (e.g. SDS, Li dodecyl sulfate, Triton X-100, Tween-20, or NP-40), an organic solvent (e.g. methanol or acetone), or digestive enzymes (e.g. proteinase K, pepsin, or trypsin), or any combination thereof. To increase the association of a target and a stochastic barcode, the rate of the diffusion of the target molecules can be altered by for example, reducing the temperature and/or increasing the viscosity of the lysate.

In some embodiments, the sample can be lysed using a filter paper. The filter paper can be soaked with a lysis buffer on top of the filter paper. The filter paper can be applied to the sample with pressure which can facilitate lysis of the sample and hybridization of the targets of the sample to the substrate.

In some embodiments, lysis can be performed by mechanical lysis, heat lysis, optical lysis, and/or chemical lysis. Chemical lysis can include the use of digestive enzymes such as proteinase K, pepsin, and trypsin. Lysis can be performed by the addition of a lysis buffer to the substrate. A lysis buffer can comprise Tris HCl. A lysis buffer can comprise at least about 0.01, 0.05, 0.1, 0.5, or 1 M or more Tris HCl. A lysis buffer can comprise at most about 0.01, 0.05, 0.1, 0.5, or 1 M or more Tris HCL. A lysis buffer can comprise about 0.1 M Tris HCl. The pH of the lysis buffer can be at least about 1, 2, 3, 4, 5, 6, 7, 8, 9, or 10 or more. The pH of the lysis buffer can be at most about 1, 2, 3, 4, 5, 6, 7, 8, 9, or 10 or more. In some embodiments, the pH of the lysis buffer is about 7.5. The lysis buffer can comprise a salt (e.g., LiCl). The concentration of salt in the lysis buffer can be at least about 0.1, 0.5, or 1 M or more. The concentration of salt in the lysis buffer can be at most about 0.1, 0.5, or 1 M or more. In some embodiments, the concentration of salt in the lysis buffer is about 0.5M. The lysis buffer can comprise a detergent (e.g., SDS, Li dodecyl sulfate, triton X, tween, NP-40). The concentration of the detergent in the lysis buffer can be at least about 0.0001%, 0.0005%, 0.001%, 0.005%, 0.01%, 0.05%, 0.1%, 0.5%, 1%, 2%, 3%, 4%, 5%, 6%, or 7% or more. The concentration of the detergent in the lysis buffer can be at most about 0.0001%, 0.0005%, 0.001%, 0.005%, 0.01%, 0.05%, 0.1%, 0.5%, 1%, 2%, 3%, 4%, 5%, 6%, or 7% or more. In some embodiments, the concentration of the detergent in the lysis buffer is about 1% Li dodecyl sulfate. The time used in the method for lysis can be dependent on the amount of detergent used. In some embodiments, the more detergent used, the less time needed for lysis. The lysis buffer can comprise a chelating agent (e.g., EDTA, EGTA). The concentration of a chelating agent in the lysis buffer can be at least about 1, 5, 10, 15, 20, 25, or 30 mM or more. The concentration of a chelating agent in the lysis buffer can be at most about 1, 5, 10, 15, 20, 25, or 30 mM or more. In some embodiments, the concentration of chelating agent in the lysis buffer is about 10 mM. The lysis buffer can comprise a reducing reagent (e.g., beta-mercaptoethanol, DTT). The concentration of the reducing reagent in the lysis buffer can be at least about 1, 5, 10, 15, or 20 mM or more. The concentration of the reducing reagent in the lysis buffer can be at most about 1, 5, 10, 15, or 20 mM or more. In some embodiments, the concentration of reducing reagent in the lysis buffer is about 5 mM. In some embodiments, a lysis buffer can comprise about 0.1M TrisHCl, about pH 7.5, about 0.5M LiCl, about 1% lithium dodecyl sulfate, about 10 mM EDTA, and about 5 mM DTT.

Lysis can be performed at a temperature of about 4, 10, 15, 20, 25, or 30° C. Lysis can be performed for about 1, 5, 10, 15, or 20 or more minutes. A lysed cell can comprise at least about 100000, 200000, 300000, 400000, 500000, 600000, or 700000 or more target nucleic acid molecules. A lysed cell can comprise at most about 100000, 200000, 300000, 400000, 500000, 600000, or 700000 or more target nucleic acid molecules.

### Attachment of Stochastic Barcodes to Target Nucleic Acid Molecules

Following lysis of the cells and release of nucleic acid molecules therefrom, the nucleic acid molecules can randomly associate with the stochastic barcodes of the co-localized solid support. Association can comprise hybridization of a stochastic barcode's target recognition region to a complementary portion of the target nucleic acid molecule (e.g., oligo(dT) of the stochastic barcode can interact with a poly(A) tail of a target). The assay conditions used for hybridization (e.g. buffer pH, ionic strength, temperature, etc.) can be chosen to promote formation of specific, stable hybrids. In some embodiments, the nucleic acid molecules released from the lysed cells can associate with the plurality of probes on the substrate (e.g., hybridize with the probes on the substrate). When the probes comprise oligo(dT), mRNA molecules can hybridize to the probes and be reverse transcribed. The oligo(dT) portion of the oligonucleotide can act as a primer for first strand synthesis of the cDNA molecule. For example, in a non-limiting example of stochastic barcoding illustrated in FIG. 2, at block 216, mRNA molecules can hybridize to stochastic barcodes on beads. For example, single-stranded nucleotide fragments can hybridize to the target-binding regions of stochastic barcodes.

Attachment can further comprise ligation of a stochastic barcode's target recognition region and a portion of the target nucleic acid molecule. For example, the target binding region can comprise a nucleic acid sequence that can be capable of specific hybridization to a restriction site overhang (e.g. an EcoRI sticky-end overhang). The assay procedure can further comprise treating the target nucleic acids with a restriction enzyme (e.g. EcoRI) to create a restriction site overhang. The stochastic barcode can then be ligated to any nucleic acid molecule comprising a sequence complementary to the restriction site overhang. A ligase (e.g., T4 DNA ligase) can be used to join the two fragments.

For example, in a non-limiting example of stochastic barcoding illustrated in FIG. 2, at block 220, the labeled targets from a plurality of cells (or a plurality of samples) (e.g., target-barcode molecules) can be subsequently pooled, for example, into a tube. The labeled targets can be pooled by, for example, retrieving the stochastic barcodes and/or the beads to which the target-barcode molecules are attached.

The retrieval of solid support-based collections of attached target-barcode molecules can be implemented by use of magnetic beads and an externally-applied magnetic field. Once the target-barcode molecules have been pooled, all further processing can proceed in a single reaction vessel. Further processing can include, for example, reverse transcription reactions, amplification reactions, cleavage reactions, dissociation reactions, and/or nucleic acid extension reactions. Further processing reactions can be performed within the microwells, that is, without first pooling the labeled target nucleic acid molecules from a plurality of cells.

### Reverse Transcription

The disclosure provides for a method to create a stochastic target-barcode conjugate using reverse transcription (e.g., at block 224 of FIG. 2). The stochastic target-barcode conjugate can comprise the stochastic barcode and a complementary sequence of all or a portion of the target nucleic acid (i.e. a stochastically barcoded cDNA molecule). Reverse transcription of the associated RNA molecule can occur by the addition of a reverse transcription primer along with the reverse transcriptase. The reverse transcription primer can be an oligo(dT) primer, a random hexanucleotide primer, or a target-specific oligonucleotide primer. Oligo (dT) primers can be, or can be about, 12-18 nucleotides in length and bind to the endogenous poly(A) tail at 3′ end of mammalian mRNA. Random hexanucleotide primers can bind to mRNA at a variety of complementary sites. Target-specific oligonucleotide primers typically selectively prime the mRNA of interest.

In some embodiments, reverse transcription of the labeled-RNA molecule can occur by the addition of a reverse transcription primer. In some embodiments, the reverse transcription primer is an oligo(dT) primer, random hexanucleotide primer, or a target-specific oligonucleotide primer. Generally, oligo(dT) primers are 12-18 nucleotides in length and bind to the endogenous poly(A)+ tail at 3′ end of mammalian mRNA. Random hexanucleotide primers can bind to mRNA at a variety of complementary sites. Target-specific oligonucleotide primers typically selectively prime the mRNA of interest.

Reverse transcription can occur repeatedly to produce multiple labeled-cDNA molecules. The methods disclosed herein can comprise conducting at least about 1, 2, 3, 4, 5, 6, 7, 8, 9, 10, 11, 12, 13, 14, 15, 16, 17, 18, 19, or 20 reverse transcription reactions. The method can comprise conducting at least about 25, 30, 35, 40, 45, 50, 55, 60, 65, 70, 75, 80, 85, 90, 95, or 100 reverse transcription reactions.

### Amplification

One or more nucleic acid amplification reactions (e.g., at block 228 of FIG. 2) can be performed to create multiple copies of the labeled target nucleic acid molecules. Amplification can be performed in a multiplexed manner, wherein multiple target nucleic acid sequences are amplified simultaneously. The amplification reaction can be used to add sequencing adaptors to the nucleic acid molecules. The amplification reactions can comprise amplifying at least a portion of a sample label, if present. The amplification reactions can comprise amplifying at least a portion of the cellular and/or molecular label. The amplification reactions can comprise amplifying at least a portion of a sample tag, a cell label, a spatial label, a molecular label, a target nucleic acid, or a combination thereof. The amplification reactions can comprise amplifying 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 100%, or a range or a number between any two of these values, of the plurality of nucleic acids. The method can further comprise conducting one or more cDNA synthesis reactions to produce one or more cDNA copies of target-barcode molecules comprising a sample label, a cell label, a spatial label, and/or a molecular label.

In some embodiments, amplification can be performed using a polymerase chain reaction (PCR). As used herein, PCR can refer to a reaction for the in vitro amplification of specific DNA sequences by the simultaneous primer extension of complementary strands of DNA. As used herein, PCR can encompass derivative forms of the reaction, including but not limited to, RT-PCR, real-time PCR, nested PCR, quantitative PCR, multiplexed PCR, digital PCR, and assembly PCR.

Amplification of the labeled nucleic acids can comprise non-PCR based methods. Examples of non-PCR based methods include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. Other non-PCR-based amplification methods include multiple cycles of DNA-dependent RNA polymerase-driven RNA transcription amplification or RNA-directed DNA synthesis and transcription to amplify DNA or RNA targets, a ligase chain reaction (LCR), and a Qβ replicase (Qβ) method, use of palindromic probes, strand displacement amplification, oligonucleotide-driven amplification using a restriction endonuclease, an amplification method in which a primer is hybridized to a nucleic acid sequence and the resulting duplex is cleaved prior to the extension reaction and amplification, strand displacement amplification using a nucleic acid polymerase lacking 5′ exonuclease activity, rolling circle amplification, and ramification extension amplification (RAM). In some embodiments, the amplification does not produce circularized transcripts.

In some embodiments, the methods disclosed herein further comprise conducting a polymerase chain reaction on the labeled nucleic acid (e.g., labeled-RNA, labeled-DNA, labeled-cDNA) to produce a stochastically labeled-amplicon. The labeled-amplicon can be double-stranded molecule. The double-stranded molecule can comprise a double-stranded RNA molecule, a double-stranded DNA molecule, or a RNA molecule hybridized to a DNA molecule. One or both of the strands of the double-stranded molecule can comprise a sample label, a spatial label, a cell label, and/or a molecular label. The stochastically labeled-amplicon can be a single-stranded molecule. The single-stranded molecule can comprise DNA, RNA, or a combination thereof. The nucleic acids of the disclosure can comprise synthetic or altered nucleic acids.

Amplification can comprise use of one or more non-natural nucleotides. Non-natural nucleotides can comprise photolabile or triggerable nucleotides. Examples of non-natural nucleotides can include, but are not limited to, peptide nucleic acid (PNA), morpholino and locked nucleic acid (LNA), as well as glycol nucleic acid (GNA) and threose nucleic acid (TNA). Non-natural nucleotides can be added to one or more cycles of an amplification reaction. The addition of the non-natural nucleotides can be used to identify products as specific cycles or time points in the amplification reaction.

Conducting the one or more amplification reactions can comprise the use of one or more primers. The one or more primers can comprise, for example, 1, 2, 3, 4, 5, 6, 7, 8, 9, 10, 11, 12, 13, 14, or 15 or more nucleotides. The one or more primers can comprise at least 1, 2, 3, 4, 5, 6, 7, 8, 9, 10, 11, 12, 13, 14, or 15 or more nucleotides. The one or more primers can comprise less than 12-15 nucleotides. The one or more primers can anneal to at least a portion of the plurality of stochastically labeled targets. The one or more primers can anneal to the 3′ end or 5′ end of the plurality of stochastically labeled targets. The one or more primers can anneal to an internal region of the plurality of stochastically labeled targets. The internal region can be at least about 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from 3′ ends the plurality of stochastically labeled targets. The one or more primers can comprise a fixed panel of primers. The one or more primers can comprise at least one or more custom primers. The one or more primers can comprise at least one or more control primers. The one or more primers can comprise at least one or more gene-specific primers.

The one or more primers can comprise a universal primer. The universal primer can anneal to a universal primer binding site. The one or more custom primers can anneal to a first sample label, a second sample label, a spatial label, a cell label, a molecular label, a target, or any combination thereof. The one or more primers can comprise a universal primer and a custom primer. The custom primer can be designed to amplify one or more targets. The targets can comprise a subset of the total nucleic acids in one or more samples. The targets can comprise a subset of the total stochastically labeled targets in one or more samples. The one or more primers can comprise at least 96 or more custom primers. The one or more primers can comprise at least 960 or more custom primers. The one or more primers can comprise at least 9600 or more custom primers. The one or more custom primers can anneal to two or more different labeled nucleic acids. The two or more different labeled nucleic acids can correspond to one or more genes.

Any amplification scheme can be used in the methods of the present disclosure. For example, in one scheme, the first round PCR can amplify molecules attached to the bead using a gene specific primer and a primer against the universal Illumina sequencing primer 1 sequence. The second round of PCR can amplify the first PCR products using a nested gene specific primer flanked by Illumina sequencing primer 2 sequence, and a primer against the universal Illumina sequencing primer 1 sequence. The third round of PCR adds P5 and P7 and sample index to turn PCR products into an Illumina sequencing library. Sequencing using 150 bp×2 sequencing can reveal the cell label and molecular label on read 1, the gene on read 2, and the sample index on index 1 read.

In some embodiments, nucleic acids can be removed from the substrate using chemical cleavage. For example, a chemical group or a modified base present in a nucleic acid can be used to facilitate its removal from a solid support. For example, an enzyme can be used to remove a nucleic acid from a substrate. For example, a nucleic acid can be removed from a substrate through a restriction endonucelase digestion. For example, treatment of a nucleic acid containing a dUTP or ddUTP with uracil-d-glycosylase (UDG) can be used to remove a nucleic acid from a substrate. For example, a nucleic acid can be removed from a substrate using an enzyme that performs nucleotide excision, such as a base excision repair enzyme, such as an apurinic/apyrimidinic (AP) endonuclease. In some embodiments, a nucleic acid can be removed from a substrate using a photocleavable group and light. In some embodiments, a cleavable linker can be used to remove a nucleic acid from the substrate. For example, the cleavable linker can comprise at least one of biotin/avidin, biotin/streptavidin, biotin/neutravidin, Ig-protein A, a photo-labile linker, acid or base labile linker group, or an aptamer.

When the probes are gene-specific, the molecules can hybridize to the probes and be reverse transcribed and/or amplified. In some embodiments, after the nucleic acid has been synthesized (e.g., reverse transcribed), it can be amplified. Amplification can be performed in a multiplex manner, wherein multiple target nucleic acid sequences are amplified simultaneously. Amplification can add sequencing adaptors to the nucleic acid.

In some embodiments, amplification can be performed on the substrate, for example, with bridge amplification. cDNAs can be homopolymer tailed in order to generate a compatible end for bridge amplification using oligo(dT) probes on the substrate. In bridge amplification, the primer that is complementary to the 3′ end of the template nucleic acid can be the first primer of each pair that is covalently attached to the solid particle. When a sample containing the template nucleic acid is contacted with the particle and a single thermal cycle is performed, the template molecule can be annealed to the first primer and the first primer is elongated in the forward direction by addition of nucleotides to form a duplex molecule consisting of the template molecule and a newly formed DNA strand that is complementary to the template. In the heating step of the next cycle, the duplex molecule can be denatured, releasing the template molecule from the particle and leaving the complementary DNA strand attached to the particle through the first primer. In the annealing stage of the annealing and elongation step that follows, the complementary strand can hybridize to the second primer, which is complementary to a segment of the complementary strand at a location removed from the first primer. This hybridization can cause the complementary strand to form a bridge between the first and second primers secured to the first primer by a covalent bond and to the second primer by hybridization. In the elongation stage, the second primer can be elongated in the reverse direction by the addition of nucleotides in the same reaction mixture, thereby converting the bridge to a double-stranded bridge. The next cycle then begins, and the double-stranded bridge can be denatured to yield two single-stranded nucleic acid molecules, each having one end attached to the particle surface via the first and second primers, respectively, with the other end of each unattached. In the annealing and elongation step of this second cycle, each strand can hybridize to a further complementary primer, previously unused, on the same particle, to form new single-strand bridges. The two previously unused primers that are now hybridized elongate to convert the two new bridges to double-strand bridges.

The amplification reactions can comprise amplifying at least, or at least about, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, or 100% of the plurality of nucleic acids.

Amplification of the labeled nucleic acids can comprise PCR-based methods or non-PCR based methods. Amplification of the labeled nucleic acids can comprise exponential amplification of the labeled nucleic acids. Amplification of the labeled nucleic acids can comprise linear amplification of the labeled nucleic acids. Amplification can be performed by polymerase chain reaction (PCR). PCR can refer to a reaction for the in vitro amplification of specific DNA sequences by the simultaneous primer extension of complementary strands of DNA. PCR can encompass derivative forms of the reaction, including but not limited to, RT-PCR, real-time PCR, nested PCR, quantitative PCR, multiplexed PCR, digital PCR, suppression PCR, semi-suppressive PCR and assembly PCR.

In some embodiments, amplification of the labeled nucleic acids comprises non-PCR based methods. Examples of non-PCR based methods include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. Other non-PCR-based amplification methods include multiple cycles of DNA-dependent RNA polymerase-driven RNA transcription amplification or RNA-directed DNA synthesis and transcription to amplify DNA or RNA targets, a ligase chain reaction (LCR), a Qβ replicase (Qβ), use of palindromic probes, strand displacement amplification, oligonucleotide-driven amplification using a restriction endonuclease, an amplification method in which a primer is hybridized to a nucleic acid sequence and the resulting duplex is cleaved prior to the extension reaction and amplification, strand displacement amplification using a nucleic acid polymerase lacking 5′ exonuclease activity, rolling circle amplification, and/or ramification extension amplification (RAM).

In some embodiments, the methods disclosed herein further comprise conducting a nested polymerase chain reaction on the amplified amplicon (e.g., target). The amplicon can be double-stranded molecule. The double-stranded molecule can comprise a double-stranded RNA molecule, a double-stranded DNA molecule, or a RNA molecule hybridized to a DNA molecule. One or both of the strands of the double-stranded molecule can comprise a sample tag or molecular identifier label. Alternatively, the amplicon can be a single-stranded molecule. The single-stranded molecule can comprise DNA, RNA, or a combination thereof. The nucleic acids of the present invention can comprise synthetic or altered nucleic acids.

In some embodiments, the method comprises repeatedly amplifying the labeled nucleic acid to produce multiple amplicons. The methods disclosed herein can comprise conducting at least, or at least about, 1, 2, 3, 4, 5, 6, 7, 8, 9, 10, 11, 12, 13, 14, 15, 16, 17, 18, 19, or 20 amplification reactions. Alternatively, the method comprises conducting at least, or at least about, 25, 30, 35, 40, 45, 50, 55, 60, 65, 70, 75, 80, 85, 90, 95, or 100 amplification reactions.

Amplification can further comprise adding one or more control nucleic acids to one or more samples comprising a plurality of nucleic acids. Amplification can further comprise adding one or more control nucleic acids to a plurality of nucleic acids. The control nucleic acids can comprise a control label.

Amplification can comprise use of one or more non-natural nucleotides. Non-natural nucleotides can comprise photolabile and/or triggerable nucleotides. Examples of non-natural nucleotides include, but are not limited to, peptide nucleic acid (PNA), morpholino and locked nucleic acid (LNA), as well as glycol nucleic acid (GNA) and threose nucleic acid (TNA). Non-natural nucleotides can be added to one or more cycles of an amplification reaction. The addition of the non-natural nucleotides can be used to identify products as specific cycles or time points in the amplification reaction.

Conducting the one or more amplification reactions can comprise the use of one or more primers. The one or more primers can comprise one or more oligonucleotides. The one or more oligonucleotides can comprise at least about 7-9 nucleotides. The one or more oligonucleotides can comprise less than 12-15 nucleotides. The one or more primers can anneal to at least a portion of the plurality of labeled nucleic acids. The one or more primers can anneal to the 3′ end and/or 5′ end of the plurality of labeled nucleic acids. The one or more primers can anneal to an internal region of the plurality of labeled nucleic acids. The internal region can be at least or at least about, 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from 3′ ends the plurality of labeled nucleic acids. The one or more primers can comprise a fixed panel of primers. The one or more primers can comprise at least one or more custom primers. The one or more primers can comprise at least one or more control primers. The one or more primers can comprise at least one or more housekeeping gene primers. The one or more primers can comprise a universal primer. The universal primer can anneal to a universal primer binding site. The one or more custom primers can anneal to the first sample tag, the second sample tag, the molecular identifier label, the nucleic acid or a product thereof. The one or more primers can comprise a universal primer and a custom primer. The custom primer can be designed to amplify one or more target nucleic acids. The target nucleic acids can comprise a subset of the total nucleic acids in one or more samples. In some embodiments, the primers are the probes attached to the array of the disclosure.

In some embodiments, stochastically barcoding the plurality of targets in the sample further comprises generating an indexed library of the stochastically barcoded fragments. The molecular labels of different stochastic barcodes can be different from one another. Generating an indexed library of the stochastically barcoded targets includes generating a plurality of indexed polynucleotides from the plurality of targets in the sample. For example, for an indexed library of the stochastically barcoded targets comprising a first indexed target and a second indexed target, the label region of the first indexed polynucleotide can differ from the label region of the second indexed polynucleotide by, by about, by at least, or by at most, 1, 2, 3, 4, 5, 6, 7, 8, 9, 10, 20, 30, 40, 50, or a number or a range between any two of these values, nucleotides. In some embodiments, generating an indexed library of the stochastically barcoded targets includes contacting a plurality of targets, for example mRNA molecules, with a plurality of oligonucleotides including a poly(T) region and a label region; and conducting a first strand synthesis using a reverse transcriptase to produce single-strand labeled cDNA molecules each comprising a cDNA region and a label region, wherein the plurality of targets includes at least two mRNA molecules of different sequences and the plurality of oligonucleotides includes at least two oligonucleotides of different sequences. Generating an indexed library of the stochastically barcoded targets can further comprise amplifying the single-strand labeled cDNA molecules to produce double-strand labeled cDNA molecules; and conducting nested PCR on the double-strand labeled cDNA molecules to produce labeled amplicons. In some embodiments, the method can include generating an adaptor-labeled amplicon.

Stochastic barcoding can use nucleic acid barcodes or tags to label individual nucleic acid (e.g., DNA or RNA) molecules. In some embodiments, it involves adding DNA barcodes or tags to cDNA molecules as they are generated from mRNA. Nested PCR can be performed to minimize PCR amplification bias. Adaptors can be added for sequencing using, for example, next generation sequencing (NGS). The sequencing results can be used to determine cell labels, molecular labels, and sequences of nucleotide fragments of the one or more copies of the targets, for example at block 232 of FIG. 2.

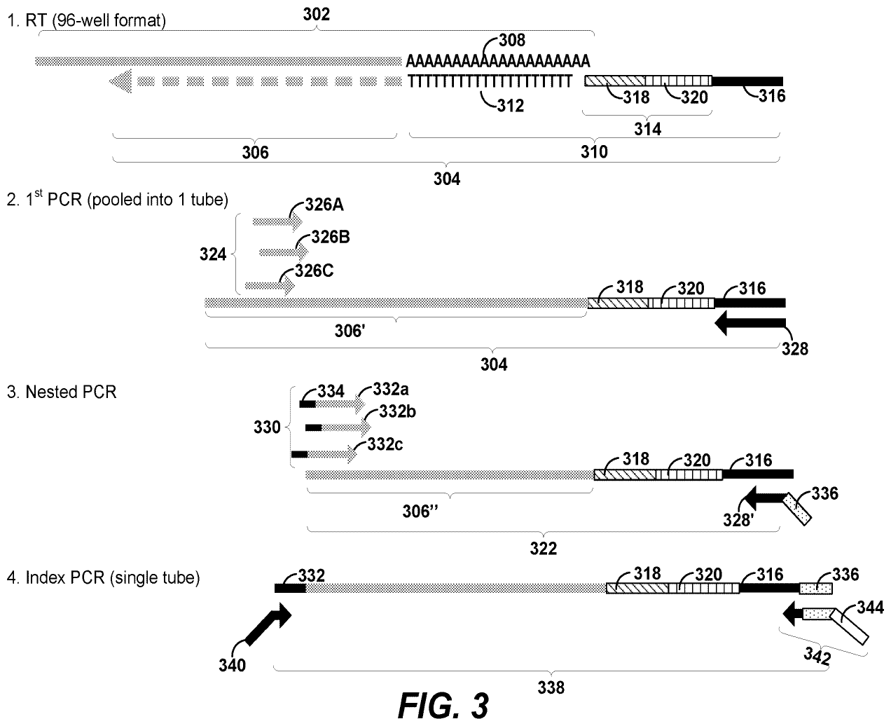

FIG. 3 is a schematic illustration showing a non-limiting exemplary process of generating an indexed library of the stochastically barcoded targets, for example mRNAs. As shown in step 1, the reverse transcription process can encode each mRNA molecule with a unique molecular label, a cell label, and a universal PCR site. In particular, RNA molecules 302 can be reverse transcribed to produce labeled cDNA molecules 304, including a cDNA region 306, by the stochastic hybridization of a set of molecular identifier labels 310 to the poly(A) tail region 308 of the RNA molecules 302. Each of the molecular identifier labels 310 can comprise a target-binding region, for example a poly(dT) region 312, a label region 314, and a universal PCR region 316.

In some embodiments, the cell label can include 3 to 20 nucleotides. In some embodiments, the molecular label can include 3 to 20 nucleotides. In some embodiments, each of the plurality of stochastic barcodes further comprises one or more of a universal label and a cell label, wherein universal labels are the same for the plurality of stochastic barcodes on the solid support and cell labels are the same for the plurality of stochastic barcodes on the solid support. In some embodiments, the universal label can include 3 to 20 nucleotides. In some embodiments, the cell label comprises 3 to 20 nucleotides.

In some embodiments, the label region 314 can include a molecular label 318 and a cell label 320. In some embodiments, the label region 314 can include one or more of a universal label, a dimension label, and a cell label. The molecular label 318 can be, can be about, can be at least, or can be at most, 1, 2, 3, 4, 5, 6, 7, 8, 9, 10, 20, 30, 40, 50, 60, 70, 80, 90, 100, or a number or a range between any of these values, of nucleotides in length. The cell label 320 can be, can be about, can be at least, or can be at most, 1, 2, 3, 4, 5, 6, 7, 8, 9, 10, 20, 30, 40, 50, 60, 70, 80, 90, 100, or a number or a range between any of these values, of nucleotides in length. The universal label can be, can be about, can be at least, or can be at most, 1, 2, 3, 4, 5, 6, 7, 8, 9, 10, 20, 30, 40, 50, 60, 70, 80, 90, 100, or a number or a range between any of these values, of nucleotides in length. Universal labels can be the same for the plurality of stochastic barcodes on the solid support and cell labels are the same for the plurality of stochastic barcodes on the solid support. The dimension label can be, can be about, can be at least, or can be at most 1, 2, 3, 4, 5, 6, 7, 8, 9, 10, 20, 30, 40, 50, 60, 70, 80, 90, 100, or a number or a range between any of these values, of nucleotides in length.

In some embodiments, the label region 314 can comprise, comprise about, comprise at least, or comprise at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any of these values, different labels, such as a molecular label 318 and a cell label 320. Each label can be, can be about, can be at least, or can be at most 1, 2, 3, 4, 5, 6, 7, 8, 9, 10, 20, 30, 40, 50, 60, 70, 80, 90, 100, or a number or a range between any of these values, of nucleotides in length. A set of molecular identifier labels 310 can contain, contain about, contain at least, or can be at most, 10, 20, 40, 50, 70, 80, 90, 10², 10³, 10⁴, 10⁵, 10⁶, 10⁷, 10⁸, 10⁹, 10¹⁰, 10¹¹, 10¹², 10¹³, 10¹⁴, 10¹⁵, 10²⁰, or a number or a range between any of these values, molecular identifier labels 310. And the set of molecular identifier labels 310 can, for example, each contain a unique label region 314. The labeled cDNA molecules 304 can be purified to remove excess molecular identifier labels 310. Purification can comprise Ampure bead purification.

As shown in step 2, products from the reverse transcription process in step 1 can be pooled into 1 tube and PCR amplified with a 1^{st} PCR primer pool and a 1^{st} universal PCR primer. Pooling is possible because of the unique label region 314. In particular, the labeled cDNA molecules 304 can be amplified to produce nested PCR labeled amplicons 322. Amplification can comprise multiplex PCR amplification. Amplification can comprise a multiplex PCR amplification with 96 multiplex primers in a single reaction volume. In some embodiments, multiplex PCR amplification can utilize, utilize about, utilize at least, or utilize at most, 10, 20, 40, 50, 70, 80, 90, 10², 10³, 10⁴, 10⁵, 10⁶, 10⁷, 10⁸, 10⁹, 10¹⁰, 10¹¹, 10¹², 10¹³, 10¹⁴, 10¹⁵, 10²⁰, or a number or a range between any of these values, multiplex primers in a single reaction volume. Amplification can comprise 1st PCR primer pool 324 of custom primers 326A-C targeting specific genes and a universal primer 328. The custom primers 326 can hybridize to a region within the cDNA portion 306′ of the labeled cDNA molecule 304. The universal primer 328 can hybridize to the universal PCR region 316 of the labeled cDNA molecule 304.

As shown in step 3 of FIG. 3, products from PCR amplification in step 2 can be amplified with a nested PCR primers pool and a 2^{nd} universal PCR primer. Nested PCR can minimize PCR amplification bias. In particular, the nested PCR labeled amplicons 322 can be further amplified by nested PCR. The nested PCR can comprise multiplex PCR with nested PCR primers pool 330 of nested PCR primers 332a-c and a 2^{nd} universal PCR primer 328′ in a single reaction volume. The nested PCR primer pool 328 can contain, contain about, contain at least, or contain at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any of these values, different nested PCR primers 330. The nested PCR primers 332 can contain an adaptor 334 and hybridize to a region within the cDNA portion 306″ of the labeled amplicon 322. The universal primer 328′ can contain an adaptor 336 and hybridize to the universal PCR region 316 of the labeled amplicon 322. Thus, step 3 produces adaptor-labeled amplicon 338. In some embodiments, nested PCR primers 332 and the 2nd universal PCR primer 328′ may not contain the adaptors 334 and 336. The adaptors 334 and 336 can instead be ligated to the products of nested PCR to produce adaptor-labeled amplicon 338.

As shown in step 4, PCR products from step 3 can be PCR amplified for sequencing using library amplification primers. In particular, the adaptors 334 and 336 can be used to conduct one or more additional assays on the adaptor-labeled amplicon 338. The adaptors 334 and 336 can be hybridized to primers 340 and 342. The one or more primers 340 and 342 can be PCR amplification primers. The one or more primers 340 and 342 can be sequencing primers. The one or more adaptors 334 and 336 can be used for further amplification of the adaptor-labeled amplicons 338. The one or more adaptors 334 and 336 can be used for sequencing the adaptor-labeled amplicon 338. The primer 342 can contain a plate index 344 so that amplicons generated using the same set of molecular identifier labels 318 can be sequenced in one sequencing reaction using next generation sequencing (NGS).

### Correction of Amplification Noise

After reverse transcribing mRNA molecules into cDNA molecules (e.g., in 224 of FIG. 2), stochastic barcodes not incorporated into cDNA molecules can be removed by, for example, Ampure bead purification. The removal method, for example Ampure bead purification, may not completely remove the stochastic barcodes that are not extended by reverse transcription to be incorporated into stochastically barcoded cDNA molecules. For example, 15%, 10%, 9%, 8%, 7%, 6%, 5%, 4%, 3%, 2%, 1%, 0.5%, 0.1%, or a range between any of these two values of stochastic barcodes that are not extended by reverse transcription to be incorporated into stochastically barcoded cDNA molecules may not be removed by Ampure bead purification. These unremoved stochastic barcodes can, for example, result in amplification noises during amplification of cDNA molecules (e.g., in 228 of FIG. 2) for sequencing. The stochastic barcodes between samples can be highly similar, for example the stochastic barcodes can be identical except for the sample labels and the molecular labels. Thus, PCR cross over can occur because these unremoved stochastic barcodes can hybridize to other nucleic acid molecules from other samples, for example the stochastic barcode regions of stochastically barcoded mRNA molecules, during PCR and can result in amplification noises after sequencing (e.g., in 232 of FIG. 2).

Disclosed herein are methods for determining the number of targets in one or more samples, where the methods comprise one or more steps for correcting amplification noises. In some embodiments, the method comprises: determining a pre-correction number of a target; determining an amplification noise; and removing the amplification noise from the pre-correction number of the targets to generate the estimated number of the target. In some embodiments, the method comprises: stochastically barcoding a plurality of targets in each of one or more samples using a plurality of oligonucleotides comprising stochastic barcodes to generate stochastically barcoded targets, wherein for each sample, each stochastic barcode comprises a molecular label and an identical sample label, wherein the molecular labels of at least two stochastic barcodes differ from one another by at least one nucleotide; contacting one or more defined barcoded primers with each of the one or more samples, wherein each of the one or more defined barcoded primers comprises a defined sample label and a defined molecular label, and wherein the defined sample labels can be variants of the sample labels; amplifying the stochastically barcoded targets and the one or more defined barcoded primers to generate a plurality of amplified stochastically barcoded targets and a plurality of amplified defined barcoded primers; and estimating the number of each of the plurality of targets, wherein estimating the number of each of the plurality of targets comprises: determining a pre-correction number of each of the plurality of targets using the molecular label; determining an amplification noise by determining the number of defined molecular labels with different sequences in the plurality of amplified defined barcoded primers; and removing the amplification noise from the pre-correction number of each of the plurality of targets to generate the estimated number of each of the plurality of targets. The method can be multiplexed.

### Determine Molecular Label Counts

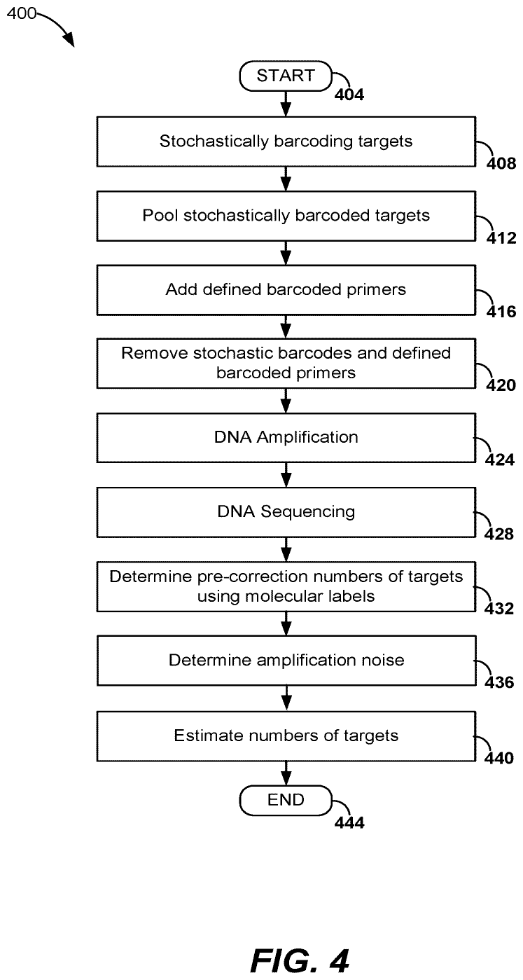

FIG. 4 is a flowchart showing a non-limiting exemplary embodiment of removing amplification noise caused by PCR crossover. The embodiment 400 starts at beginning block 404. At block 408, a plurality of targets in each of one or more samples can be stochastically barcoded using a plurality of oligonucleotides comprising stochastic barcodes to generate stochastically barcoded targets. In some embodiments, stochastically barcoding the plurality of targets comprises hybridizing the plurality of oligonucleotides comprising the stochastic barcodes with the plurality of targets to generate the stochastically barcoded targets. For each sample, a stochastic barcode can comprise a molecular label and an identical sample label. The molecular labels of at least two stochastic barcodes can differ from one another by at least one nucleotide.

At block 412, the stochastically barcoded targets can be optionally pooled, for example, into a tube to generate a pool of stochastically barcoded targets. At block 416, one or more defined barcoded primers can be contacted with each of the one or more samples. For example, if the stochastically barcoded targets have been pooled into a tube to generate a pool of stochastically barcoded target, then defined barcoded primers can be added to the pool of stochastically barcoded targets. As another example, if the stochastically barcoded targets have not been pooled into a tube, then defined barcoded primers can be added to one or more samples. The defined barcoded primers added to different samples can be the same or can be different, for example, defined barcoded primers for different genes. In some embodiments, contacting the one or more defined barcoded primers with each of the one or more samples comprises introducing the one or more defined barcoded primers at the same concentration of the plurality of oligonucleotides comprising the stochastic barcodes.

Defined barcoded primers can be variants of the stochastic barcodes. For example, a defined barcoded primer can have a corresponding stochastic barcode, where the defined barcoded primer is a variant of the corresponding stochastic barcode. The defined barcoded primer can have a different sequence in one or more regions as compared to the corresponding stochastic barcode. For example, a defined barcoded primer can comprise a defined sample label and a defined molecular label.

In some embodiments, the defined sample label of a defined barcoded primer can be a variant of the sample label of a stochastic barcode (e.g., the sample label of the corresponding stochastic barcode). For example, the defined sample label differs from the sample label in one, two, three, four, five, six, seven, eight, nine, ten, or a range between any two of these values, nucleotides. The defined sample label can be, for example, 5, 6, 7, 8, 9, 10, 11, 12, 13, 14, 15, 16, 17, 18 19, 20, or a range between any two of these values, nucleotides in length. The sample label can be 5, 6, 7, 8, 9, 10, 11, 12, 13, 14, 15, 16, 17, 18 19, 20, or a range between any two of these values, nucleotides in length. The defined sample label and the sample label, for example, a defined sample label of a defined barcoded primer and a sample label of the corresponding stochastic barcode, can have the same length or different length. For example, the defined sample label and the sample label can both be 8 nucleotides in length. In some embodiments, the sequence of the defined sample label and the sequence of the sample label can differ by one nucleotide. In some embodiments, the Hamming distance between the defined sample label and the sample label can be 2 or 4.

In some embodiments, the defined molecular label of a defined barcoded primer can be a variant of the molecular label of a stochastic barcode (e.g., the molecular label of the corresponding stochastic barcode). For example, the sequence of the defined molecular label and the sequence of the molecular label differ by one, two, three, four, five, six, seven, eight, nine, ten, or a range between any two of these values, nucleotides. The defined molecular label can be, for example, 5, 6, 7, 8, 9, 10, 11, 12, 13, 14, 15, 16, 17, 18 19, 20, or a range between any two of these values, nucleotides in length. The molecular label can be, for example, 5, 6, 7, 8, 9, 10, 11, 12, 13, 14, 15, 16, 17, 18 19, 20, or a range between any two of these values, nucleotides in length. The defined molecular label and the molecular label, for example a defined molecular label of a defined barcoded primer and a molecular label of the corresponding stochastic barcode, can have the same length. For example, the defined molecular label and the molecular label can both be 8 nucleotides in length. The defined molecular labels of different defined barcoded primers can be different from one another. The molecular labels of different stochastic barcodes can be different from one another. Some molecular labels of the stochastic barcodes and some defined molecular labels of the defined barcoded primers can have the same sequence.

In some embodiments, the one or more defined barcoded primers can comprise one or more types of defined barcoded primers. As disclosed herein, there can be different types of stochastic barcodes. In some embodiments, one type of defined barcoded primers can have a corresponding type of stochastic barcodes, where the one type of the defined barcoded primers is a variant of the corresponding type of stochastic barcodes. For example, one type of defined barcoded primers and the corresponding type of stochastic barcodes can both include poly(T) regions as their target binding region. Other types of defined barcoded primers and their corresponding types of stochastic barcodes can both include gene specific target binding regions. For example, one type of defined barcoded primers and the corresponding type of stochastic barcode can both include target binding regions for TOR alpha. As another example, another type of defined barcoded primer and its corresponding type of stochastic barcodes can include target binding regions for TOR alpha. In some embodiments, the defined sample labels of one type of defined barcoded primers and the sample labels of the corresponding type of stochastic barcodes can have different sequences.

In some embodiments, the defined sample labels of the same type of defined barcoded primers can have the same sequence. The defined molecular labels of the same type of defined barcoded primers can differ from one another by at least one nucleotide. The sample labels of different types of stochastic barcodes can have different sequences. Some defined molecular labels of different types of defined barcoded primers can have the same sequence. Different types of defined barcoded primers can have the same length. The lengths of different types of defined barcoded primers can be different. In some embodiments, the lengths of different types of defined barcoded primers can differ by, or by about, 1, 2, 3, 4, 5, 6, 7, 8, 9, 10, 11, 12, 13, 14, 15, 16, 17, 18, 19, 20, or a number or a range between any two of these values, nucleotides. In some embodiments, the lengths of different types of defined barcoded primers can differ by at least, or by at most, 1, 2, 3, 4, 5, 6, 7, 8, 9, 10, 11, 12, 13, 14, 15, 16, 17, 18, 19, or 20. The defined sample labels of different types of defined barcoded primers can have different sequences. The sample label and the defined sample label can have the same length.

The Hamming distance between the sample labels of the stochastic barcodes and the defined sample labels of the defined barcoded primers can be more than 0. In some embodiments, the Hamming distance between the sample labels of the stochastic barcodes and the defined sample labels of the defined barcoded primers can be, or be about, 1, 2, 3, 4, 5, 6, 7, 8, 9, 10, or a number or a range between any two of these values. In some embodiments, the Hamming distance between the sample labels of the stochastic barcodes and the defined sample labels of the defined barcoded primers can be at least, or at most, 1, 2, 3, 4, 5, 6, 7, 8, 9, 10. The stochastic barcodes and the one or more types of defined barcoded primers can have the same length or different lengths.

At block 420, the oligonucleotides comprising stochastic barcodes that are not incorporated into the stochastically barcoded targets, and the defined barcoded primers from each of the one or more samples can be removed. The percentage of unincorporated stochastic barcodes not removed can vary. In some embodiments, the percentage of unincorporated stochastic barcodes not removed can be, or be about, 10%, 20%, 30%, 40%, 50%, 60%, 70%, 80%, 90%, or a number or a range between any two of these values. In some embodiments, the percentage of unincorporated stochastic barcodes not removed can be at least, or at most, 10%, 20%, 30%, 40%, 50%, 60%, 70%, 80%, or 90%.

The percentage of the one or more defined barcoded primers not removed from each of the one or more samples can be substantially the same as the percentage of the plurality of oligonucleotides comprising the stochastic barcodes not removed from each of the one or more samples. In some embodiments, the percentage of the one or more defined barcoded primers not removed from each of the one or more samples and the percentage of the plurality of oligonucleotides comprising the stochastic barcodes not removed from each of the one or more samples can be within can be within 1%, 2%, 3%, 4%, 5%, 6%, 7%, 8%, 9%, 10%, 20%, 30%, 40%, 50%, or a number or a range between any two of these values, of each other.

At block 424, the stochastically barcoded targets and the one or more defined barcoded primers can be amplified to generate a plurality of amplified stochastically barcoded targets and a plurality of amplified defined barcoded primers. In some embodiments, amplifying the stochastically barcoded targets and the one or more defined barcoded primers to generate the plurality of amplified stochastically barcoded targets and the plurality of amplified defined barcoded primers comprises amplifying the stochastically barcoded targets and the one or more defined barcoded primers by polymerase chain reaction (PCR).

At block 428, the plurality of amplified stochastically barcoded targets and the plurality of amplified defined barcoded primers can be sequenced, by for example next generation sequencing (described in further details below). At block 432, a pre-correction number of each of the plurality of targets using the molecular label can be determined. In some embodiments, determining the pre-correction number of each of the plurality of targets using the molecular label comprises: determining sequences of molecular labels of the amplified stochastically barcoded targets; and counting the number of the molecular labels with different sequences. Determining the sequences of the molecular labels of the amplified stochastically barcoded targets can comprise sequencing some or all of the plurality of amplified stochastically barcoded targets. Sequencing some or all of the amplified stochastically barcoded targets can comprise generating sequences of the amplified stochastically barcoded targets. The read length of a sequence can vary. In some embodiments, the read length of a sequence can be, or be about, 50, 75, 100, 150, 200, 300, 400, 500, 600, 700, 800, 900, 1000, or a number or a range between any two of these values, bases. In some embodiments, the read length of a sequence can be, or be about, 50, 75, 100, 150, 200, 300, 400, 500, 600, 700, 800, 900, or 1000 bases.

At block 436, an amplification noise can be determined. The amplification noise can be determined by the number of defined molecular labels with different sequences in the plurality of amplified defined barcoded primers. In some embodiments, determining the number of defined molecular labels with different sequences in the plurality of amplified defined barcoded primers comprises: determining sequences of the defined molecular labels of the amplified defined barcoded primers; and counting the number of defined molecular labels with different sequences. Determining the sequences of the defined molecular labels of the amplified defined barcoded primers can comprise sequencing some or all of the plurality of amplified defined barcoded primers. Sequencing some or all of the plurality of amplified defined barcoded primers can comprise generating sequences of the amplified defined barcoded primers. The read length of a sequence can vary. In some embodiments, the read length of a sequence can be, or be about, 50, 75, 100, 150, 200, 300, 400, 500, 600, 700, 800, 900, 1000, or a number or a range between any two of these values, bases. In some embodiments, the read length of a sequence can be, or be about, 50, 75, 100, 150, 200, 300, 400, 500, 600, 700, 800, 900, or 1000 bases.

In some embodiments, determining the amplification noise comprises determining the number of defined molecular labels with different sequences in the plurality of amplified stochastically barcoded targets comprises determining the number of defined molecular labels in the plurality of amplified defined barcoded primers with different sequences for each type of amplified defined barcoded primers. Determining the amplification noise can comprise determining the number of defined molecular labels with different sequences in the plurality of amplified stochastically barcoded can comprise determining the average number of defined molecular labels in the plurality of amplified defined barcoded primers with different sequences for different types of amplified defined barcoded primers.

In some embodiments, determining the amplification noise comprises determining the number of defined molecular labels with different sequences in the plurality of amplified stochastically barcoded targets and the plurality of amplified defined barcoded primers can comprise determining the maximum number of defined molecular labels in the plurality of amplified stochastically barcoded targets and the plurality of amplified defined barcoded primers with different sequences for different types of amplified defined barcoded primers. The amplification noise can comprise noise caused by PCR crossover.

At block 440, the number of each of the plurality of targets can be estimated. Estimating the number of each of the plurality of targets can comprise: removing the amplification noise from the pre-correction number of each of the plurality of targets to generate the estimated number of each of the plurality of targets. The embodiment 400 ends at block 444.

The embodiment 400 can be used for determining amplification noise. In some embodiments, stochastically barcoding a plurality of targets using a plurality of oligonucleotides comprising stochastic barcodes to generate stochastically barcoded targets can comprise reverse transcribing the plurality targets in each of one or more samples using oligonucleotides comprising reverse transcription primers to generate reverse transcribed targets.

The embodiment 400 can be used, for example, for determining the number of targets in microwells of a microwell array. In some embodiments, stochastically barcoding a plurality of targets using a plurality of oligonucleotides comprising stochastic barcodes to generate stochastically barcoded targets can comprise stochastically barcoding a plurality of targets in each of one or more samples in microwells of a microwell array using a plurality of oligonucleotides comprising stochastic barcodes to generate stochastically barcoded targets. For each well, each stochastic barcode can comprise a molecular label and an identical sample label. The molecular labels of at least two stochastic barcodes can differ from one another by at least one nucleotide.

### Sequencing

In some embodiments, estimating the number of different stochastically barcoded targets can comprise determining the sequences of the labeled targets, the spatial label, the molecular label, the sample label, the cell label, or any product thereof (e.g. labeled-amplicons, or labeled-cDNA molecules). An amplified target can be subjected to sequencing. Determining the sequence of the stochastically barcoded target or any product thereof can comprise conducting a sequencing reaction to determine the sequence of at least a portion of a sample label, a spatial label, a cell label, a molecular label, at least a portion of the stochastically labeled target, a complement thereof, a reverse complement thereof, or any combination thereof.

Determination of the sequence of a stochastically barcoded target (e.g. amplified nucleic acid, labeled nucleic acid, cDNA copy of a labeled nucleic acid, etc.) can be performed using variety of sequencing methods including, but not limited to, sequencing by hybridization (SBH), sequencing by ligation (SBL), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads, wobble sequencing, multiplex sequencing, polymerized colony (POLONY) sequencing; nanogrid rolling circle sequencing (ROLONY), allele-specific oligo ligation assays (e.g., oligo ligation assay (OLA), single template molecule OLA using a ligated linear probe and a rolling circle amplification (RCA) readout, ligated padlock probes, or single template molecule OLA using a ligated circular padlock probe and a rolling circle amplification (RCA) readout), and the like.

In some embodiments, determining the sequence of the stochastically barcoded target or any product thereof comprises paired-end sequencing, nanopore sequencing, high-throughput sequencing, shotgun sequencing, dye-terminator sequencing, multiple-primer DNA sequencing, primer walking, Sanger dideoxy sequencing, Maxim-Gilbert sequencing, pyrosequencing, true single molecule sequencing, or any combination thereof. Alternatively, the sequence of the stochastically barcoded target or any product thereof can be determined by electron microscopy or a chemical-sensitive field effect transistor (chemFET) array.

High-throughput sequencing methods, such as cyclic array sequencing using platforms such as Roche 454, Illumina Solexa, ABI-SOLID, ION Torrent, Complete Genomics, Pacific Bioscience, Helicos, or the Polonator platform, can also be utilized. In some embodiment, sequencing can comprise MiSeq sequencing. In some embodiment, sequencing can comprise HiSeq sequencing.

The stochastically labeled targets can comprise nucleic acids representing from about 0.01% of the genes of an organism's genome to about 100% of the genes of an organism's genome. For example, about 0.01% of the genes of an organism's genome to about 100% of the genes of an organism's genome can be sequenced using a target complimentary region comprising a plurality of multimers by capturing the genes containing a complimentary sequence from the sample. In some embodiments, the stochastically barcoded targets comprise nucleic acids representing from about 0.01% of the transcripts of an organism's transcriptome to about 100% of the transcripts of an organism's transcriptome. For example, about 0.501% of the transcripts of an organism's transcriptome to about 100% of the transcripts of an organism's transcriptome can be sequenced using a target complimentary region comprising a poly(T) tail by capturing the mRNAs from the sample.

Determining the sequences of the spatial labels and the molecular labels of the plurality of the stochastic barcodes can include sequencing 0.00001%, 0.0001%, 0.001%, 0.01%, 0.1%, 1%, 2%, 3%, 4%, 5%, 6%, 7%, 8%, 9%, 10%, 20%, 30%, 40%, 50%, 60%, 70%, 80%, 90%, 99%, 100%, or a number or a range between any two of these values, of the plurality of stochastic barcodes. Determining the sequences of the labels of the plurality of stochastic barcodes, for example the sample labels, the spatial labels, and the molecular labels, can include sequencing 1, 10, 20, 30, 40, 50, 60, 70, 80, 90, 100, 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or a number or a range between any two of these values, of the plurality of stochastic barcodes. Sequencing some or all of the plurality of stochastic barcodes can include generating sequences with read lengths of, of about, of at least, or of at most, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, or a number or a range between any two of these values, of nucleotides or bases.

Sequencing can comprise sequencing at least or at least about 10, 20, 30, 40, 50, 60, 70, 80, 90, 100 or more nucleotides or base pairs of the stochastically barcoded targets. For example, sequencing can comprise generating sequencing data with sequences with read lengths of 50, 75, or 100, or more nucleotides by performing polymerase chain reaction (PCR) amplification on the plurality of stochastically barcoded targets. Sequencing can comprise sequencing at least or at least about 200, 300, 400, 500, 600, 700, 800, 900, 1,000 or more nucleotides or base pairs of the stochastically barcoded targets. Sequencing can comprise sequencing at least or at least about 1500, 2000, 3000, 4000, 5000, 6000, 7000, 8000, 9000, or 10000 or more nucleotides or base pairs of the stochastically barcoded targets.

Sequencing can comprise at least about 200, 300, 400, 500, 600, 700, 800, 900, 1,000 or more sequencing reads per run. In some embodiments, sequencing comprises sequencing at least or at least about 1500, 2000, 3000, 4000, 5000, 6000, 7000, 8000, 9000, or 10000 or more sequencing reads per run. Sequencing can comprise less than or equal to about 1,600,000,000 sequencing reads per run. Sequencing can comprise less than or equal to about 200,000,000 reads per run.

### Samples

In some embodiments, the plurality of targets can be comprised in one or more samples. A sample can comprise one or more cells, or nucleic acids from one or more cells. A sample can be, for example, a single cell or nucleic acids from a single cell. The one or more cells can be of one or more cell types. At least one of the one or more cell types can be brain cell, heart cell, cancer cell, circulating tumor cell, organ cell, epithelial cell, metastatic cell, benign cell, primary cell, circulatory cell, or any combination thereof.

A sample for use in the method of the disclosure can comprise one or more cells. A sample can refer to one or more cells. In some embodiments, the plurality of cells can include one or more cell types. At least one of the one or more cell types can be brain cell, heart cell, cancer cell, circulating tumor cell, organ cell, epithelial cell, metastatic cell, benign cell, primary cell, circulatory cell, or any combination thereof. In some embodiments, the cells are cancer cells excised from a cancerous tissue, for example, breast cancer, lung cancer, colon cancer, prostate cancer, ovarian cancer, pancreatic cancer, brain cancer, melanoma and non-melanoma skin cancers, and the like. In some embodiments, the cells are derived from a cancer but collected from a bodily fluid (e.g. circulating tumor cells). Non-limiting examples of cancers can include, adenoma, adenocarcinoma, squamous cell carcinoma, basal cell carcinoma, small cell carcinoma, large cell undifferentiated carcinoma, chondrosarcoma, and fibrosarcoma. The sample can include a tissue, a cell monolayer, fixed cells, a tissue section, or any combination thereof. The sample can include a biological sample, a clinical sample, an environmental sample, a biological fluid, a tissue, or a cell from a subject. The sample can be obtained from a human, a mammal, a dog, a rat, a mouse, a fish, a fly, a worm, a plant, a fungus, a bacterium, a virus, a vertebrate, or an invertebrate.

In some embodiments, the cells are cells that have been infected with virus and contain viral oligonucleotides. In some embodiments, the viral infection can be caused by a virus such as single-stranded (+ strand or “sense”) DNA viruses (e.g. parvoviruses), or double-stranded RNA viruses (e.g. reoviruses). In some embodiments, the cells are bacteria. These can include either gram-positive or gram-negative bacteria. In some embodiments, the cells are fungi. In some embodiments, the cells are protozoans or other parasites.

As used herein, the term “cell” can refer to one or more cells. In some embodiments, the cells are normal cells, for example, human cells in different stages of development, or human cells from different organs or tissue types. In some embodiments, the cells are non-human cells, for example, other types of mammalian cells (e.g. mouse, rat, pig, dog, cow, or horse). In some embodiments, the cells are other types of animal or plant cells. In other embodiments, the cells can be any prokaryotic or eukaryotic cells.

In some embodiments the cells are sorted prior to associating a cell with a bead. For example the cells can be sorted by fluorescence-activated cell sorting or magnetic-activated cell sorting, or more generally by flow cytometry. The cells can be filtered by size. In some embodiments a retentate contains the cells to be associated with the bead. In some embodiments the flow through contains the cells to be associated with the bead.

A sample can refer to a plurality of cells. The sample can refer to a monolayer of cells. The sample can refer to a thin section (e.g., tissue thin section). The sample can refer to a solid or semi-solid collection of cells that can be place in one dimension on an array.

### Devices

Disclosed herein are kits for determining the number of targets in one or more samples. In some embodiments, a kit comprises: a plurality of oligonucleotides comprising stochastic barcodes, wherein each stochastic barcode comprises a sample label and a molecular label, wherein the sample labels of at least two stochastic barcodes have different sequences, and wherein the molecular labels of stochastic barcodes with sample labels of the same sequence differ from one another by at least one nucleotide; defined barcoded primers, wherein each defined barcoded primer comprises a defined sample label and a defined molecular label, wherein the defined sample labels of defined barcoded primers can be variants of the sample labels of the stochastic barcodes, and wherein the Hamming distance between the sample label and the defined sample label is at least 2; and instructions for using the plurality oligonucleotides comprising the plurality of oligonucleotides and the defined barcoded primers.

### Flow Cells

The microwell array substrate can be packaged within a flow cell that provides for convenient interfacing with the rest of the fluid handling system and facilitates the exchange of fluids, e.g. cell and solid support suspensions, lysis buffers, rinse buffers, etc., that are delivered to the microwell array and/or emulsion droplet. Design features can include: (i) one or more inlet ports for introducing cell samples, solid support suspensions, or other assay reagents, (ii) one or more microwell array chambers designed to provide for uniform filling and efficient fluid-exchange while minimizing back eddies or dead zones, and (iii) one or more outlet ports for delivery of fluids to a sample collection point or a waste reservoir. The design of the flow cell can include a plurality of microarray chambers that interface with a plurality of microwell arrays such that one or more different cell samples can be processed in parallel. The design of the flow cell can further include features for creating uniform flow velocity profiles, i.e. “plug flow”, across the width of the array chamber to provide for more uniform delivery of cells and beads to the microwells, for example, by using a porous barrier located near the chamber inlet and upstream of the microwell array as a “flow diffuser”, or by dividing each array chamber into several subsections that collectively cover the same total array area, but through which the divided inlet fluid stream flows in parallel. In some embodiments, the flow cell can enclose or incorporate more than one microwell array substrate. In some embodiments, the integrated microwell array/flow cell assembly can constitute a fixed component of the system. In some embodiments, the microwell array/flow cell assembly can be removable from the instrument.

In general, the dimensions of fluid channels and the array chamber(s) in flow cell designs will be optimized to (i) provide uniform delivery of cells and beads to the microwell array, and (ii) to minimize sample and reagent consumption. In some embodiments, the width of fluid channels will be between 50 um and 20 mm. In other embodiments, the width of fluid channels can be at least 50 um, at least 100 um, at least 200 um, at least 300 um, at least 400 um, at least 500 um, at least 750 um, at least 1 mm, at least 2.5 mm, at least 5 mm, at least 10 mm, at least 20 mm, at least 50 mm, at least 100 mm, or at least 150 mm. In yet other embodiments, the width of fluid channels can be at most 150 mm, at most 100 mm, at most 50 mm, at most 20 mm, at most 10 mm, at most 5 mm, at most 2.5 mm, at most 1 mm, at most 750 um, at most 500 um, at most 400 um, at most 300 um, at most 200 um, at most 100 um, or at most 50 um. In one embodiment, the width of fluid channels is about 2 mm. The width of the fluid channels can fall within any range bounded by any of these values (e.g. from about 250 um to about 3 mm).

In some embodiments, the depth of the fluid channels will be between 50 um and 2 mm. In other embodiments, the depth of fluid channels can be at least 50 um, at least 100 um, at least 200 um, at least 300 um, at least 400 um, at least 500 um, at least 750 um, at least 1 mm, at least 1.25 mm, at least 1.5 mm, at least 1.75 mm, or at least 2 mm. In yet other embodiments, the depth of fluid channels can at most 2 mm, at most 1.75 mm, at most 1.5 mm, at most 1.25 mm, at most 1 mm, at most 750 um, at most 500 um, at most 400 um, at most 300 um, at most 200 um, at most 100 um, or at most 50 um. In one embodiment, the depth of the fluid channels is about 1 mm. The depth of the fluid channels can fall within any range bounded by any of these values (e.g. from about 800 um to about 1 mm).

Flow cells can be fabricated using a variety of techniques and materials known to those of skill in the art. In general, the flow cell will be fabricated as a separate part and subsequently either mechanically clamped or permanently bonded to the microwell array substrate. Examples of suitable fabrication techniques include conventional machining, CNC machining, injection molding, 3D printing, alignment and lamination of one or more layers of laser or die-cut polymer films, or any of a number of microfabrication techniques such as photolithography and wet chemical etching, dry etching, deep reactive ion etching, or laser micromachining. Once the flow cell part has been fabricated it can be attached to the microwell array substrate mechanically, e.g. by clamping it against the microwell array substrate (with or without the use of a gasket), or it can be bonded directly to the microwell array substrate using any of a variety of techniques (depending on the choice of materials used) known to those of skill in the art, for example, through the use of anodic bonding, thermal bonding, or any of a variety of adhesives or adhesive films, including epoxy-based, acrylic-based, silicone-based, UV curable, polyurethane-based, or cyanoacrylate-based adhesives.

Flow cells can be fabricated using a variety of materials known to those of skill in the art. In general, the choice of material used will depend on the choice of fabrication technique used, and vice versa. Examples of suitable materials include, but are not limited to, silicon, fused-silica, glass, any of a variety of polymers, e.g. polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COL), polyethylene terephthalate (PET), epoxy resins, metals (e.g. aluminum, stainless steel, copper, nickel, chromium, and titanium), a non-stick material such as teflon (PTFE), or a combination of these materials.

### Cartridges

In some embodiments of the system, the microwell array, with or without an attached flow cell, can be packaged within a consumable cartridge that interfaces with the instrument system. Design features of cartridges can include (i) one or more inlet ports for creating fluid connections with the instrument or manually introducing cell samples, bead suspensions, or other assay reagents into the cartridge, (ii) one or more bypass channels, i.e. for self-metering of cell samples and bead suspensions, to avoid overfilling or back flow, (iii) one or more integrated microwell array/flow cell assemblies, or one or more chambers within which the microarray substrate(s) are positioned, (iv) integrated miniature pumps or other fluid actuation mechanisms for controlling fluid flow through the device, (v) integrated miniature valves (or other containment mechanisms) for compartmentalizing pre-loaded reagents (for example, bead suspensions) or controlling fluid flow through the device, (vi) one or more vents for providing an escape path for trapped air, (vii) one or more sample and reagent waste reservoirs, (viii) one or more outlet ports for creating fluid connections with the instrument or providing a processed sample collection point, (ix) mechanical interface features for reproducibly positioning the removable, consumable cartridge with respect to the instrument system, and for providing access so that external magnets can be brought into close proximity with the microwell array, (x) integrated temperature control components or a thermal interface for providing good thermal contact with the instrument system, and (xi) optical interface features, e.g. a transparent window, for use in optical interrogation of the microwell array.

The cartridge can be designed to process more than one sample in parallel. The cartridge can further comprise one or more removable sample collection chamber(s) that are suitable for interfacing with stand-alone PCR thermal cyclers or sequencing instruments. The cartridge itself can be suitable for interfacing with stand-alone PCR thermal cyclers or sequencing instruments. The term “cartridge” as used in this disclosure can be meant to include any assembly of parts which contains the sample and beads during performance of the assay.

The cartridge can further comprise components that are designed to create physical or chemical barriers that prevent diffusion of (or increase path lengths and diffusion times for) large molecules in order to minimize cross-contamination between microwells. Examples of such barriers can include, but are not limited to, a pattern of serpentine channels used for delivery of cells and solid supports (e.g., beads) to the microwell array, a retractable platen or deformable membrane that is pressed into contact with the surface of the microwell array substrate during lysis or incubation steps, the use of larger beads, e.g. Sephadex beads as described previously, to block the openings of the microwells, or the release of an immiscible, hydrophobic fluid from a reservoir within the cartridge during lysis or incubation steps, to effectively separate and compartmentalize each microwell in the array.

The dimensions of fluid channels and the array chamber(s) in cartridge designs can be optimized to (i) provide uniform delivery of cells and beads to the microwell array, and (ii) to minimize sample and reagent consumption. The width of fluid channels can be between 50 micrometers and 20 mm. In other embodiments, the width of fluid channels can be at least 50 micrometers, at least 100 micrometers, at least 200 micrometers, at least 300 micrometers, at least 400 micrometers, at least 500 micrometers, at least 750 micrometers, at least 1 mm, at least 2.5 mm, at least 5 mm, at least 10 mm, or at least 20 mm. In yet other embodiments, the width of fluid channels can at most 20 mm, at most 10 mm, at most 5 mm, at most 2.5 mm, at most 1 mm, at most 750 micrometers, at most 500 micrometers, at most 400 micrometers, at most 300 micrometers, at most 200 micrometers, at most 100 micrometers, or at most 50 micrometers. The width of fluid channels can be about 2 mm. The width of the fluid channels can fall within any range bounded by any of these values (e.g. from about 250 μm to about 3 mm).

The fluid channels in the cartridge can have a depth. The depth of the fluid channels in cartridge designs can be between 50 micrometers and 2 mm. The depth of fluid channels can be at least 50 micrometers, at least 100 micrometers, at least 200 micrometers, at least 300 micrometers, at least 400 micrometers, at least 500 micrometers, at least 750 micrometers, at least 1 mm, at least 1.25 mm, at least 1.5 mm, at least 1.75 mm, or at least 2 mm. The depth of fluid channels can at most 2 mm, at most 1.75 mm, at most 1.5 mm, at most 1.25 mm, at most 1 mm, at most 750 micrometers, at most 500 micrometers, at most 400 micrometers, at most 300 micrometers, at most 200 micrometers, at most 100 micrometers, or at most 50 micrometers. The depth of the fluid channels can be about 1 mm. The depth of the fluid channels can fall within any range bounded by any of these values (e.g. from about 800 micrometers to about 1 mm).

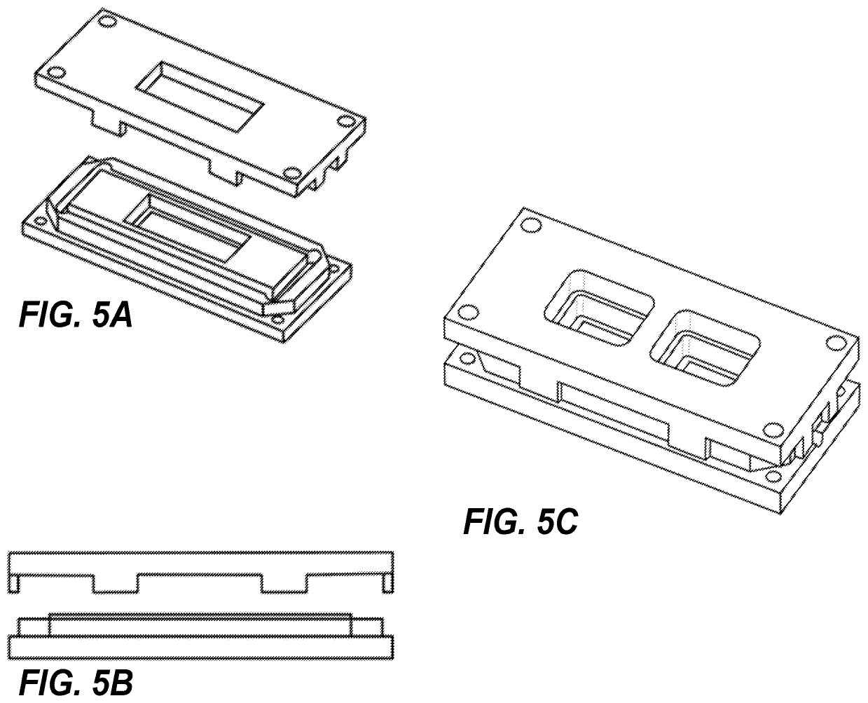

Cartridges can be fabricated using a variety of techniques and materials known to those of skill in the art. In general, the cartridges will be fabricated as a series of separate component parts (FIGS. 5A-5C) and subsequently assembled using any of a number of mechanical assemblies or bonding techniques. Examples of suitable fabrication techniques include, but are not limited to, conventional machining, CNC machining, injection molding, thermoforming, and 3D printing. Once the cartridge components have been fabricated they can be mechanically assembled using screws, clips, and the like, or permanently bonded using any of a variety of techniques (depending on the choice of materials used), for example, through the use of thermal bonding/welding or any of a variety of adhesives or adhesive films, including epoxy-based, acrylic-based, silicone-based, UV curable, polyurethane-based, or cyanoacrylate-based adhesives.

Cartridge components can be fabricated using any of a number of suitable materials, including but not limited to silicon, fused-silica, glass, any of a variety of polymers, e.g. polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COL), polyethylene terephthalate (PET), epoxy resins, non-stick materials such as teflon (PTFE), metals (e.g. aluminum, stainless steel, copper, nickel, chromium, and titanium), or any combination thereof.

The inlet and outlet features of the cartridge can be designed to provide convenient and leak-proof fluid connections with the instrument, or can serve as open reservoirs for manual pipetting of samples and reagents into or out of the cartridge. Examples of convenient mechanical designs for the inlet and outlet port connectors can include, but are not limited to, threaded connectors, Luer lock connectors, Luer slip or “slip tip” connectors, press fit connectors, and the like. The inlet and outlet ports of the cartridge can further comprise caps, spring-loaded covers or closures, or polymer membranes that can be opened or punctured when the cartridge is positioned in the instrument, and which serve to prevent contamination of internal cartridge surfaces during storage or which prevent fluids from spilling when the cartridge is removed from the instrument. The one or more outlet ports of the cartridge can further comprise a removable sample collection chamber that is suitable for interfacing with stand-alone PCR thermal cyclers or sequencing instruments.

The cartridge can include integrated miniature pumps or other fluid actuation mechanisms for control of fluid flow through the device. Examples of suitable miniature pumps or fluid actuation mechanisms can include, but are not limited to, electromechanically- or pneumatically-actuated miniature syringe or plunger mechanisms, membrane diaphragm pumps actuated pneumatically or by an external piston, pneumatically-actuated reagent pouches or bladders, or electro-osmotic pumps.

The cartridge can include miniature valves for compartmentalizing pre-loaded reagents or controlling fluid flow through the device. Examples of suitable miniature valves can include, but are not limited to, one-shot “valves” fabricated using wax or polymer plugs that can be melted or dissolved, or polymer membranes that can be punctured; pinch valves constructed using a deformable membrane and pneumatic, magnetic, electromagnetic, or electromechanical (solenoid) actuation, one-way valves constructed using deformable membrane flaps, and miniature gate valves.

The cartridge can include vents for providing an escape path for trapped air. Vents can be constructed according to a variety of techniques, for example, using a porous plug of polydimethylsiloxane (PDMS) or other hydrophobic material that allows for capillary wicking of air but blocks penetration by water.

The mechanical interface features of the cartridge can provide for easily removable but highly precise and repeatable positioning of the cartridge relative to the instrument system. Suitable mechanical interface features can include, but are not limited to, alignment pins, alignment guides, mechanical stops, and the like. The mechanical design features can include relief features for bringing external apparatus, e.g. magnets or optical components, into close proximity with the microwell array chamber (FIG. 5B).

The cartridge can also include temperature control components or thermal interface features for mating to external temperature control modules. Examples of suitable temperature control elements can include, but are not limited to, resistive heating elements, miniature infrared-emitting light sources, Peltier heating or cooling devices, heat sinks, thermistors, thermocouples, and the like. Thermal interface features can be fabricated from materials that are good thermal conductors (e.g. copper, gold, silver, etc.) and can comprise one or more flat surfaces capable of making good thermal contact with external heating blocks or cooling blocks.

The cartridge can include optical interface features for use in optical imaging or spectroscopic interrogation of the microwell array. The cartridge can include an optically transparent window, e.g. the microwell substrate itself or the side of the flow cell or microarray chamber that is opposite the microwell array, fabricated from a material that meets the spectral requirements for the imaging or spectroscopic technique used to probe the microwell array. Examples of suitable optical window materials can include, but are not limited to, glass, fused-silica, polymethylmethacrylate (PMMA), polycarbonate (PC), cyclic olefin polymers (COP), or cyclic olefin copolymers (COL).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

### EXAMPLES

Some aspects of the embodiments discussed above are disclosed in further detail in the following examples, which are not in any way intended to limit the scope of the present disclosure.

### Example 1

### Removing Amplification Noises

This example demonstrates removing amplification noises. After determining the amplification noises, the amplification noises were subtracted from the pre-correction numbers of targets in samples to generate the estimated numbers of targets.

### Experimental Protocol

Single cells were deposited into BD Precise™ 96-well plates that did not include defined barcoded primers (DBPs). Each well contained stochastic barcodes with different molecular labels and the same sample label to identify the sample origins. The stochastic barcodes were used as reverse transcription (RT) primers for generating complementary deoxyribonucleic acid (cDNA). The reaction volume per well during reverse transcription was 10 μL. The concentration of the defined barcoded primers during reverse transcription was 0.2 μM.

After reverse transcription, cDNAs from each well were pooled together into a single tube. Only about 8 μL of reaction mixture was recovered from the 10 μL reaction volume. After pooling, 8 μL of a defined barcoded primer mixture, including 4 DBPs at 0.2 uM each, was added into the pooled tube to create a combined reaction mixture. Primers were removed from the combined reaction mixture using Ampure XP beads (Beckman Coulter Inc. (Brea, CA)) for subsequent library preparation steps.

### Computation Error Correction

Table 1 shows experimental data of the sequencing readouts derived from 3 different types of defined barcoded that were mixed together: 1) oligo(dT) primers; 2) TRAC primers for T Cell Receptor Alpha C Region; and 3) TRBC primers for T Cell Receptor Beta C Region. Table 2 shows the 3 different types of defined barcoded primers, each with 4 different defined barcoded primers. The defined sample labels, bolded in Table 2, were used to identify the amplification noises from DBP crossover. The defined molecular labels are underlined.

The oligo(dT) primer captured poly(A) RNA transcripts, and TRAC and TRBC captured only the T Cell Receptor genes. For the oligo(dT)-primed cDNAs, approximately 100 genes were amplified into the final library. For each RT-primer derived read and each non-RT-primer derived read, the number of molecular labels within each read was determined. In Table 1, X01-X04 show the 4 defined barcoded primers used, and A01-A12 show to actual wells with samples.

TABLE 1
Sequencing readouts derived from 3 different RT primers

| | TRAC | TRBC | Oligo(dT) | TRAC | TRBC | Oligo(dT) |
|---|---|---|---|---|---|---|
| Sample | Reads | Reads | Reads | ML | MLs | MLs |
| X01 | 88 | 78 | 528 | 28 | 21 | 122 |
| X02 | 40 | 39 | 949 | 30 | 17 | 111 |
| X03 | 36 | 10 | 86 | 27 | 8 | 53 |
| X04 | 58 | 33 | 216 | 31 | 14 | 116 |
| A01 | 24957 | 7357 | 54806 | 214 | 87 | 827 |
| A02 | 656 | 171 | 20406 | 133 | 67 | 682 |
| A03 | 3136 | 3677 | 16791 | 104 | 52 | 445 |
| A04 | 16126 | 1332 | 11934 | 78 | 64 | 430 |
| A05 | 11847 | 15601 | 42007 | 115 | 128 | 589 |
| A06 | 7083 | 5172 | 39541 | 125 | 84 | 679 |
| A07 | 11646 | 83 | 30812 | 170 | 45 | 664 |
| A08 | 23720 | 13598 | 37545 | 175 | 100 | 666 |
| A09 | 4456 | 7463 | 13628 | 104 | 77 | 367 |
| A10 | 60 | 62 | 29830 | 37 | 36 | 426 |
| A11 | 12730 | 24718 | 28377 | 159 | 168 | 528 |
| A12 | 7472 | 2939 | 24293 | 111 | 56 | 537 |

TABLE 2
Sequences of defined barcoded primers mimicking Oligo(dT)
RT primers, TCR alpha RT primers, TCR beta RT primers

| | | Seq |
|---|---|---|
| | | ID |
| DBP Type | Defined Barcoded Primer Sequences | No |
| Mimicking | ACACGACGCTCTTCCGATCTAGCATCACNNNNNNNNTTTTTTTTTTTTT | 1 |
| Oligo(dT) | TTTTTV | |
| RT primers | | |
| | ACACGACGCTCTTCCGATCTCATATACANNNNNNNNTTTTTTTTTTTTT | 2 |
| | TTTTTV | |
| | ACACGACGCTCTTCCGATCTGTAATGTTNNNNNNNNTTTTTTTTTTTTT | 3 |
| | TTTTTV | |
| | ACACGACGCTCTTCCGATCTTGATTAGTNNNNNNNNTTTTTTTTTTTTT | 4 |
| | TTTTTV | |
| Mimicking | ACACGACGCTCTTCCGATCTAGCATCACNNNNNNNNCAGACAGACTTGT | 5 |
| TCR alpha | | |
| RT primers | | |
| | ACACGACGCTCTTCCGATCTCATATACANNNNNNNNCAGACAGACTTGT | 6 |
| | ACACGACGCTCTTCCGATCTGTAATGTTNNNNNNNNCAGACAGACTTGT | 7 |
| | ACACGACGCTCTTCCGATCTTGATTAGTNNNNNNNNCAGACAGACTTGT | 8 |
| Mimicking | ACACGACGCTCTTCCGATCTAGCATCACNNNNNNNNCCTTTTGGGTGTG | 9 |
| TCR beta | | |
| RT primers | | |
| | ACACGACGCTCTTCCGATCTCATATACANNNNNNNNCCTTTTGGGTGTG | 10 |
| | ACACGACGCTCTTCCGATCTGTAATGTTNNNNNNNNCCTTTTGGGTGTG | 11 |
| | ACACGACGCTCTTCCGATCTTGATTAGTNNNNNNNNCCTTTTGGGTGTG | 12 |

Once the amplification noise for each gene in the library was defined, a cross-panel noise deduction was performed to correct for PCR crossover errors according to Equation 1.

$$Final ML Read Count = ( ML Read Count from Signal wells ) - ( ML Read Count from DBP ) . \qquad ( 1 )$$

This subtraction was done on a gene-by-gene basis. For example, if Oligo (dT) RT primer was used to label GAPDH and CD3D and identified 40 and 8 MLs respectively, and the Oligo (dT) DBP identified 2 and 1 MI as noise, the final output would be 38 MI for GAPDH, and 7 MI for CD3D.

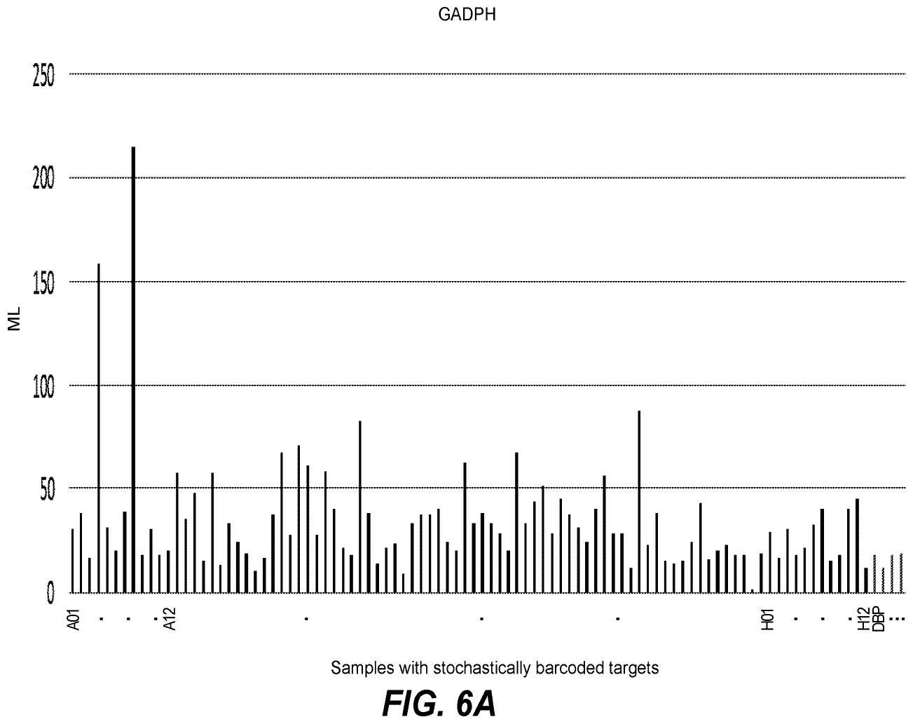

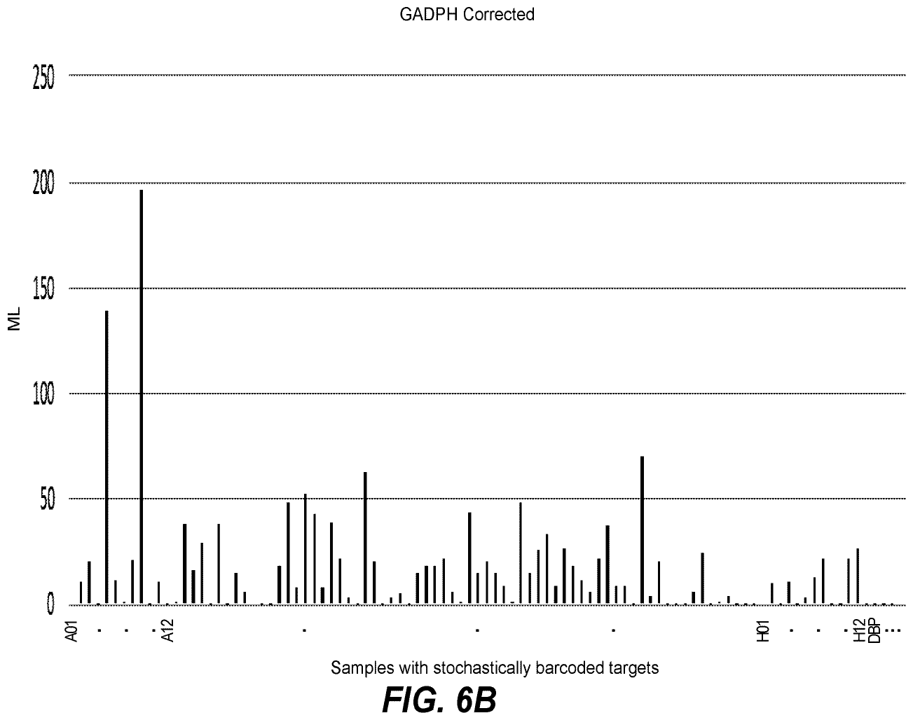

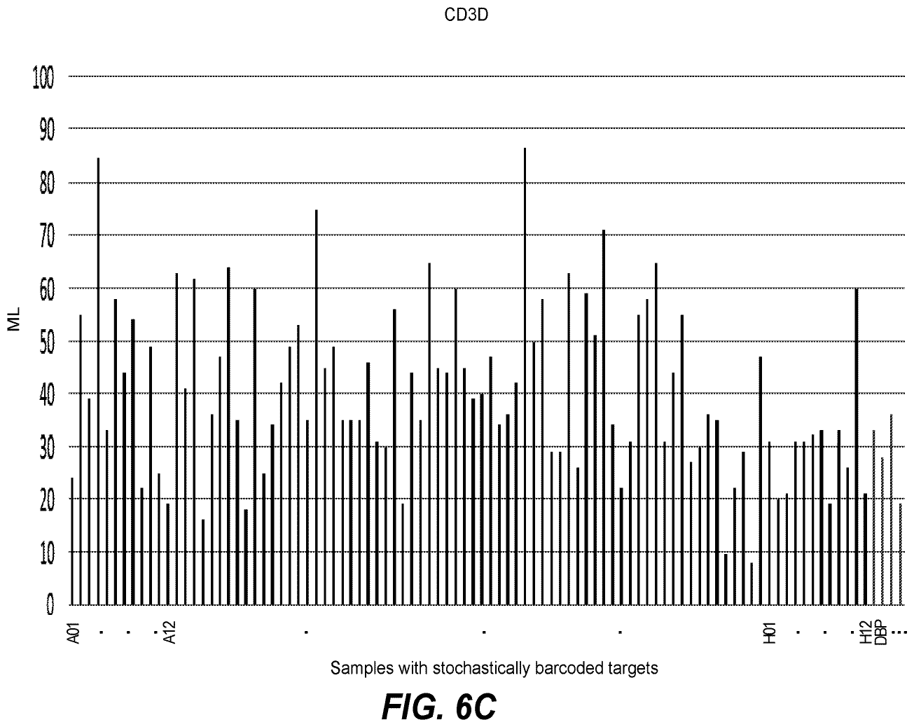

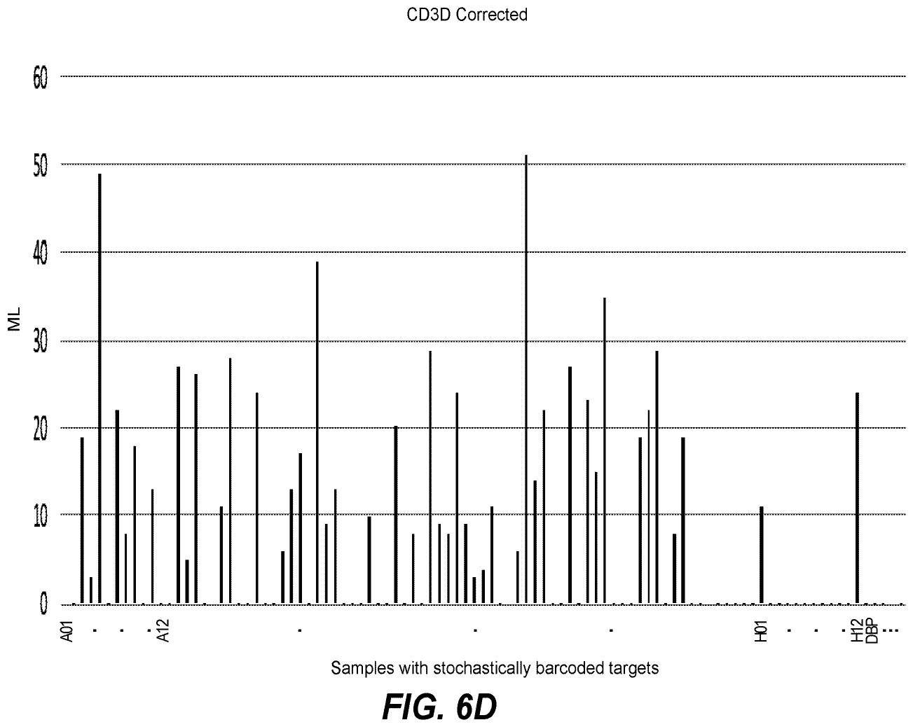

FIGS. 6A-D show bar charts of molecular label counts vs. wells for GAPDH and CD3D before and after correction for PCR amplification noises. FIGS. 6A-B show bar charts of molecular label counts vs. wells for GAPDH before (FIG. 6A) and after correction (FIG. 6B) for PCR amplification noises. FIGS. 6C-D show bar charts of molecular label counts vs. wells for CD3D before (FIG. 6C) and after correction (FIG. 6D) for PCR amplification noises.

Altogether these data demonstrate removing the amplification noises caused by PCR crossover to generate estimated numbers of targets.

### Example 2

### Defined Barcoded Primers Mimic No-Template Control Behavior

This example demonstrates that amplification noises during library preparation can be determined using defined barcoded primers.

The experimental protocol used in this example was similar to that used in Example 1. Briefly, single cells were deposited into some wells of BD Precise™ TCR 96-well plates with each well containing stochastic barcodes with different molecular labels and the same sample label to identify the sample origins. Wells without cells were no-template controls (NTCs). The stochastic barcodes were used as reverse transcription (RT) primers for generating complementary deoxyribonucleic acid (cDNA). After reverse transcription, cDNAs from each well were pooled together into a single tube. After pooling, DBPs shown in Table 2 were added into the pooled tube to create a combined reaction mixture. Primers were removed from the combined reaction mixture using Ampure XP beads (Beckman Coulter Inc. (Brea, CA)) for subsequent library preparation steps.

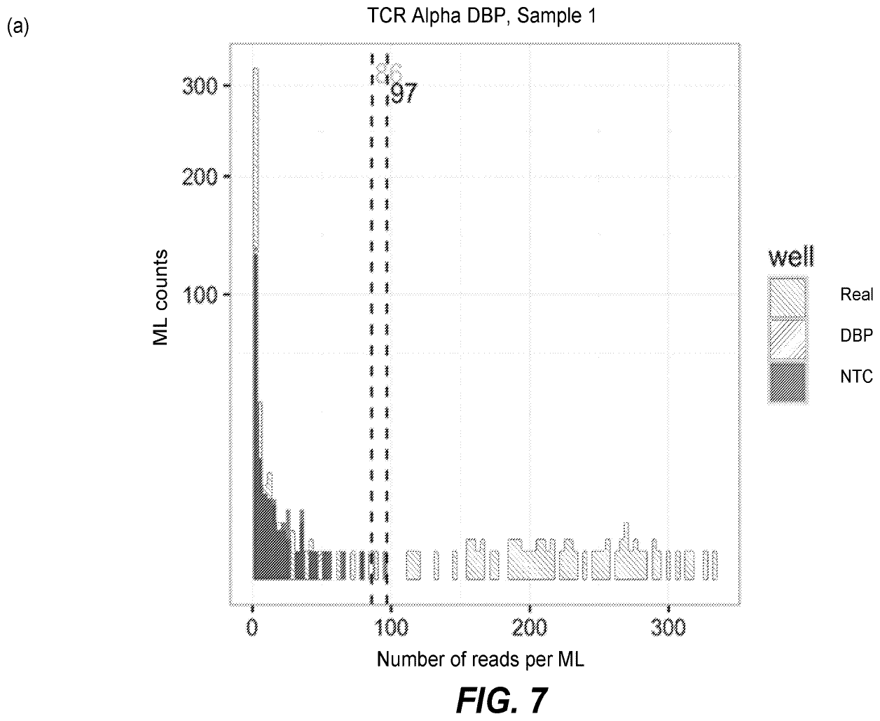

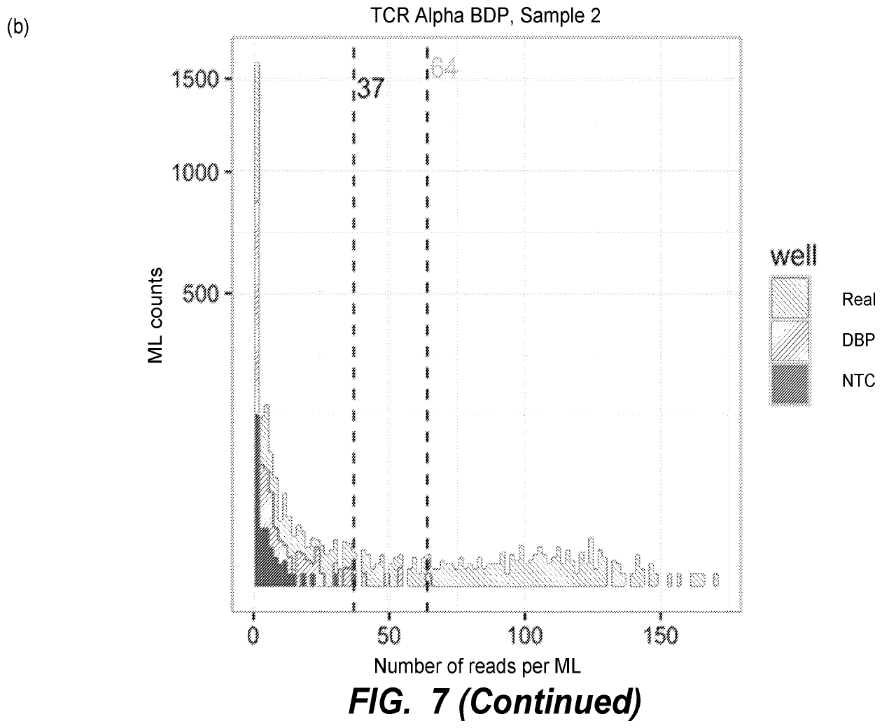

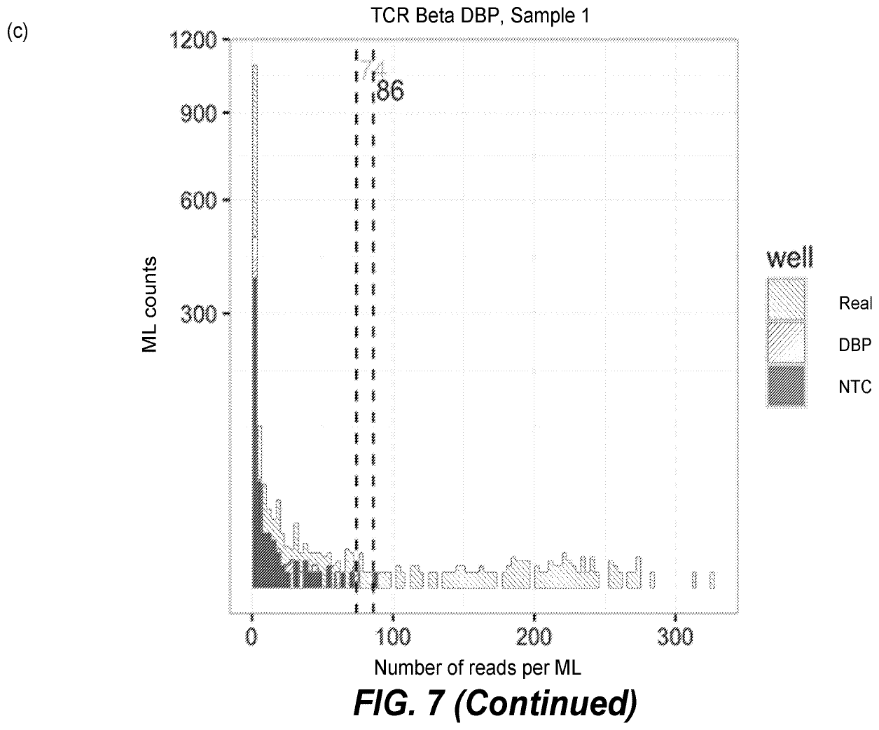

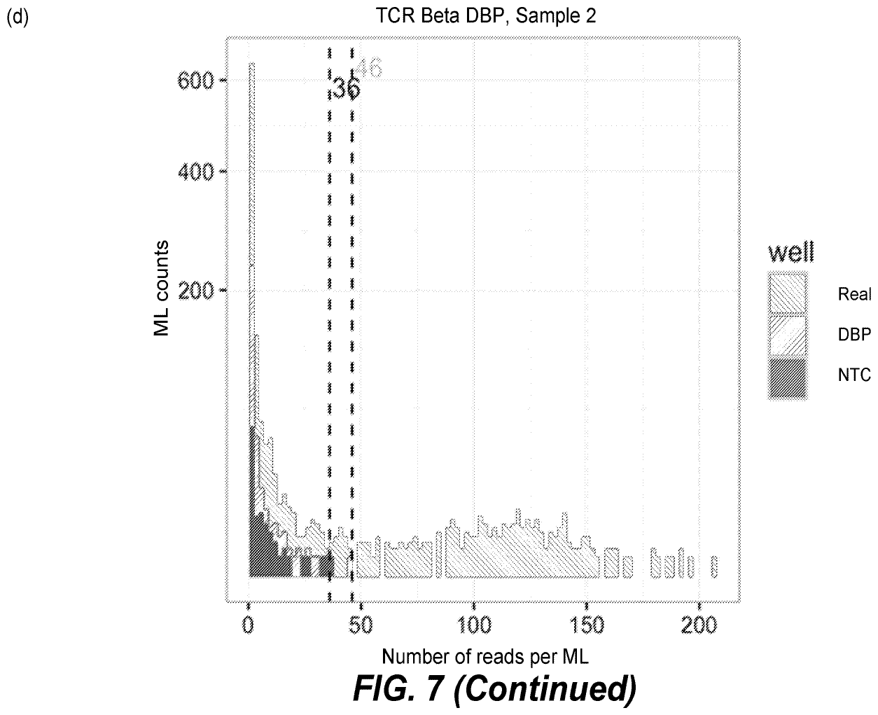

FIG. 7 contains non-limiting exemplary plots of molecular label counts vs. the number of reads per molecular label (ML), showing that the amplification noises determined using defined barcoded primers (DBPs) were similar to the noise levels determined using no-template controls (NTCs). FIG. 7 panels (a)-(b) show that the amplification noises determined using DBPs mimicking T-cell receptor (TCR) alpha (86 and 46 respectively for two samples) were similar to the noises determined using NTCs (97 and 36 respectively for the two samples). FIG. 7 panels (c)-(d) show that the amplification noises determined using DBPs mimicking TCR beta (74 and 64 respectively for two samples) were similar to the noises determined using NTCs (86 and 37 respectively for the two samples).

Altogether these data demonstrate that the amplification noises caused by during library preparation can be efficiently, easily, and accurately determined using defined barcoded primers. The amplification noises determined can in turn be used to filter numbers of targets to generate estimated numbers of targets.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for determining the number of targets in one or more samples, comprising:
stochastically barcoding a plurality of targets in each of one or more samples using a plurality of oligonucleotides comprising stochastic barcodes to generate stochastically barcoded targets, wherein for each sample, each stochastic barcode comprises a molecular label and an identical sample label, wherein the molecular labels of at least two stochastic barcodes differ from one another by at least one nucleotide;
contacting one or more defined barcoded primers with each of the one or more samples, wherein each of the one or more defined barcoded primers comprises a defined sample label and a defined molecular label, and wherein the defined sample labels are variants of the sample labels;
amplifying the stochastically barcoded targets and the one or more defined barcoded primers to generate a plurality of amplified stochastically barcoded targets and a plurality of amplified defined barcoded primers; and
estimating the number of each of the plurality of targets, wherein estimating the number of each of the plurality of targets comprises:
determining a pre-correction number of each of the plurality of targets using the molecular label;
determining an amplification noise by determining the number of defined molecular labels with different sequences in the plurality of amplified defined barcoded primers; and
removing the amplification noise from the pre-correction number of each of the plurality of targets to generate the estimated number of each of the plurality of targets.

2. The method of claim 1, wherein contacting the one or more defined barcoded primers with each of the one or more samples comprises introducing the one or more defined barcoded primers at the same concentration of the plurality of oligonucleotides comprising the stochastic barcodes.

3. The method of any one of claims 1-2, wherein the defined molecular label is 5-20 nucleotides in length.

4. The method of any one of claims 1-3, wherein the defined molecular label and the molecular label have the same length.

5. The method of any one of claims 1-4, wherein some of the molecular labels of the stochastic barcodes and some of the defined molecular labels of the one or more defined barcoded primers have the same sequence.

6. The method of any one of claims 1-5, wherein the defined sample label is 5-20 nucleotides in length.

7. The method of any one of claims 1-6, wherein the one or more defined barcoded primers comprise one or more types of defined barcoded primers.

8. The method of claim 7, wherein the defined molecular labels of one type of defined barcoded primers differ from one another by at least one nucleotide.

9. The method of any one of claims 7-8, wherein some defined molecular labels of different types of defined barcoded primers have the same sequence.

10. The method of any one of claims 7-9, wherein different types of defined barcoded primers have the same length.

11. The method of any one of claims 7-10, wherein the lengths of different types of defined barcoded primers differ by at most 10 nucleotides.

12. The method of any one of claims 7-11, wherein the defined sample labels of different types of defined barcoded primers have different sequences.

13. The method of any one of claims 1-12, wherein the sample label and the defined sample label have the same length.

14. The method of any one of claims 1-13, wherein the Hamming distance between the sample labels of the stochastic barcodes and the defined sample labels of the defined barcoded primers is at least 2.

15. The method of any one of claims 1-13, wherein the Hamming distance between the sample labels of the stochastic barcodes and the defined sample labels of the defined barcoded primers is at least 4.

16. The method of any one of claims 7-15, wherein the stochastic barcodes and the one or more types of defined barcoded primers have the same length.

17. The method of any one of claims 7-16, wherein the stochastic barcodes and the one or more types of defined barcoded primers have different lengths.

18. The method of any one of claims 1-17, further comprising removing at least 50% of the oligonucleotides comprising stochastic barcodes not incorporated into the stochastically barcoded targets and the one or more defined barcoded primers from each of the one or more samples.

19. The method of claim 18, wherein less than 10% of the unincorporated oligonucleotides comprising the stochastic barcodes are not removed from each of the one or more samples.

20. The method of any one of claims 18-19, wherein the percentage of the one or more defined barcoded primers not removed from each of the one or more samples is substantially the same as the percentage of the plurality of oligonucleotides comprising the stochastic barcodes not removed from each of the one or more samples.

21. The method of any one of claims 18-19, wherein the percentage of the one or more defined barcoded primers not removed from each of the one or more samples is within 10% of the percentage of the plurality of oligonucleotides comprising the stochastic barcodes not removed from each of the one or more samples.

22. The method of any one of claims 1-21, wherein amplifying the stochastically barcoded targets and the one or more defined barcoded primers to generate the plurality of amplified stochastically barcoded targets and the plurality of amplified defined barcoded primers comprises amplifying the stochastically barcoded targets and the one or more defined barcoded primers by polymerase chain reaction (PCR).

23. The method of any one of claims 1-22, wherein determining the pre-correction number of each of the plurality of targets using the molecular label comprises:
determining sequences of molecular labels of the amplified stochastically barcoded targets; and
counting the number of the molecular labels with different sequences.

24. The method of claim 23, wherein determining the sequences of the molecular labels of the amplified stochastically barcoded targets comprises sequencing some or all of the plurality of amplified stochastically barcoded targets.

25. The method of claim 24, wherein sequencing some or all of the amplified stochastically barcoded targets comprises generating sequences each with a read length of 100 or more bases.

26. The method of any one of claims 22-25, wherein determining the number of defined molecular labels with different sequences in the plurality of amplified defined barcoded primers comprises:
determining sequences of the defined molecular labels of the amplified defined barcoded primers; and
counting the number of defined molecular labels with different sequences.

27. The method of claim 26, wherein determining the sequences of the defined molecular labels of the amplified defined barcoded primers comprises sequencing some or all of the plurality of amplified defined barcoded primers.

28. The method of claim 27, wherein sequencing some or all of the plurality of amplified defined barcoded primers comprises generating sequences each with a read length of 100 or more bases.

29. The method of any one of claims 1-28, wherein determining the amplification noise comprises determining the number of defined molecular labels with different sequences in the plurality of amplified stochastically barcoded targets comprises determining the number of defined molecular labels in the plurality of amplified defined barcoded primers with different sequences for each type of amplified defined barcoded primers.

30. The method of any one of claims 1-28, wherein determining the amplification noise comprises determining the number of defined molecular labels with different sequences in the plurality of amplified stochastically barcoded comprises determining the average number of defined molecular labels in the plurality of amplified defined barcoded primers with different sequences for different types of amplified defined barcoded primers.

31. The method of any one of claims 1-28, wherein determining the amplification noise comprises determining the number of defined molecular labels with different sequences in the plurality of amplified stochastically barcoded targets and the plurality of amplified defined barcoded primers comprises determining the maximum number of defined molecular labels in the plurality of amplified stochastically barcoded targets and the plurality of amplified defined barcoded primers with different sequences for different types of amplified defined barcoded primers.

32. The method of any one of claims 1-31, wherein the amplification noise comprises noise caused by PCR crossover.

33. The method of any one of claims 1-32, further comprising pooling the stochastically barcoded targets to generate a pool of stochastically barcoded targets.

34. The method of any one of claims 1-33, wherein stochastically barcoding the plurality of targets comprises hybridizing the plurality of oligonucleotides comprising the stochastic barcodes with the plurality of targets to generate the stochastically barcoded targets.

35. The method of any one of claims 1-34, wherein the molecular label comprises 5-20 nucleotides.

36. The method of any one of claims 1-35, wherein the molecular labels of different stochastic barcodes are different from one another.

37. The method of any one of claims 1-36, wherein stochastically barcoding the plurality of targets is performed with a solid support comprising the plurality of oligonucleotides comprising the stochastic barcodes.

38. The method of claim 37, wherein the solid support comprises a plurality of synthetic particles associated with the plurality of oligonucleotides comprising the stochastic barcodes.

39. The method of claim 38, wherein the synthetic particles are beads.

40. The method of claim 39, wherein the beads are silica gel beads, controlled pore glass beads, magnetic beads, Dynabeads, Sephadex/Sepharose beads, cellulose beads, polystyrene beads, or any combination thereof.

41. The method of any one of claims 1-40, wherein the solid support comprises a polymer, a matrix, a hydrogel, a needle array device, an antibody, or any combination thereof.

42. The method of any one of claims 1-41, wherein the one or more samples comprise cells.

43. The method of claim 42, wherein at least one of the one or more samples comprises a single cell.

44. The method of any one of claims 42-43, further comprising lysing the cells.

45. The method of claim 44, wherein lysing the cells comprises heating the one or more samples, contacting the one or more samples with a detergent, changing the pH of the one or more samples, or any combination thereof.

46. The method of any one of claims 42-45, wherein the cells comprise one or more cell types.

47. The method of claim 46, wherein at least one of the one or more cell types is brain cell, heart cell, cancer cell, circulating tumor cell, organ cell, epithelial cell, metastatic cell, benign cell, primary cell, circulatory cell, or any combination thereof.

48. The method of any one of claims 1-47, wherein the plurality of targets comprise ribonucleic acids (RNAs), messenger RNAs (mRNAs), microRNAs, small interfering RNAs (siRNAs), RNA degradation products, RNAs each comprising a poly(A) tail, and any combination thereof.

49. The method of any one of claims 1-48, wherein the method is multiplexed.

50. A method for determining amplification noise, comprising:
reverse transcribing a plurality targets in each of one or more samples using oligonucleotides comprising reverse transcription primers to generate reverse transcribed targets;
contacting one or more defined barcoded primers with each of the one or more samples, wherein each of the one or more defined barcoded primers comprises a defined molecular label;
amplifying the reverse transcribed targets and the one or more defined barcoded primers to generate a plurality of amplified reverse transcribed targets and a plurality of amplified defined barcoded primers; and
determining an amplification noise by determining the number of defined molecular labels with different sequences in the plurality of defined barcoded primers.

51. The method of claim 50, wherein for each sample, each oligonucleotide comprises a stochastic barcode, wherein the stochastic barcode comprises a molecular label and an identical sample label, wherein the molecular labels of at least two plurality of stochastic barcodes differ from one another by at least one nucleotide,
wherein each of the one or more defined barcoded primers comprises a defined sample label, wherein the defined sample labels are variants of the sample label, and wherein the Hamming distance between the sample labels of stochastic barcodes and the defined sample labels of the defined barcoded primers is at least 2.

52. The method of any one of claims 50-51, wherein contacting the one or more defined barcoded primers with each of the one or more samples comprises introducing the one or more defined barcoded primers at the same concentration of the oligonucleotides comprising the reverse transcription primers.

53. The method of any one of claims 51-52, wherein the defined molecular label is 5-20 nucleotides in length.

54. The method of any one of claims 51-53, wherein the defined molecular label and the molecular label have the same length.

55. The method of any one of claims 51-54, wherein some of the molecular labels of the stochastic barcodes and some of the defined molecular labels of the one or more defined barcoded primers have the same sequence.

56. The method of any one of claims 51-55, wherein the defined sample label is 5-20 nucleotides in length.

57. The method of any one of claims 51-56, wherein the one or more defined barcoded primers comprise one or more types of defined barcoded primers.

58. The method of claim 57, wherein the defined molecular labels of one type of defined barcoded primers differ from one another by at least one nucleotide.

59. The method of any one of claims 57-58, wherein some defined molecular labels of different types of defined barcoded primers have the same sequence.

60. The method of any one of claims 57-59, wherein different types of defined barcoded primers have the same length.

61. The method of any one of claims 57-60, wherein the lengths of different types of defined barcoded primer differ by at most 10 nucleotides.

62. The method of any one of claims 57-61, wherein the sample labels of different types of defined barcoded primer have different sequences.

63. The method of any one of claims 51-62, wherein the sample label and the defined sample label have the same length.

64. The method of any one of claims 51-63, wherein the Hamming distance between the sample labels of the stochastic barcodes and the defined sample labels of the defined barcoded primers is at least 4.

65. The method of any one of claims 57-64, wherein the stochastic barcodes and the one or more types of defined barcoded primers have the same length.

66. The method of any one of claims 57-65, wherein the stochastic barcodes and the one or more types of defined barcoded primers have different lengths.

67. The method of any one of claims 50-66, further comprising removing at least 50% of the oligonucleotides comprising the reverse transcription primers not incorporated into the reverse transcribed targets and the one or more defined barcoded primers each of the one or more samples.

68. The method of claim 67, wherein less than 10% of the unincorporated oligonucleotides comprising the reverse transcription primers are not removed from each of the one or more samples.

69. The method of any one of claims 67-68, wherein the percentage of the one or more defined barcoded primers not removed from each of the one or more samples is substantially the same as the percentage of the oligonucleotides comprising the reverse transcription primers not removed from each of the one or more samples.

70. The method of any one of claims 67-68, wherein the percentage of the one or more defined barcoded primers not removed from each of the one or more samples is within 10% of the percentage of the oligonucleotides comprising the reverse transcription primers not removed from each of the one or more samples.

71. The method of any one of claims 50-70, wherein amplifying the reverse transcribed targets and the one or more defined barcoded primers to generate the plurality of amplified reverse transcribed targets and the plurality of amplified defined barcoded primers comprises amplifying the reverse transcribed targets and the one or more defined barcoded primers by polymerase chain reaction (PCR)

72. The method of any one of claims 50-71, wherein determining the number of defined molecular labels with different sequences in the plurality of amplified defined barcoded primers comprises:
determining sequences of defined molecular labels of the amplified defined barcoded primers; and
counting the number of defined molecular labels with different sequences for each type amplified defined barcoded primers.

73. The method of claim 72, wherein determining the sequences of the defined molecular labels of the amplified defined barcoded primers comprises sequencing some or all of the amplified defined barcoded primers in the plurality of amplified defined barcoded primers.

74. The method of claim 73, wherein sequencing some or all of the amplified defined barcoded primers comprises generating sequences each with a read length of 100 or more bases.

75. The method of any one of claims 50-74, wherein determining the number of defined molecular labels with different sequences in the plurality of amplified reverse transcribed targets comprises determining the number of defined molecular labels in the plurality of amplified reverse transcribed targets with different sequences for each type of amplified defined barcoded primers.

76. The method of any one of claims 50-74, wherein determining the number of defined molecular labels with different sequences in the plurality of amplified defined barcoded primers comprises determining the average number of the molecular labels with different sequences for different types of amplified defined barcoded primers.

77. The method of any one of claims 50-74, wherein determining the number of defined molecular labels with different sequences in the plurality of amplified defined barcoded primers comprises determining the maximum number of the molecular labels with different sequences for different types of amplified defined barcoded primers.

78. The method of any one of claims 50-77, wherein the amplification noise comprises noise caused by PCR crossover.

79. The method of any one of claims 50-78, further comprising pooling the reverse transcribed targets to generate a pool of reverse transcribed targets.

80. The method of any one of claims 50-79, further comprising estimating the number of each of the plurality of targets.

81. The method of claim 80, wherein estimating the number of each of the plurality of targets comprises:
determining sequences of molecular labels of the amplified reverse transcribed targets; and
counting the number of the molecular labels with different sequences.

82. The method of any one of claims 80-81, wherein estimating the number of each of the plurality of targets comprises:
determining a pre-correction number of each of the targets using the molecular label; and
removing the amplification noise from the pre-correction number of each of the plurality of targets to generate the estimated number of each of the targets.

83. The method of any one of claims 51-82, wherein reverse transcribing the plurality of targets using oligonucleotides comprising reverse transcription primers comprises hybridizing the oligonucleotides comprising the reverse transcription primers with the plurality of targets to generate the reverse transcribed targets.

84. The method of claim 83, wherein the molecular label comprises 5-20 nucleotides.

85. The method of any one of claims 83-84, wherein the molecular labels of different reverse stochastic barcodes are different from one another.

86. The method of any one of claims 50-85, wherein reverse transcribing the plurality of targets using the reverse transcription primers is performed with a solid support comprising the oligonucleotides comprising the reverse transcription primers.

87. The method of claim 86, wherein the solid support comprises a plurality of synthetic particles associated with the oligonucleotides comprising the reverse transcription primers.

88. The method of claim 87, wherein the synthetic particles are beads.

89. The method of claim 88, wherein the beads are silica gel beads, controlled pore glass beads, magnetic beads, Dynabeads, Sephadex/Sepharose beads, cellulose beads, polystyrene beads, or any combination thereof.

90. The method of any one of claims 86-89, wherein the solid support comprises a polymer, a matrix, a hydrogel, a needle array device, an antibody, or any combination thereof.

91. The method of any one of claims 50-90, wherein the one or more samples comprise cells.

92. The method of claim 91, wherein at least one of the one or more samples is a single cell.

93. The method of any one of claims 91-92, further comprising lysing the cells.

94. The method of claim 93, wherein lysing the cells comprises heating the one or more samples, contacting the one or more samples with a detergent, changing the pH of the one or more samples, or any combination thereof.

95. The method of any one of claims 91-94, wherein the cells comprise one or more cell types.

96. The method of claim 95, wherein at least one of the one or more cell types is brain cell, heart cell, cancer cell, circulating tumor cell, organ cell, epithelial cell, metastatic cell, benign cell, primary cell, circulatory cell, or any combination thereof.

97. The method of any one of claims 50-95, wherein the plurality of targets comprise ribonucleic acids (RNAs), messenger RNAs (mRNAs), microRNAs, small interfering RNAs (siRNAs), RNA degradation products, RNAs each comprising a poly(A) tail, and any combination thereof.

98. The method of any one of claims 50-97, wherein the method is multiplexed.

99. A method for determining the number of targets in microwells of a microwell array, comprising:
stochastically barcoding a plurality of targets in each of one or more samples in microwells of a microwell array using a plurality of oligonucleotides comprising stochastic barcodes to generate stochastically barcoded targets, wherein for each well, each stochastic barcode comprises a molecular label and an identical sample label, wherein the molecular labels of at least two stochastic barcodes differ from one another by at least one nucleotide;
pooling the stochastically barcoded targets from the microwells of the microwell array to generate a pool of stochastically barcoded targets;
contacting defined barcoded primers of one or more types of defined barcoded primers with each of the one or more samples, wherein each defined barcoded primer comprises a defined sample label and a defined molecular label, wherein the defined sample labels of defined barcoded primers of the same type of defined barcoded primers have the same sequence, wherein the defined sample labels of different types of defined barcoded primers have different sequences, wherein the sample labels of the stochastic barcodes and the defined sample labels are variants of the sample label, and wherein the Hamming distance between the sample labels of the stochastic barcodes and the defined sample labels of the defined barcoded primers is at least 2;
amplifying the stochastically barcoded targets and the one or more defined barcoded primers to generate a plurality of amplified stochastically barcoded targets and a plurality of amplified defined barcoded primers; and
estimating the number of each of the plurality of targets, wherein estimating the number of each of the plurality of targets comprises:
determining a pre-correction number of each of the plurality of targets using the molecular label;
determining an amplification noise by determining the number of defined molecular labels with different sequences in the plurality of amplified defined barcoded primers; and
removing the amplification noise from the pre-correction number of each of the plurality of targets to generate the estimated number of each of the plurality of targets.

100. The method of claim 99, wherein contacting the defined barcoded primers with each of the one or more samples comprises introducing the defined barcoded primers at the same concentration of the plurality of oligonucleotides comprising the stochastic barcodes.

101. The method of any one of claims 99-100, wherein the defined molecular label is 5-20 nucleotides in length.

102. The method of any one of claims 99-101, wherein the defined molecular label and the molecular label have the same length.

103. The method of any one of claims 99-102, wherein the molecular labels of some of the stochastic barcodes and the defined molecular labels of some of the defined barcoded primers have the same sequence.

104. The method of any one of claims 99-103, wherein the defined sample label is 5-20 nucleotides in length.

105. The method of any one of claims 99-104, wherein the defined molecular labels of some defined barcoded primers of different types of defined barcoded primers have the same sequence.

106. The method of any one of claims 99-105, wherein the defined barcoded primers of different types of defined barcoded primers have the same length.

107. The method of any one of claims 99-105, wherein the lengths of different types of defined barcoded primers differ by at most 10 nucleotides.

108. The method of any one of claims 99-107, wherein the sample label and the defined sample label have the same length.

109. The method of any one of claims 99-108, the Hamming distance between the sample labels of the stochastic barcodes and the defined sample labels of the defined barcoded primers is at least 4.

110. The method of any one of claims 99-109, wherein the stochastic barcodes and the defined barcoded primers have the same length.

111. The method of any one of claims 99-109, wherein the stochastic barcodes and some of the defined barcoded primers have different lengths.

112. The method of any one of claims 99-111, further comprising removing at least 50% of the oligonucleotides comprising stochastic barcodes not incorporated into the stochastically barcoded targets and the one or more types of defined barcoded primers from each of the one or more samples.

113. The method of claim 112, wherein less than 10% of the unincorporated oligonucleotides comprising the stochastic barcodes are not removed from each of the one or more samples.

114. The method of any one of claims 112-113, wherein the percentage of the one or more defined barcoded primers not removed from each of the one or more samples is substantially the same as the percentage of the plurality of oligonucleotides comprising the stochastic barcodes not removed from each of the one or more samples.

115. The method of any one of claims 112-113, wherein the percentage of the one or more defined barcoded primers not removed from each of the one or more samples is within 10% of the percentage of the plurality of oligonucleotides comprising the stochastic barcodes not removed from each of the one or more samples.

116. The method of any one of claims 99-115, wherein amplifying the stochastically barcoded targets and the one or more types of defined barcoded primers to generate the plurality of amplified stochastically barcoded targets and the one or more defined barcoded primers comprises amplifying the stochastically barcoded targets and the one or more types defined barcoded primers by polymerase chain reaction (PCR).

117. The method of any one of claims 99-116, wherein determining the pre-correction number of each of the plurality of targets using the molecular label comprises:
determining sequences of molecular labels of the amplified stochastically barcoded targets; and
counting the number of the molecular labels with different sequences.

118. The method of claim 117, wherein determining the sequences of the molecular labels of the amplified stochastically barcoded targets comprises sequencing some or all of the plurality of amplified stochastically barcoded targets.

119. The method of claim 118, wherein sequencing some or all of the amplified stochastically barcoded targets comprises generating sequences each with a read length of 100 or more base.

120. The method of any one of claims 99-119, wherein determining the number of defined molecular labels with different sequences in the plurality of amplified defined barcoded primers comprises:
determining sequences of the defined molecular labels of the amplified defined barcoded primers; and
counting the number of defined molecular labels with different sequences.

121. The method of claim 120, wherein determining the sequences of the defined molecular labels of the amplified defined barcoded primers comprises sequencing some or all of the plurality of amplified defined barcoded primers.

122. The method of claim 121, wherein sequencing some or all of the plurality of amplified defined barcoded primers comprises generating sequences each with a read length of 100 or more bases.

123. The method of any one of claims 99-122, wherein estimating the amplification noise comprises determining the number of defined molecular labels with different sequences in the plurality of amplified defined barcoded primers comprises determining the number of defined molecular labels in the plurality of amplified defined barcoded primers with different sequences for each type of amplified defined barcoded primers.

124. The method of any one of claims 99-122, wherein estimating the amplification noise comprises determining the number of defined molecular labels with different sequences in the amplified defined barcoded primers comprises determining the average number of defined molecular labels in the plurality of amplified defined barcoded primers with different sequences for different types of amplified defined barcoded primers.

125. The method of any one of claims 99-122, wherein estimating the amplification noise comprises determining the number of defined molecular labels with different sequences in the plurality of amplified defined barcoded primers comprises determining the maximum number of defined molecular labels in the plurality of amplified defined barcoded primers with different sequences for different types of amplified defined barcoded primers.

126. The method of any one of claims 99-125, wherein the amplification noise comprises noise caused by PCR crossover.

127. The method of any one of claims 99-126, further comprising pooling the stochastically barcoded targets to generate a pool of stochastically barcoded targets.

128. The method of any one of claims 99-127, wherein stochastically barcoding the plurality of targets comprises hybridizing the plurality of oligonucleotides comprising the stochastic barcodes with the plurality of targets to generate the stochastically barcoded targets.

129. The method of any one of claims 99-128, wherein the molecular label comprises 5-20 nucleotides.

130. The method of any one of claims 99-129, wherein stochastically barcoding the plurality of targets in each of one or more samples in the microwells of the microwell array is performed with a solid support comprising the plurality of oligonucleotides comprising the stochastic barcodes.

131. The method of claim 130, wherein the solid support comprises a plurality of synthetic particles associated with the plurality of stochastic barcodes.

132. The method of claim 131, wherein the synthetic particles are beads.

133. The method of claim 132, wherein the beads are silica gel beads, controlled pore glass beads, magnetic beads, Dynabeads, Sephadex/Sepharose beads, cellulose beads, polystyrene beads, or any combination thereof.

134. The method of any one of claims 99-133, wherein the solid support comprises a polymer, a matrix, a hydrogel, a needle array device, an antibody, or any combination thereof.

135. The method of any one of claims 99-134, wherein the one or more samples comprise cells.

136. The method of claim 135, wherein at least one of the one or more samples is a single cell

137. The method of any one of claims 135-136, further comprising lysing the cells.

138. The method of claim 137, wherein lysing the cells comprises heating the one or more samples, contacting the one or more samples with a detergent, changing the pH of the one or more samples, or any combination thereof.

139. The method of any one of claims 135-138, wherein the cells comprise one or more cell types.

140. The method of claim 139, wherein at least one of the one or more cell types is brain cell, heart cell, cancer cell, circulating tumor cell, organ cell, epithelial cell, metastatic cell, benign cell, primary cell, circulatory cell, or any combination thereof.

141. The method of any one of claims 99-140, wherein the plurality of targets comprise ribonucleic acids (RNAs), messenger RNAs (mRNAs), microRNAs, small interfering RNAs (siRNAs), RNA degradation products, RNAs each comprising a poly(A) tail, and any combination thereof.

142. The method of any one of claims 99-141, wherein the method is multiplexed.

143. A kit comprising:
a plurality of oligonucleotides comprising stochastic barcodes, wherein each stochastic barcode comprises a sample label and a molecular label, wherein the sample labels of at least two stochastic barcodes have different sequences, and wherein the molecular labels of stochastic barcodes with sample labels of the same sequence differ from one another by at least one nucleotide;
defined barcoded primers, wherein each defined barcoded primer comprises a defined sample label and a defined molecular label, wherein the defined sample labels of defined barcoded primers are variants of the sample labels of the stochastic barcodes, and wherein the Hamming distance between the sample label and the defined sample label is at least 2; and
instructions for using the plurality oligonucleotides comprising the plurality of oligonucleotides and the defined barcoded primers.

144. The kit of claim 143, wherein the defined sample label is 5-20 nucleotides in length and the defined molecular label is 5-20 nucleotides in length.

145. The kit of any one of claim 143, wherein the defined molecular label and the molecular label have the same length.

146. The kit of any one of claims 143-145, wherein different types of defined barcoded primers have the same length.

147. The kit of any one of claims 143-146, wherein the lengths of different types of defined barcoded primers differ by at most 10 nucleotides.

148. The kit of any one of claims 143-147, wherein the sample label and the defined sample label have the same length.

149. The kit of any one of claims 143-148, wherein the Hamming distance between the sample label and the defined sample label is at least 2.

150. The kit of any one of claims 143-148, wherein the Hamming distance between the sample label and the defined sample label is at least 4.

151. The kit of any one of claims 143-150, wherein the stochastic barcodes and the defined barcoded primers have the same length.

152. The kit of any one of claims 143-150, wherein the stochastic barcodes and the defined barcoded primers have different lengths.

153. The kit of any one of claims 143-152, wherein the plurality of oligonucleotides comprising the stochastic barcodes is associated with a solid support.

154. The kit of claim 153, wherein the solid support comprises a plurality of synthetic particles associated with the plurality of oligonucleotides comprising stochastic barcodes.

155. The kit of claim 154, wherein the plurality of synthetic particles is beads.

156. The kit of claim 155, wherein the beads are silica gel beads, controlled pore glass beads, magnetic beads, Dynabeads, Sephadex/Sepharose beads, cellulose beads, polystyrene beads, or any combination thereof.

157. The kit of any one of claims 154-156, wherein the synthetic particles are magnetic beads.

158. The kit of any one of claims 153-157, wherein the solid support comprises a polymer, a matrix, a hydrogel, a needle array device, an antibody, or any combination thereof.

159. The kit of any one of claims 143-158, further comprising a buffer.

160. The kit of any one of claims 143-159, further comprising a cartridge.

161. The kit of any one of claims 153-160, wherein the solid support is pre-loaded on a substrate.

162. The kit of any one of claims 143-161, further comprising one or more reagents for a reverse transcription reaction.

163. The kit of any one of claims 143-162, further comprising one or more reagents for an amplification reaction.
